(19) United States
(12) Patent Application Publication
(10) Pub. No.: US 2012/0329049 A1
(43) Pub. Date: Dec. 27, 2012

(54) BCR-ABL1 SPLICE VARIANTS AND USES THEREOF

(76) Inventors: Wanlong Ma, Aliso Viejo, CA (US); Maher Albitar, Coto De Caza, CA (US)

(21) Appl. No.: 13/512,945

(22) PCT Filed: Dec. 3, 2010
(86) PCT No.: PCT/US10/58987

§ 371 Date: Jun. 28, 2012

Related U.S. Application Data
(60) Provisional application No. 61/266,872, filed on Dec. 4, 2009.

(51) Int. Cl. G01N 33/574; C12N 15/79; C12N 15/86; C12Q 1/68; C12N 15/63

(52) U.S. Cl. 435/6.11; 435/320.1; 435/7.92; 435/7.1; 436/501

(57) ABSTRACT

The present invention is based on BCR-ABL1 splice variants which result from insertion and/or truncation of the BCR-ABL1 transcript and the finding that these variants provide resistance to kinase domain inhibitors such as imatinib, nilotinib and dasatinib.

### CROSS-REFERENCE TO RELATED APPLICATIONS

This application claims benefit of U.S. Provisional 61/266,872, filed Dec. 4, 2009, hereby incorporated by reference in its entirety.

### FIELD OF THE INVENTION

The present invention relates to BCR-ABL1 variants and resistance to kinase inhibitor therapy.

### BACKGROUND OF THE INVENTION

The following description is provided to assist the understanding of the reader. None of the information provided or references cited is admitted to be prior art to the present invention.

Chronic Myelogenous Leukemia (“CML”) is a cancer of bone marrow and blood cells. In CML, healthy bone marrow cells are replaced with leukemic cells; myeloid, erythroid, megakaroyocytic and B lymphoid cells are among the blood cells which become leukemic due to the effects of a characteristic chromosomal translocation.

CML is associated with a specific chromosomal abnormality called Philadelphia chromosome. The genetic defect is caused by the reciprocal translocation designated t(9; 22)(q34; q11), which refers to an exchange of genetic material between region q34 of chromosome 9 and region q11 of chromosome 22 (Rowley, J. D. Nature. 1973; 243: 290-3; Kurzrock et al. N. Engl. J. Med. 1988; 319: 990-998). This translocation results in a portion of the bcr (“breakpoint cluster region”) gene from chromosome 22 (region q11) becoming fused with a portion of the abl1 gene on chromosome 9 (region q34). (Wong & Witte, Annu. Rev. Immunol. 2004; 22: 247-306).

The fused “bcr-ab1” gene is located on chromosome 22, which is shortened as a result of the translocation. The fused gene retains the tyrosine kinase domain of the abl gene, which is constitutively active (Elefanty et al. EMBO J. 1990; 9: 1069-1078). This kinase activity activates various signal transduction pathways leading to uncontrolled cell growth and division (e.g., by promoting cell proliferation and inhibiting apoptosis). For example, BCR-ABL may cause undifferentiated blood cells to proliferate and fail to mature.

Alternative BCR/ABL splice variants in Philadelphia chromosome-positive CML patient have been reported. Specifically, alternative splice variants between BCR exon 1, 13 and ABL exon 4 or 5 were reported by Volpe et al., (Cancer Res. 67:5300-07 (2007).

Treatment of CML may involve drug therapy (e.g., chemotherapy), bone marrow transplants, or combinations of both. One class of drugs that may be used for treating CML is kinase inhibitors. For example, “imatinib mesylate” (also known as STI571 or 2-phenylaminopyrimidine or “Imatinib”) has proven effective for treating CML (Deininger et al., Blood. 1997; 90: 3691-3698; Manley, P. W., Eur. J. Cancer. 2002; 38: S19-S27). Imatinib is marketed as a drug under the trade name “Gleevec” or “Glivec.” Other examples of kinase inhibitor drugs for treating CML include nilotinib, dasatinib, Bosutinib (SKI-606) and Aurora kinase inhibitor VX-680.

Imatinib is an ATP competitive inhibitor of BCR-ABL1 kinase activity and functions by binding to the kinase domain of BCR-ABL1 and stabilizing the protein in its closed, inactive conformation. Monotherapy with imatinib has been shown to be effective for all stages of CML.

Resistance to imatinib, and other kinase inhibitors, remains a major problem in the management of patients with CML. Rates at which primary (failure to achieve any hematologic response) and secondary resistance (i.e., hematologic recurrence) occurs varies dependent on the stage of diseases. Primary resistance has been reported in chronic-, accelerated-, or blast-phase at rates of 3%, 9%, and 51%, respectively (Melo, J. V. & Chuah, C. Cancer Lett. 2007; 249: 121-132; Hughes, T. Blood. 2006; 108: 28-37). Secondary resistance has been reported in these patients at rates of 22%, 32%, and 41%, respectively.

The complete mechanism of kinase inhibitor resistance in CML patients is unclear and a significant number of patients resistant to imatinib have no mutation in the bcr-abl1 gene. However, 35-45% of patients with imatinib resistance have mutations in the kinase domain of the BCR-ABL1 protein (Mahon, F. X. Blood. 2000; 96: 1070-1079). Most of the reported mutations disrupt critical contact points between imatinib and the tyrosine kinase receptor or induce a transition from the inactive to the active protein configuration, preventing imatinib binding (Nagar, B. Cell. 2003; 112: 859-871; Nagar et al., Cancer Res. 2002; 62: 4236-4243; Branford S. Blood. 2002; 99: 3472-3475; Branford et al. Blood. 2003; 102: 276-283; O'Hare et al., Blood 2007 110: 2242-2249 (2007)).

The T315I mutation (Gone et al. Science. 2001; 293: 876-880; Hochhaus et al. Leukemia. 2002; 16: 2190-2196) and some mutations affecting the P-loop of BCR-ABL1 confer a greater level of resistance to imatinib (Branford et al. Blood. 2002; 99: 3472-3475; Branford et al. Blood. 2003; 102: 276-283; and Gone et al. Blood. 2002; 100: 3041-3044) as well as other tyrosine kinase inhibitors that are currently used and tested in these patients (Hughes et al. Blood. 2006; 108: 28-37; Hochhaus, et al. Blood. 2006; 108: 225a). The role of Src family kinases has received particular interest as possible mechanism for imatinib resistance (Levinson et al. PLoS Biol. 2006; 4: e144). Overexpression and activation of the Lyn has been implicated in imatinib-resistance (Donato, N. J. Blood. 2003; 101: 690-698).

Furthermore, Chu et al. (N. Engl. J. Med. 2006; 355: 10) describe an insertion/truncation mutant of BCR-ABL1 in a CML patient resistant to imatinib. Chu et al. report that the mutant results from a 35 base insertion of abl1 intron 8 into the junction between exons 8 and 9, resulting in a new C-terminus and truncation of the normal C-terminus of the ABL1 portion of the fusion protein. Laudadio et al. (J. Mol. Diag. 2008; 10(2): 177-180) and Lee et al. (Mol. Cancer. Ther. 2008; 7(12): 3834-41) also report a similar splice variant in CML patients that had undergone imatinib therapy. An additional splice variant without c-ABL exon 7 has also been reported in Imatinib-resistant patients. Curvo et al., Leuk. Res. 2007; 32:508-510.

### SUMMARY OF THE INVENTION

The present inventions are based on BCR-ABL1 splice variants which result from insertion and/or truncation of the bcr-abl transcript and the finding that these variants provide resistance to kinase domain inhibitors such as imatinib, nilotinib and dasatinib.

In one aspect, the invention provides a method for predicting likelihood for resistance of a patient with a Bcr/Abl translocation to treatment with one or more BCR/Abl kinase inhibitors, comprising: (a) assessing the bcr-abl mRNA in a sample obtained from the patient for the presence or absence of a polynucleotide sequence encoding the 79INS BCR/Abl splice variant, the 84INS BCR/Abl splice variant, or the 231 BCR/Abl splice variant; and (b) identifying the patient as having an increased likelihood of being resistant to treatment with one or more BCR/ABL kinase inhibitors when a polynucleotide encoding at least one of said splice variants is detected.

In some embodiments, the presence or absence of the 79INS BCR/Abl splice variant is determined by detecting the presence or absence of a BCR/Abl nucleic acid comprising the sequence of SEQ ID NO: 9.

In another aspect, the invention provides a method for predicting likelihood for resistance of a patient with a Bcr/Abl translocation to treatment with one or more BCR-Abl kinase inhibitors, comprising: (a) assessing the BCR-Abl protein in a sample obtained from a patient for the presence or absence of a truncated Abl protein encoded the 84INS BCR/Abl splice variant or the 231INS BCR/Abl splice variant; and (b) identifying the patient as having an increased likelihood of being resistant to treatment with one or more BCR-ABL kinase inhibitors when at least one of said truncated proteins is detected.

In some embodiments, the presence or absence of the BCR/Abl protein encoded by the BCR/Abl splice variants is determined by detecting the size of the BCR/Abl protein(s) in the patient sample, or by using an antibody that specifically binds to the BCR-ABL protein encoded by the 84INS BCR/Abl splice variant or the 231INS BCR/Abl splice variant, or by sequencing the C-terminus of the BCR-Abl protein. In some embodiments, the C-terminus of the Abl protein encoded the 84INS BCR/Abl splice variant comprises the amino acid sequence of SEQ ID NO: 4 and/or the C-terminus of the Abl protein encoded the 231INS BCR/Abl splice variant comprises the amino acid sequence of SEQ ID NO: 5.

In some embodiments of either of the foregoing aspects, the patient is diagnosed as having a myeloproliferative disease (e.g., chronic myelogenous leukemia (CML)). In other embodiments, the kinase inhibitors include one or more of imatinib, nilotinib, bosutinib, and dasatinib. In other embodiments, the sample is a blood or bone marrow. Preferably, the sample contains blood cells (e.g., peripheral mononuclear cells) and/or platelets.

In another aspect, the invention provides a recombinant polynucleotide which encodes the 79INS BCR-Abl splice variant, the 84INS splice variant, or the 231 BCR-Abl splice variant. In some embodiment, the recombinant polynucleotide is operably linked to an expression regulatory element capable of modulating the expression of said recombinant polynucleotide. The regulatory element optionally contains a promoter, an enhancer, and/or a poly-adenylation signal. The vectors include expression vectors and may be eukaryotic, prokaryotic, or viral.

The term “myeloproliferative disease” as used herein means a disorder of a bone marrow-derived cell type, such as a white blood cell. A myeloproliferative disease is generally manifest by abnormal cell division resulting in an abnormal level of a particular hematological cell population. The abnormal cell division underlying a myeloproliferative disease is typically inherent in the cells and not a normal physiological response to infection or inflammation. A leukemia is a type of myeloproliferative disease. Exemplary myeloproliferative disease include, but are not limited to, acute myeloid leukemia, acute lymphoblastic leukemia, chronic lymphocytic leukemia, myelodysplastic syndrome, chronic myeloid leukemia, hairy cell leukemia, leukemic manifestations of lymphomas, and multiple myeloma.

As used herein, the term “sample” or “biological sample” refers to any liquid or solid material obtained from a biological source, such a cell or tissue sample or bodily fluids. “Bodily fluids” may include, but are not limited to, blood, serum, plasma, saliva, cerebrospinal fluid, pleural fluid, tears, lactal duct fluid, lymph, sputum, urine, saliva, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin.

“Nucleic acid” or “nucleic acid sequence” as used herein refers to an oligonucleotide, nucleotide or polynucleotide, and fragments or portions thereof, which may be single or double stranded, and represent the sense or antisense strand. A nucleic acid may include DNA or RNA, and may be of natural or synthetic origin and may contain deoxyribonucleotides, ribonucleotides, or nucleotide analogs in any combination.

Non-limiting examples of polynucleotides include a gene or gene fragment, genomic DNA, exons, introns, mRNA, tRNA, rRNA, ribozymes, cDNA, recombinant polynucleotides, branched polynucleotides, plasmids, vectors, isolated DNA of any sequence, isolated RNA of any sequence, synthetic nucleic acid, nucleic acid probes and primers. Polynucleotides may be natural or synthetic. Polynucleotide may comprise modified nucleotides, such as methylated nucleotides and nucleotide analogs, uracyl, other sugars and linking groups such as fluororibose and thiolate, and nucleotide branches. A nucleic acid may be modified such as by conjugation, with a labeling component. Other types of modifications included in this definition are caps, substitution of one or more of the naturally occurring nucleotides with an analog, and introduction of chemical entities for attaching the polynucleotide to other molecules such as proteins, metal ions, labeling components, other polynucleotides or a solid support. Nucleic acid may include a nucleic acid that has been amplified (e.g., using polymerase chain reaction).

A fragment of a nucleic acid generally contains at least about 15, 20, 25, 30, 35, 40, 45, 50, 75, 100, 200, 300, 400, 500, 1000 nucleotides or more. Larger fragments are possible and may include about 2,000, 2,500, 3,000, 3,500, 4,000, 5,000 7,500, or 10,000 bases.

“Gene” as used herein refers to a DNA sequence that comprises control and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained.

Although a sequence of the nucleic acids may be shown in the form of DNA, a person of ordinary skill in the art recognizes that the corresponding RNA sequence will have a similar sequence with the thymine being replaced by uracil, i.e. “t” with “u”.

“Identity” and “identical” as used herein refer to a degree of identity between sequences. There may be partial identity or complete identity. A partially identical sequence is one that is less than 100% identical to another sequence. Preferably, partially identical sequences have an overall identity of at least 70% or at least 75%, more preferably at least 80% or at least 85%, most preferably at least 90% or at least 95% or at least 99%. Sequence identity determinations may be made for sequences which are not fully aligned. In such instances, the most related segments may be aligned for optimal sequence identity by and the overall sequence identity reduced by a penalty for gaps in the alignment.

“Hybridize” or “hybridization” as used herein refers to the pairing of substantially complementary nucleotide sequences (strands of nucleic acid) to form a duplex or heteroduplex through formation of hydrogen bonds between complementary base pairs. Hybridization and the strength of hybridization (i.e., the strength of the association between the nucleic acids) is influenced by such factors as the degree of complementary between the nucleic acids, stringency of the conditions involved, and the thermal melting point (Tₘ) of the formed hybrid. An oligonucleotide or polynucleotide (e.g., a probe or a primer) that is specific for a target nucleic acid will “hybridize” to the target nucleic acid under suitable conditions.

“Specific hybridization” as used herein refers to an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the Tₘ for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art.

“Stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

“Substantially complementary” as used herein refers to two sequences that hybridize under stringent hybridization conditions. The skilled artisan will understand that substantially complementary sequences need not hybridize along their entire length.

Oligonucleotides can be used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

“Oligonucleotide” as used herein refers to a molecule that has a sequence of nucleic acid bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group in this position. Oligonucleotides also may include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, reverse transcription, PCR, or a combination thereof. The oligonucleotide may be modified. For example, the oligonucleotide may be labeled with an agent that produces a detectable signal (e.g., a fluorophore).

“Primer” as used herein refers to an oligonucleotide that is capable of acting as a point of initiation of synthesis when placed under conditions in which primer extension is initiated (e.g., primer extension associated with an application such as PCR). The primer is complementary to a target nucleotide sequence and it hybridizes to a substantially complementary sequence in the target and leads to addition of nucleotides to the 3′end of the primer in the presence of a DNA or RNA polymerase. The 3′-nucleotide of the primer should generally be complementary to the target sequence at a corresponding nucleotide position for optimal expression and amplification. An oligonucleotide “primer” may occur naturally, as in a purified restriction digest or may be produced synthetically. The term “primer” as used herein includes all forms of primers that may be synthesized including peptide nucleic acid primers, locked nucleic acid primers, phosphorothioate modified primers, labeled primers, and the like.

Primers are typically between about 10 and about 100 nucleotides in length, preferably between about 15 and about 60 nucleotides in length, more preferably between about 20 and about 50 nucleotides in length, and most preferably between about 25 and about 40 nucleotides in length. In some embodiments, primers can be at least 8, at least 12, at least 16, at least 20, at least 25, at least 30, at least 35, at least 40, at least 45, at least 50, at least 55, at least 60 nucleotides in length. An optimal length for a particular primer application may be readily determined in the manner described in H. Erlich, PCR Technology, Principles and Application for DNA Amplification (1989).

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid.

Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be fully complementary to a target nucleic acid sequence or partially complementary. A probe may be used to detect the presence or absence of a target nucleic acid. Probes are typically at least about 10, 15, 20, 25, 30, 35, 40, 50, 60, 75, 100 nucleotides or more in length.

“Detecting” as used herein refers to determining the presence of a nucleic acid of interest in a sample or the presence of a protein of interest in a sample. Dection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label may be detected directly. In other cases, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluoresence, or chemiluminescence, or any other appropriate means.

“TaqMan® PCR detection system” as used herein refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

“Vector” as used herein refers to a recombinant DNA or RNA plasmid or virus that comprises a heterologous polynucleotide capable of being delivered to a target cell, either in vitro, in vivo or ex-vivo. The polynucleotide can comprise a sequence of interest and can be operably linked to another nucleic acid sequence such as promoter or enhancer and may control the transcription of the nucleic acid sequence of interest. As used herein, a vector need not be capable of replication in the ultimate target cell or subject. The term vector may include expression vector and cloning vector.

Suitable expression vectors are well-known in the art, and include vectors capable of expressing a polynucleotide operatively linked to a regulatory sequence, such as a promoter region that is capable of regulating expression of such DNA. Thus, an “expression vector” refers to a recombinant DNA or RNA construct, such as a plasmid, a phage, recombinant virus or other vector that, upon introduction into an appropriate host cell, results in expression of the inserted DNA. Appropriate expression vectors include those that are replicable in eukaryotic cells and/or prokaryotic cells and those that remain episomal or those which integrate into the host cell genome.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene, and the ability to enter and/or replicate in eukaryotic or prokaryotic cells. Additionally elements may also be included in the vector such as signal sequences, splice signals, as well as transcriptional promoters, enhancers, and termination signals.

Examples of suitable vectors include, but are not limited to plasmids pcDNA3, pHCMV/Zeo, pCR3.1, pEF I/His, pEMD/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.).

“Promoter” as used herein refers to a segment of DNA that controls transcription of polynucleotide to which it is operatively linked. Promoters, depending upon the nature of the regulation, may be constitutive or regulated. Exemplary eukaryotic promoters contemplated for use in the practice of the present invention include the SV40 early promoter, the cytomegalovirus (CMV) major immediate-early promoter, the mouse mammary tumor virus (MMTV) steroid-inducible promoter, Moloney murine leukemia virus (MMLV) promoter. Exemplary promoters suitable for use with prokaryotic hosts include T7 promoter, beta-lactamase promoter, lactose promoter systems, alkaline phosphatase promoter, a tryptophan (trp) promoter system, and hybrid promoters such as the tac promoter.

“Antibody” as used herein refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

“Specifically binds to a polypeptide” as used herein in the context of an antibody refers to binding of an antibody specifically to certain epitope of a polypeptide such that the antibody can distinguish between two proteins with and without such epitope.

### BRIEF DESCRIPTION OF THE DRAWINGS

FIG. 1A is a representative sequence trace of the 79INS BCR-ABL1 alternative splicing variant. The sequences are compared to an ABL1 kinase domain reference sequence. FIG. 1B is a schematic diagram and amino acid sequence alignment of ABL1 wild type (SEQ ID NO: 1), 35INS (SEQ ID NO: 2), 79INS (“35+44 ins”; SEQ ID NO: 3), 84INS (SEQ ID NO: 43), and 231INS (SEQ ID NO: 5) splicing variants. The previously described 35INS mutation is also included for comparison. Sequence differences caused by frameshift mutations are shown. Premature stop codons are indicated by asterisks, and the relative position of T315 is shown.

FIG. 2 shows the relative frequency of individual BCR-ABL kinase domain mutations detected in a group of 245 patients, 219 of which have CML and 26 of which have Ph-positive acute lymphoblastic leukemia. The numbering of amino acids is based on the Abl protein variant B (which includes ABL exon 1b but not exon 1a). At some positions, 2 or 3 possible mutants with different amino acids are possible. The percentage of patients with each mutation specified is shown in the right most column.

FIG. 3 shows the cDNA sequence of the human ABL gene (FIG. 3A; SEQ ID NO: 6) and the encoded ABL protein (FIG. 3B; SEQ ID NO: 7) as provided in GenBank Accession No.: M14752.

FIG. 4 shows the complete cDNA sequence of the human ABL gene, including exons 1a and 2-10 as provided in GenBank Accession No.: U07563.1 (SEQ ID NO: 8).

### DETAILED DESCRIPTION OF THE INVENTION

### BCR-ABL1

The inventions described herein include polynucleotides which encode all or portions of the splice variants in the BCR-Abl gene product, cells that express all or portions of those splice variants, and proteins encoded by those splice variants. Recombinant cells expressing the truncated BCR-ABL protein with an active kinase domain are useful for identifying drug candidates for treating CML. Methods for predicting likelihood for responsiveness to kinase inhibitor therapy are included along with methods, compositions and reagents for detecting the splice variants.

Variants of the bcr-abl1 mRNA

Several splice variants of bcr-abl1 mRNA have been reported. Many of the known sequences are full length cDNA sequences and some are partial cDNA sequences. For example bcr-abl1 mRNA sequences include: NCBI GenBank accession numbers: EU216072, EU216071, EU216070, EU216069, EU216068, EU216067, EU216066, EU216065, EU216064, EU216063, EU216062, EU216061, EU216060, EU216059, EU216058, EU236680, DQ912590, DQ912589, DQ912588, DQ898315, DQ898314, DQ898313, EF423615, EF158045, S72479, S72478, AY789120, AB069693, AF487522, AF113911, AF251769, M30829, M30832, M17542, M15025, and M17541.

Additionally, BCR-ABL1 variant protein sequences have been reported, for example NCBI protein database accession numbers: ABX82708, ABX82702, AAA35594.

CML patients undergoing BCR-ABL1 tyrosine kinase inhibitor therapy may develop resistance to the therapy. The resistance in patients has been associated with mutant splice variant forms of BCR-ABL1. For example, an insertion/truncation mutant of BCR-ABL1 in a CML patient resistant to imatinib has been reported which results from a 35 base insertion of abl intron 8 into the junction between exons 8 and 9 of the bcr-abl mRNA due to alternate splicing (35 INS variant; see U.S. patent application Ser. No. 12/472,319).

### 79INS Splice Variant

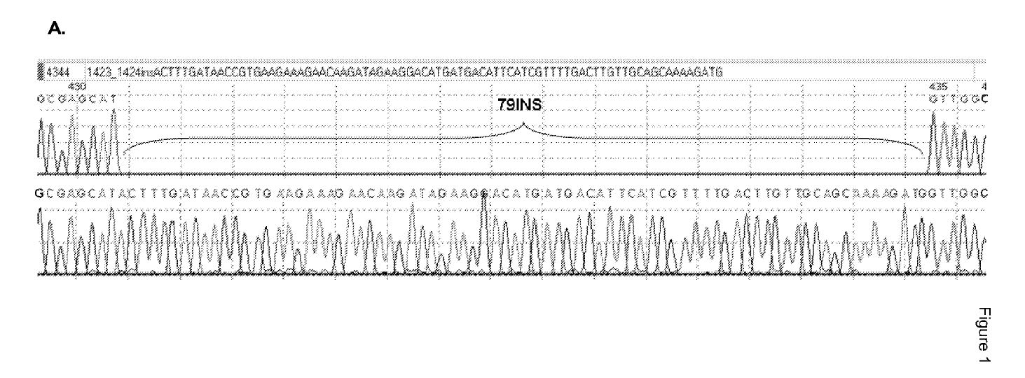

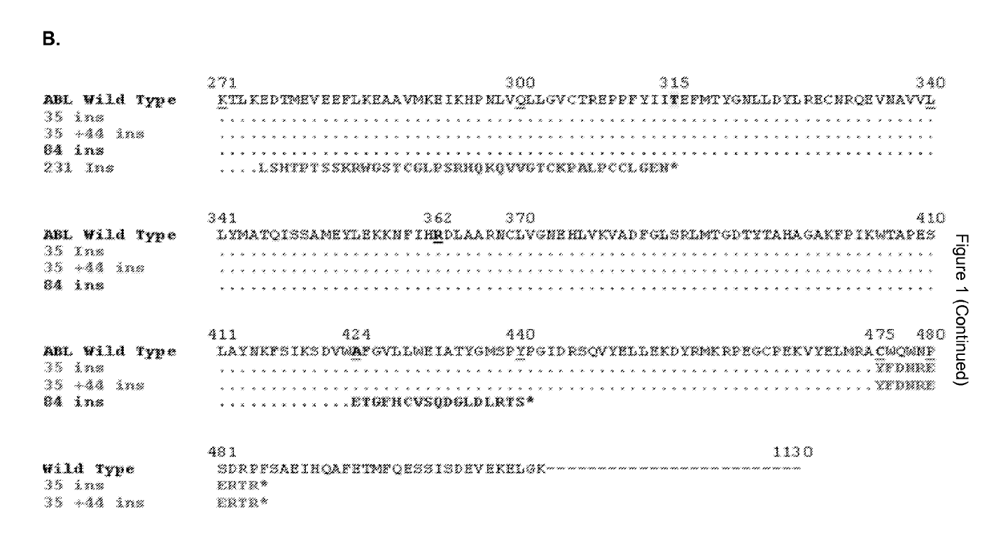

This splice mutation resulted from the insertion of 79 nucleotides into the ABL1 exon 8-9 junction. The inserted sequence contains nucleotides from regions of intron 8, located 120 bp apart: the 35-nucleotide sequence previously described, and an additional 44-nucleotide segment downstream from 35INS. The combined 79-nucleotide insertion splice mutant showed the same protein change as 35INS (p C475YfsX11) (FIG. 1). Thus, the intron 8 nucleotide sequence inserted by the alternate splicing is:

(SEQ ID NO: 9)
5′- actttgataa ccgtgaagaa agaacaagat agaaggtgag ctgtttggct tagtaatttt
ctacacctac tagagcggg -3′

Which results in a non-native ABL1 C-terminus having the following amino acid sequence:

| | |
|---|---|
| - - - YFDNREERTR | (SEQ ID NO: 10) |

Included in the invention are oligonucleotides, primers or probes which are designed to be complementary to some or all of the above sequence or to be complementary to some or all of the above sequence and to some adjoining sequence (not shown) in the mRNA (i.e., a junction sequence). Such oligonucleotides primers or probes can be readily designed so as to hybridize under stringent conditions to 79INS splice variant but not hybridize to the regular BCR/Abl mRNA or to any other known BCR/Abl insertion splice variants.

### 84INS Splice Variant

This splice mutation comprised an 84-nucleotide sequence from intron 7 inserted into the ABL1 exon7-8 junction, also causing a frameshift and protein truncation (p A424EfsX18) (FIG. 1). The non-native ABL1 C-terminus created by the 84INS splice variant is:

| | |
|---|---|
| - - - ETGFHCVSQDGLDLRTS | (SEQ ID NO: 11) |

This patient also carried a point mutation at nucleotide 926 (C to G; P309R) in ABL1 exon 6. Included in the invention are oligonucleotides, primers or probes which are designed to be complementary to some or the 84 nucleotides from intron 7 that are present in the mRNA (or cDNA) or which are designed to be complementary to some or all of the 84 nucleotides and to some adjoining sequence in the mRNA (i.e., a junction sequence). Such probes can be readily designed so as to hybridize under stringent conditions to 84INS splice variant but not hybridize to the regular BCR/Abl mRNA or any other known BCR/Abl insertion splice variants.

### 231INS Splice Variant

This splice mutation derived from a 231-nucleotide sequence from intron 4 retained in the ABL1 exon 4-5 junction, and was first noticed because of an unusually large PCR product (1100 bp+) on gel electrophoresis, with corresponding absence of the regular BCR-ABL1 transcript (863 bp). The 231-nucleotide insertion added 40 intron-encoded amino acids after K274, leading to a frameshift and early termination of BCR-ABL1 in the p-loop (p E275LfsX41) (FIG. 1). The non-native ABL1 C-terminus created by the 231INS splice variant (i.e., the 40 intron-encoded amino acids) is:

(SEQ ID NO: 12)
- - - LSHTPTSSKRWGSTCGLPSRHQKQVVGTCKPALPCCLGEN

This patient also showed an A to G point mutation at nucleotide 899 of exon 5 (Q300R) in <60% of total BCR-ABL1 transcripts. Included in the invention are oligonucleotides, primers or probes which are designed to be complementary to some or the 231 nucleotides from intron 4 that are present in the mRNA (or cDNA) or which are designed to be complementary to some or all of the 231 nucleotides and to some adjoining sequence in the mRNA (i.e., a junction sequence). Such probes can be readily designed so as to hybridize under stringent conditions to 231INS splice variant but not hybridize to the regular BCR/Abl mRNA or any other known BCR/Abl insertion splice variants. The 40 intron-encoded amino acids produced by the splice variant protein product may be detected by antibodies that are specific for the intron encoded sequence (or specific for a junction sequence including amino acids of the intron encoded sequence with amino acids upstream (5′) or downstream (3′) of that sequence.

### Mutations in the ABL Kinase Domain:

CML patients undergoing tyrosine kinase inhibitor therapy (such as, imatinib, nilotinib, dasatinib, Bosutinib (SKI-606) and Aurora kinase inhibitor VX-680) may develop resistance to such inhibitors. Several underlying mechanisms of resistance to kinase inhibitors have been identified. One major cause is the presence of point mutations within the ABL kinase domain of BCR-ABL. In one embodiment, such mutations inhibit the ability of imatinib to bind to BCR-ABL by altering the binding sites or preventing the kinase domain from assuming the inactive conformation required for imatinib binding (O'Hare et al. Blood. 2007; 110: 2242-2249). Point mutations develop in approximately 35% to 70% of patients displaying resistance to imatinib, either spontaneously or through the evolutionary pressure of imatinib (Branford et al. Blood. 2003; 102: 276-283).

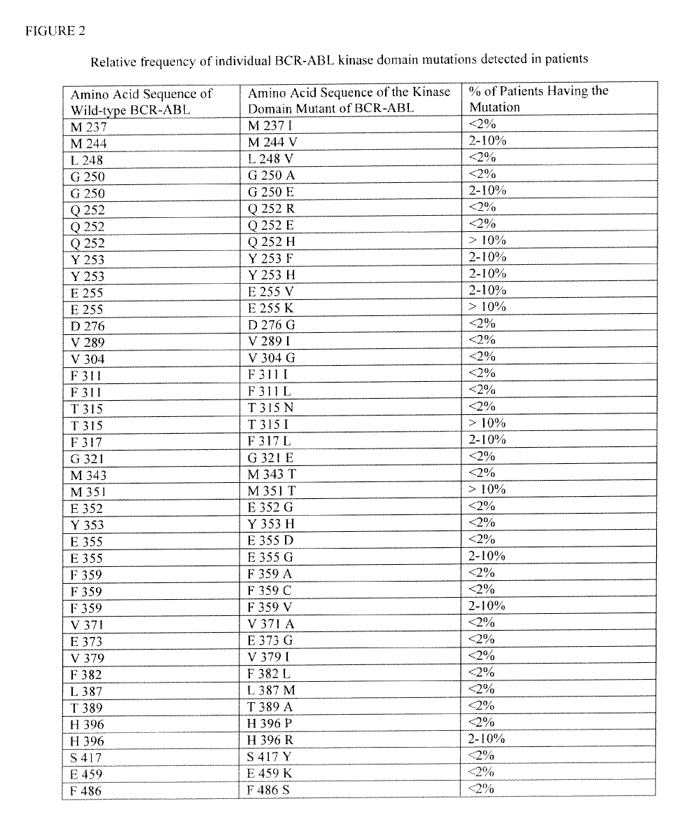

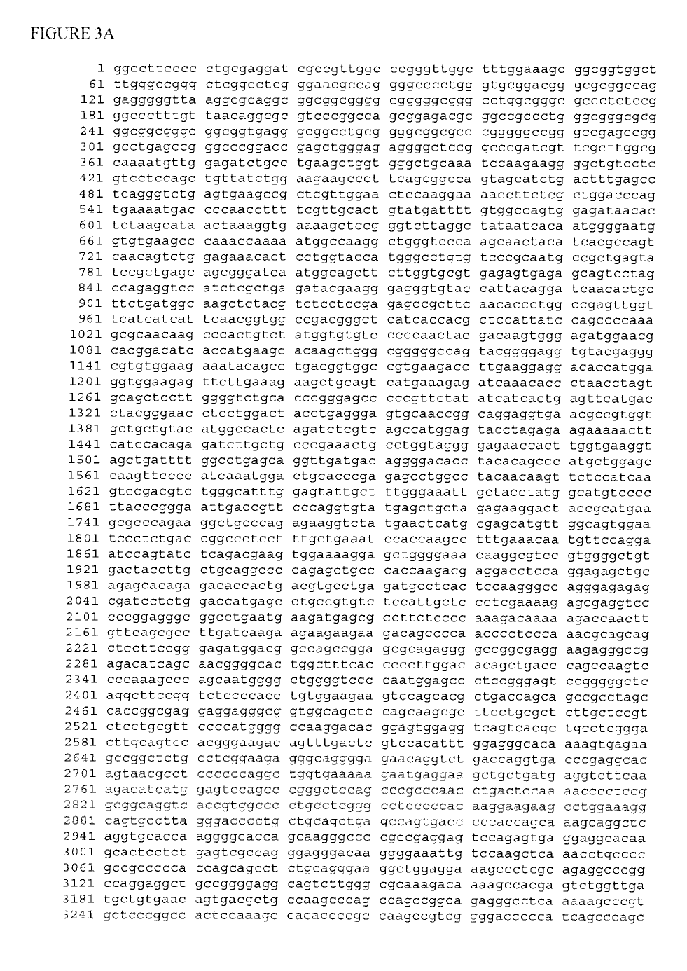

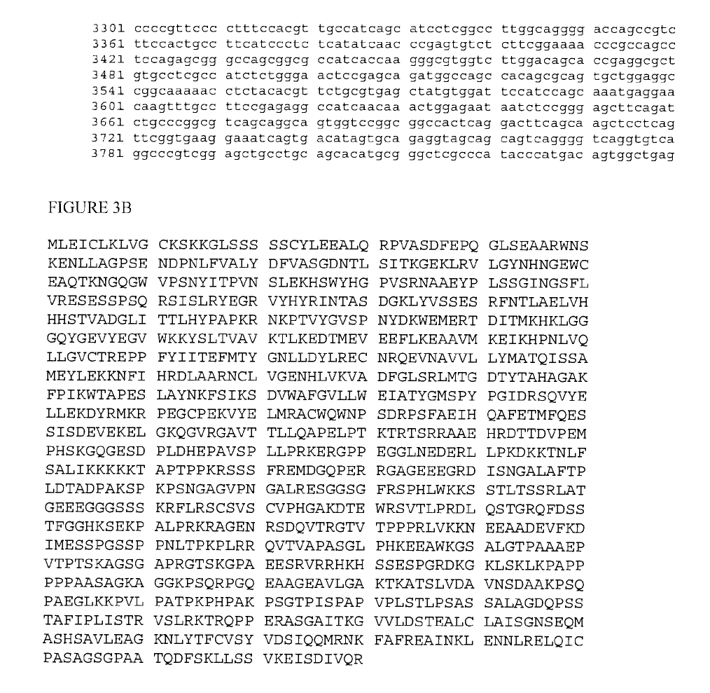

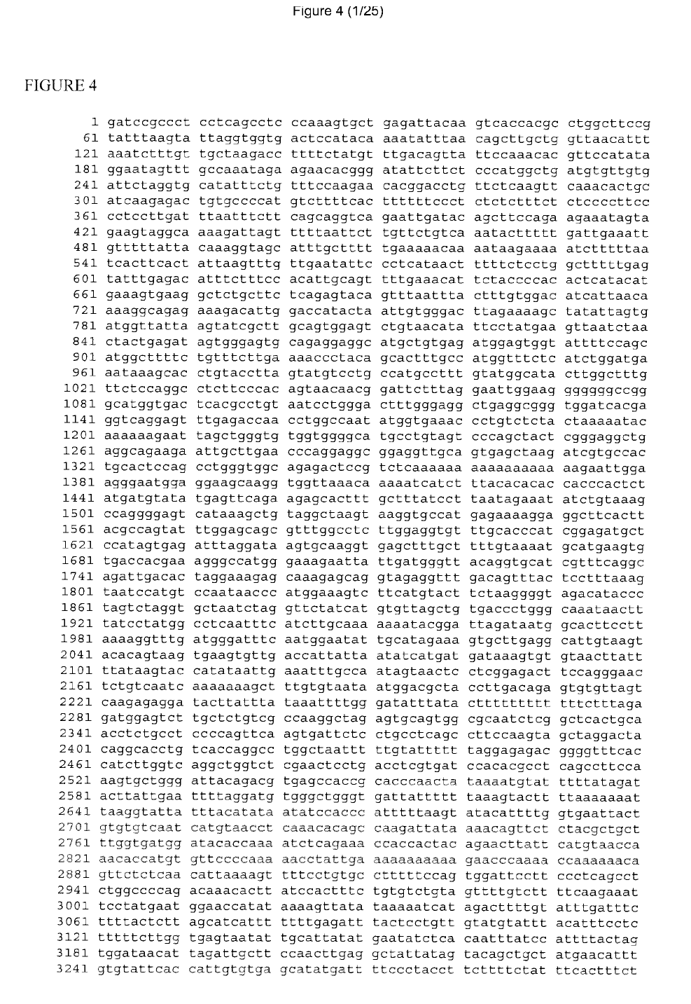

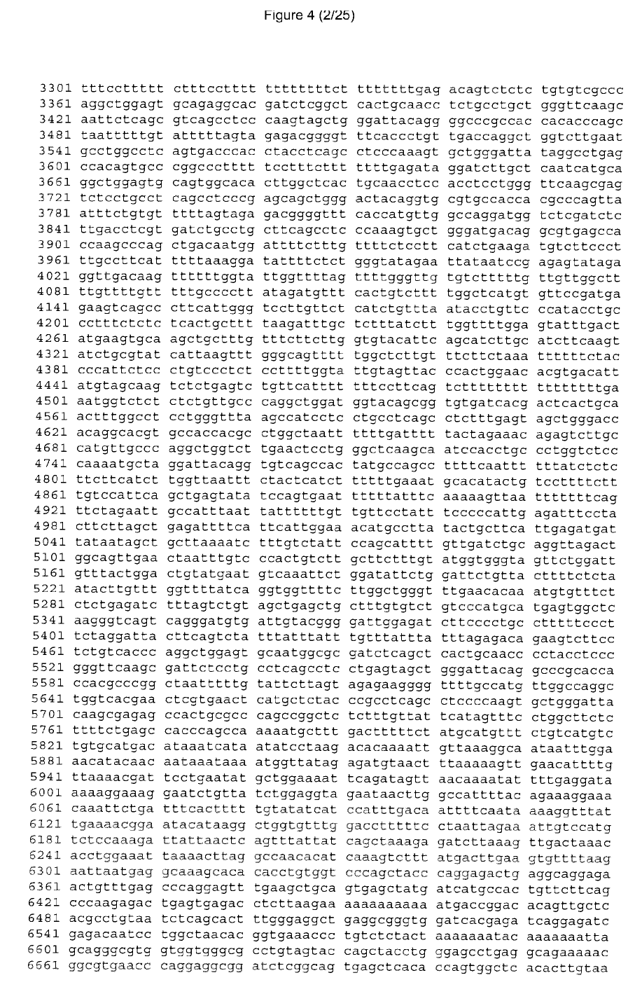

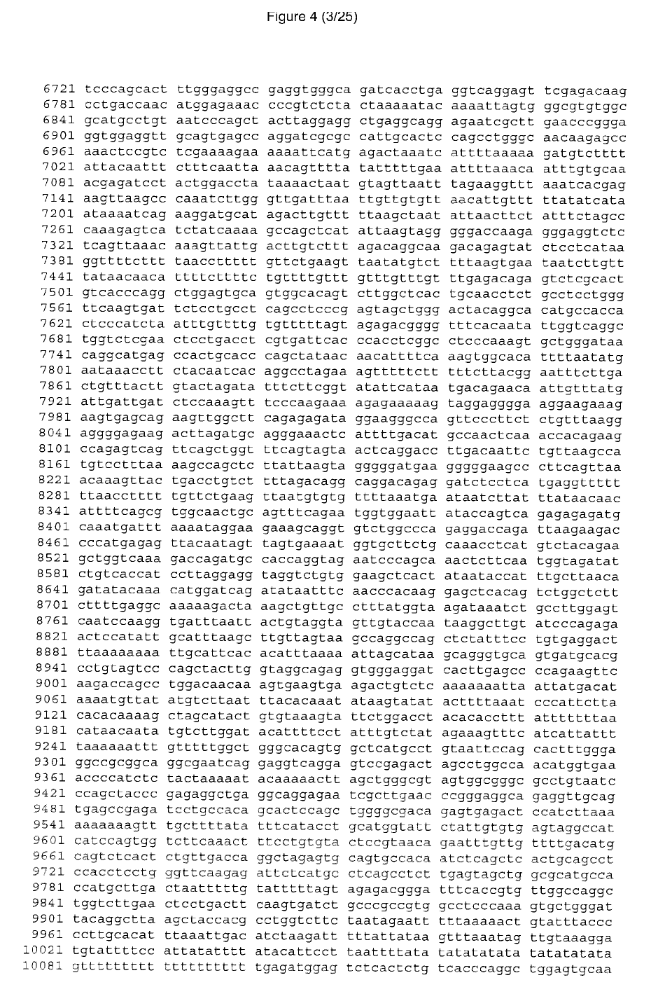

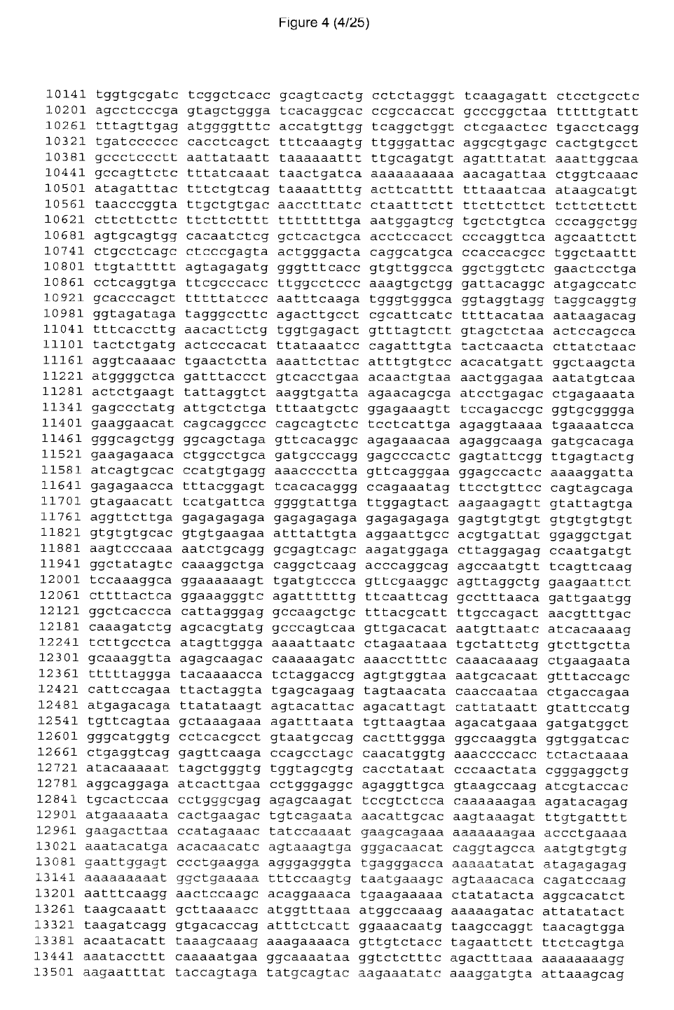

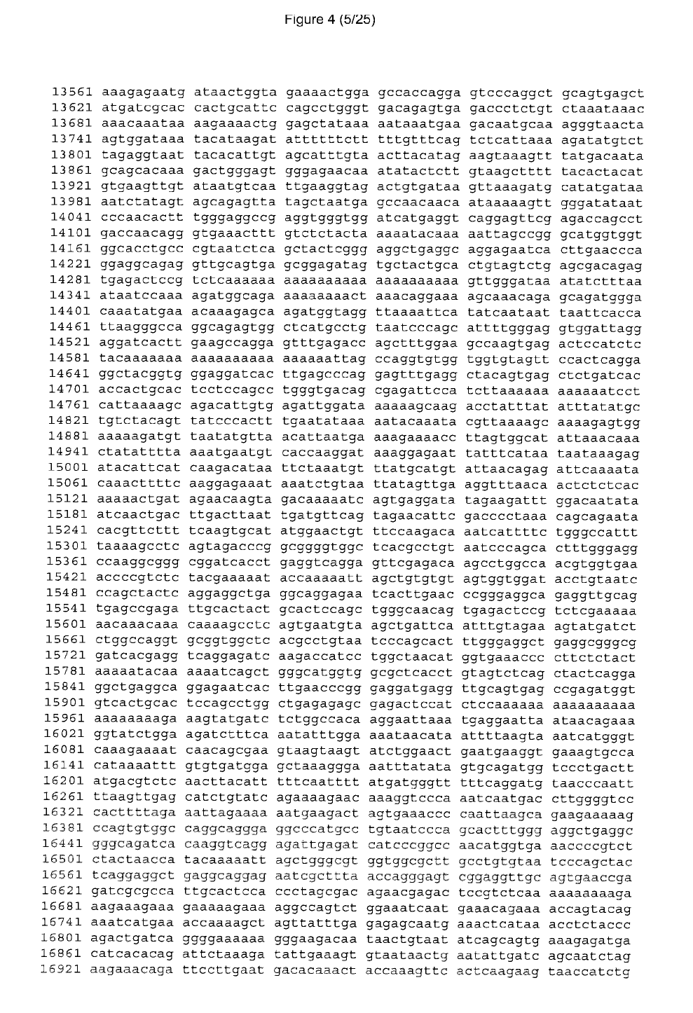

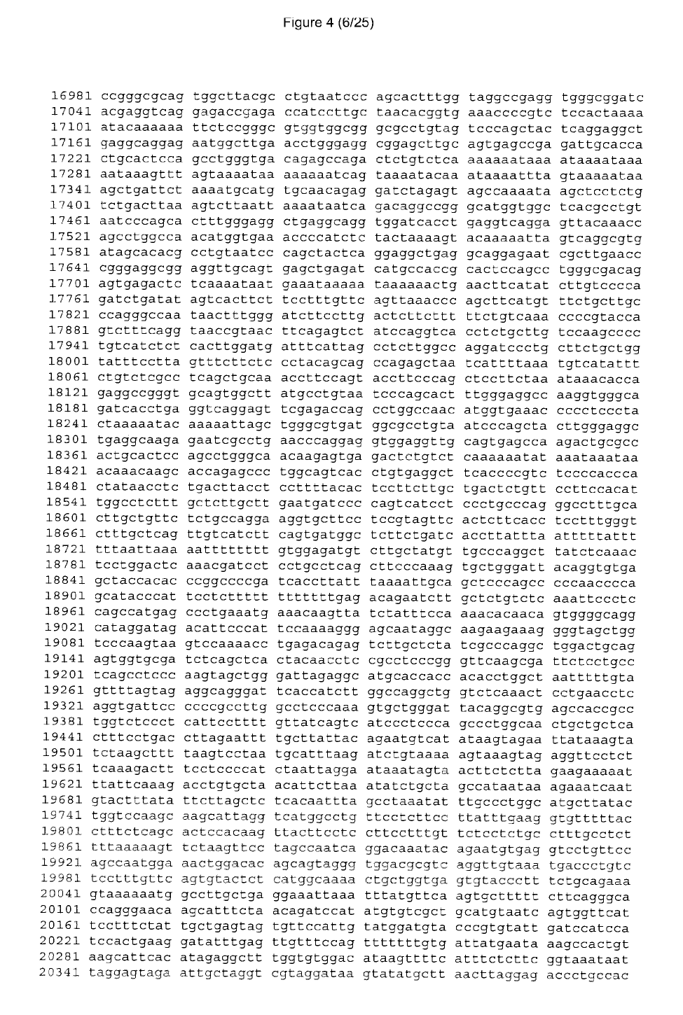

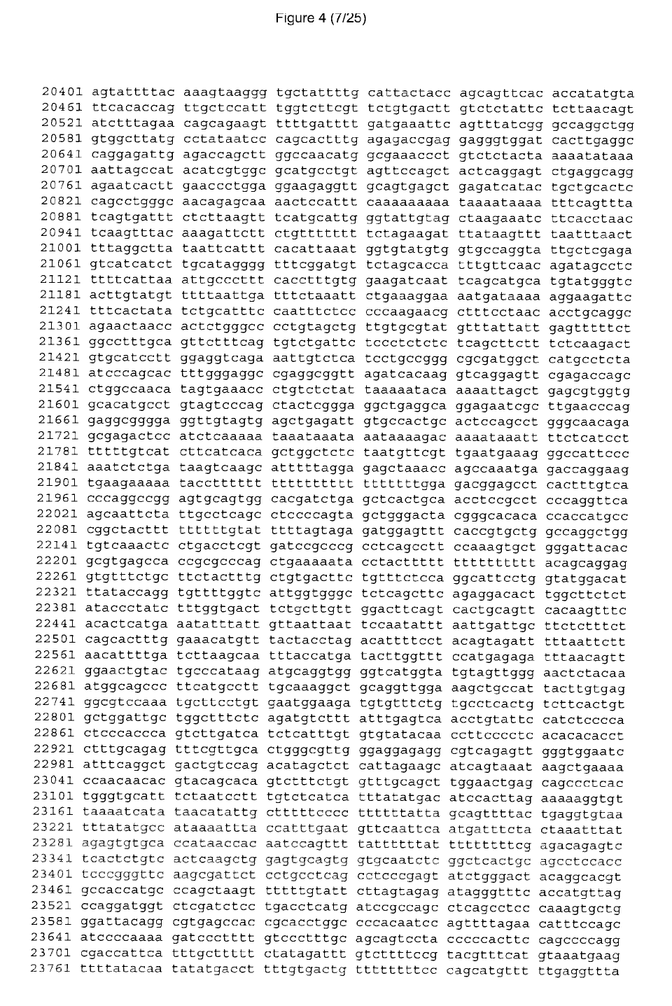

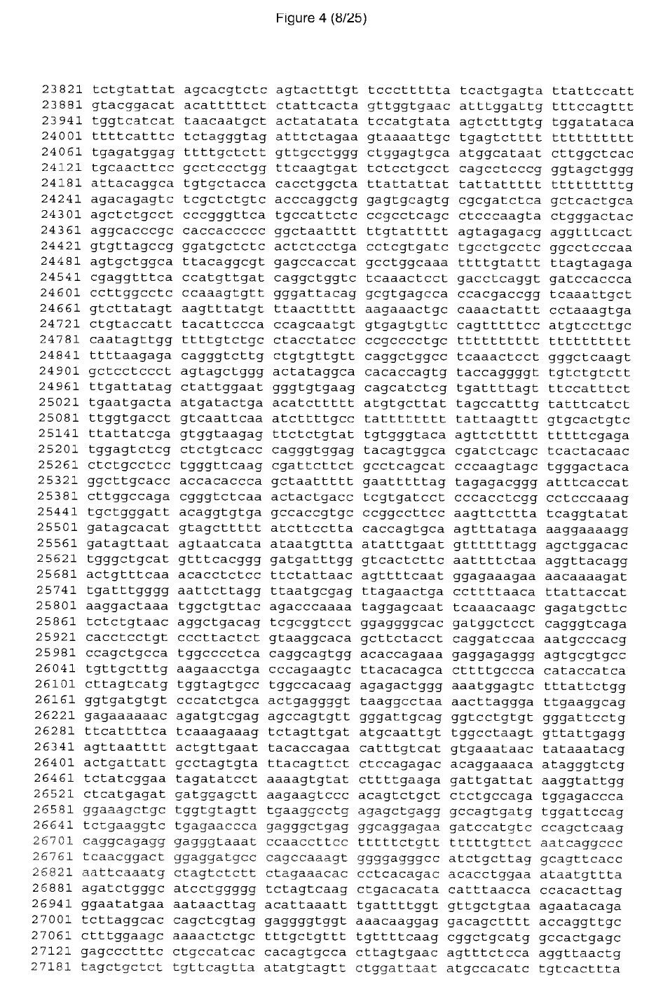

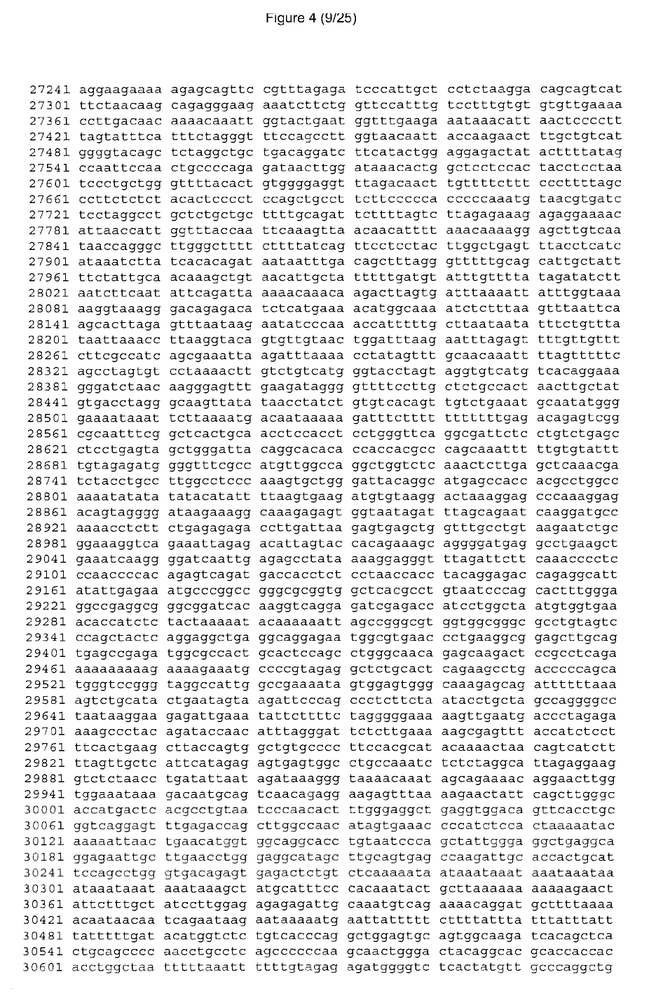

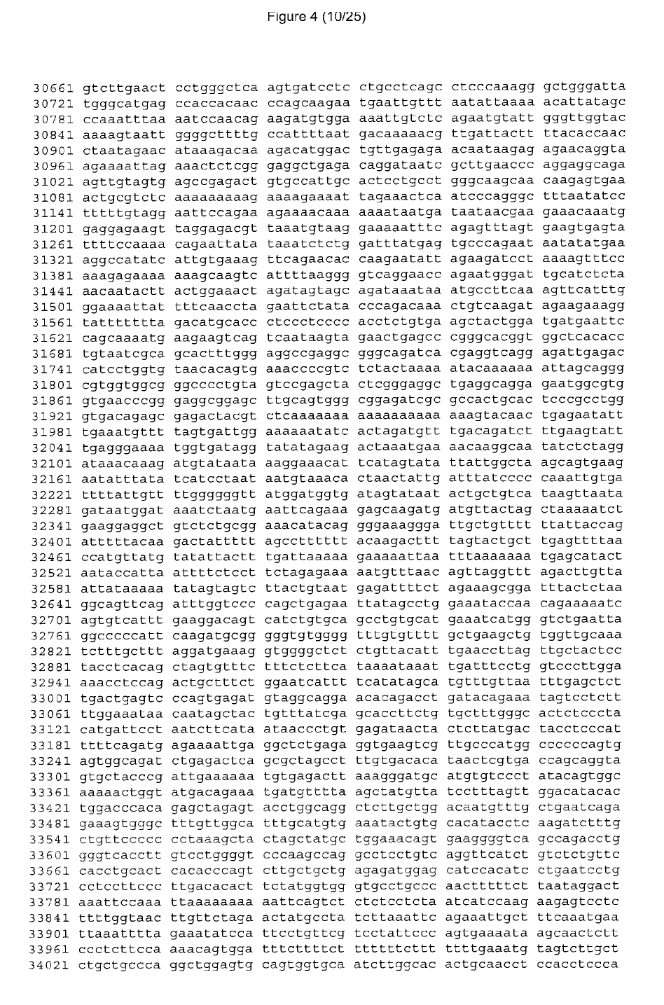

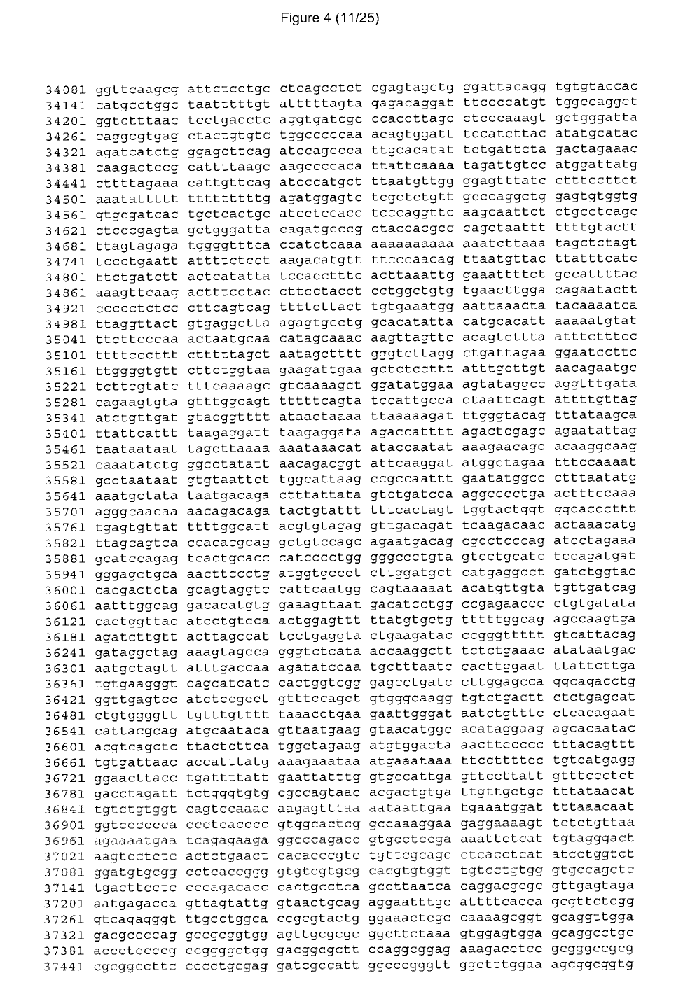

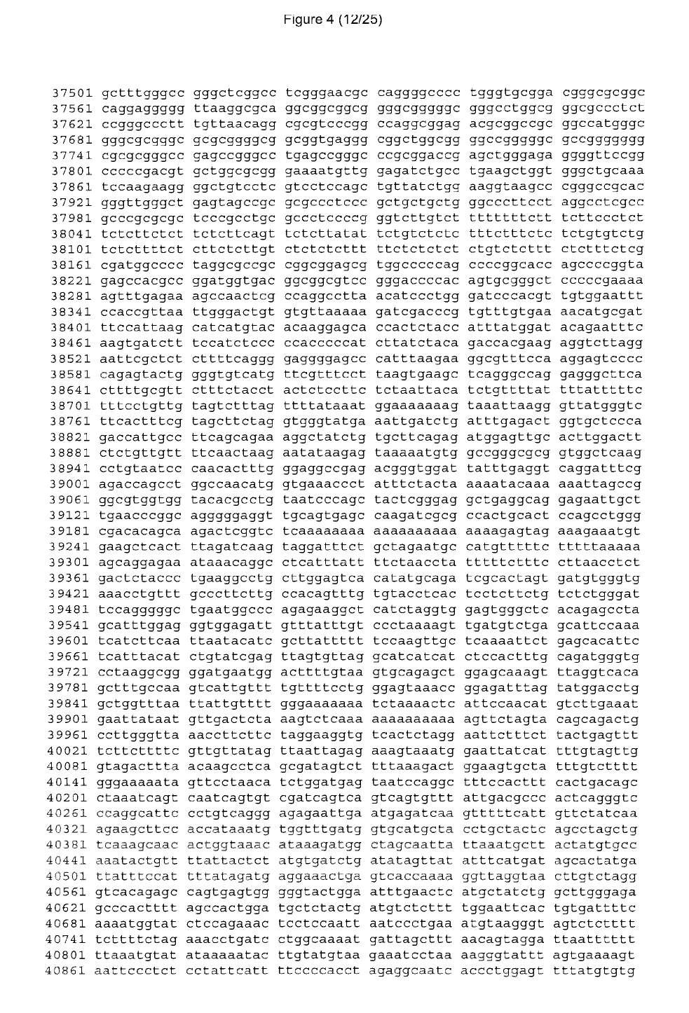

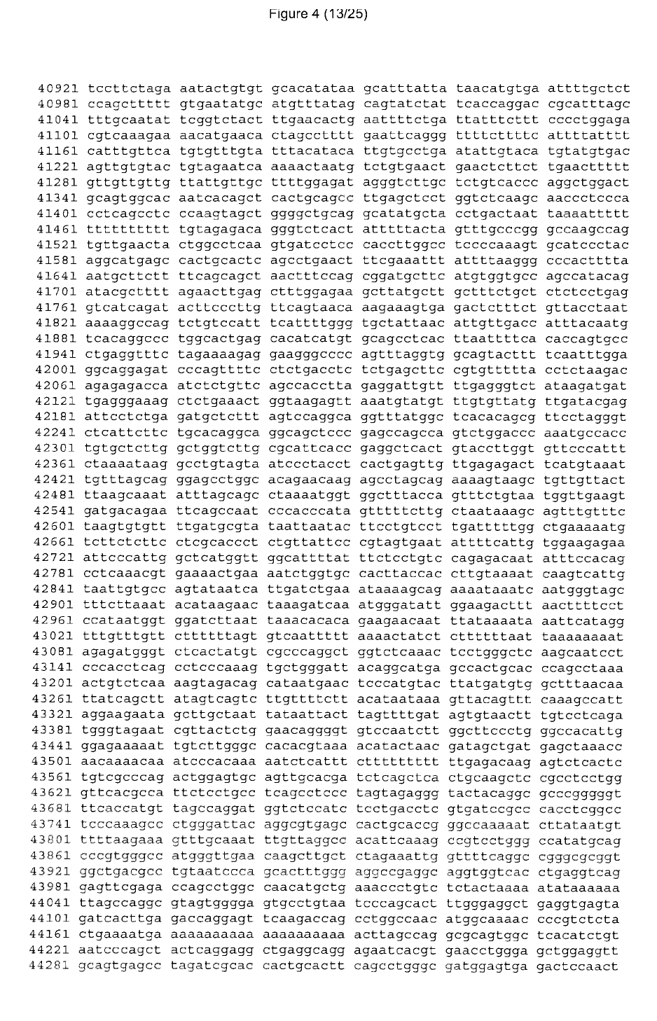

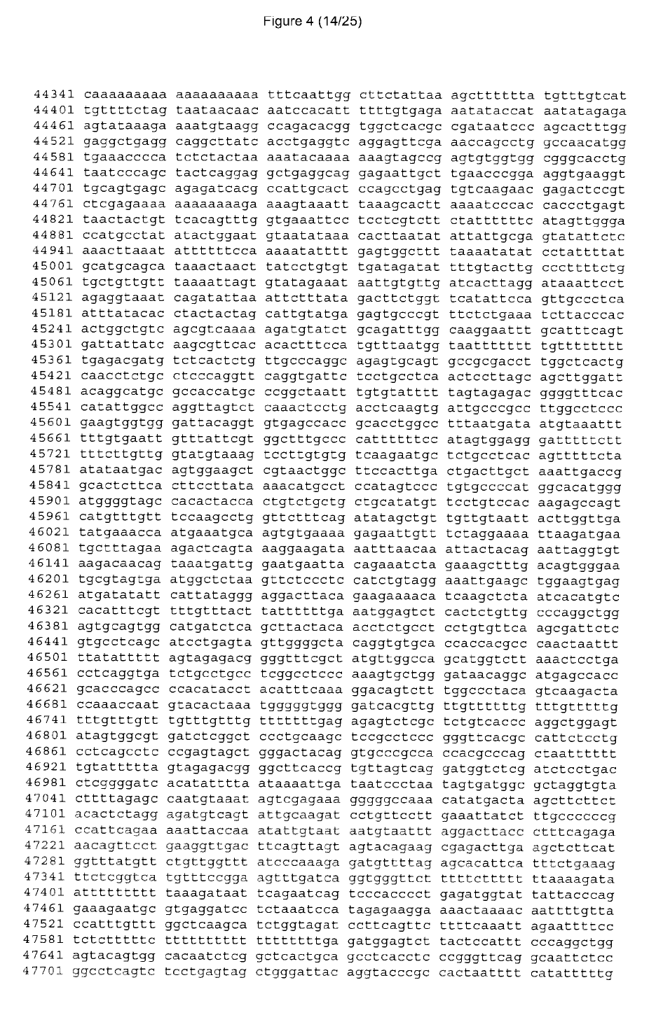

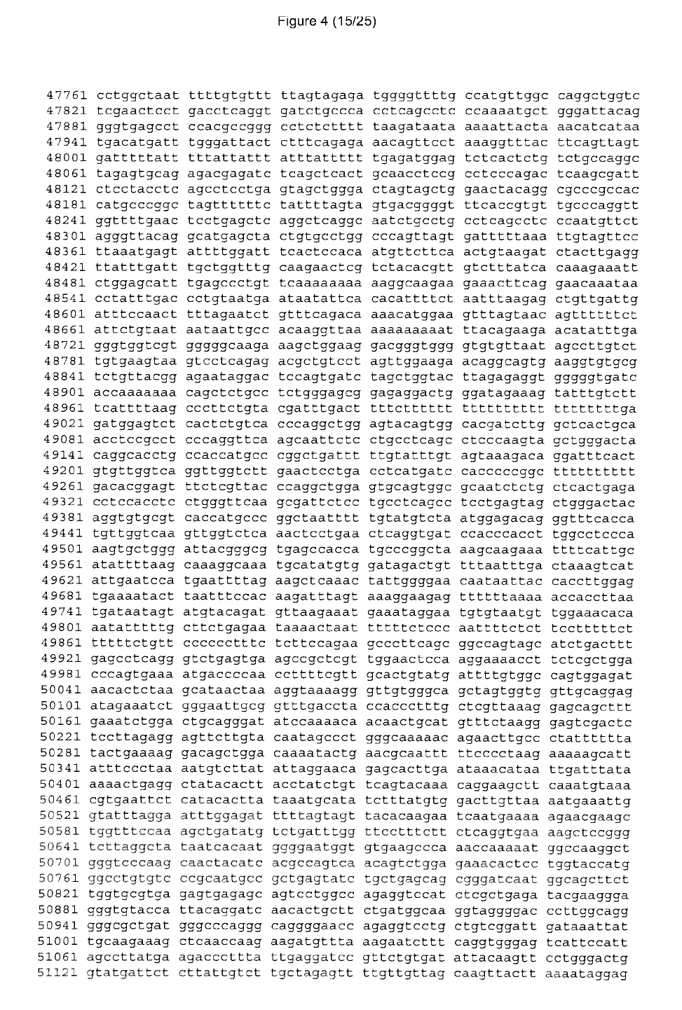

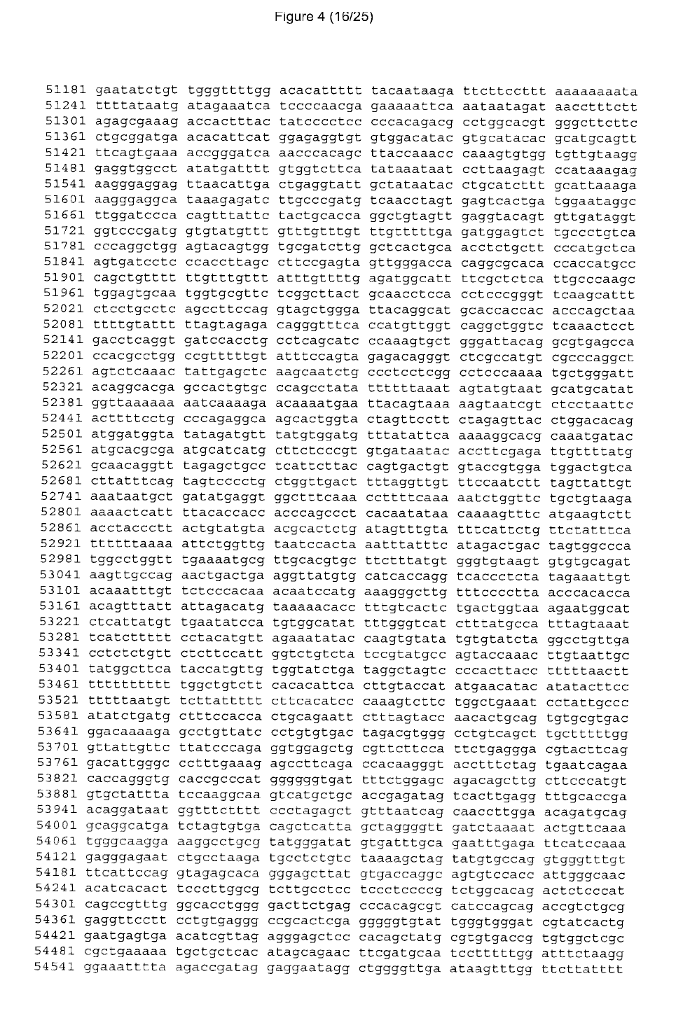

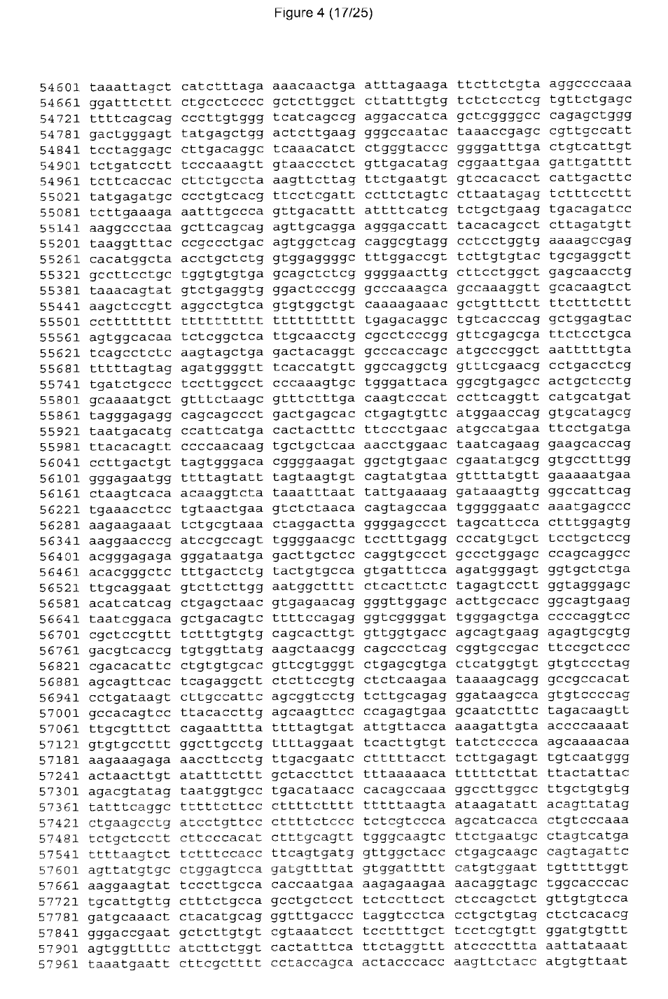

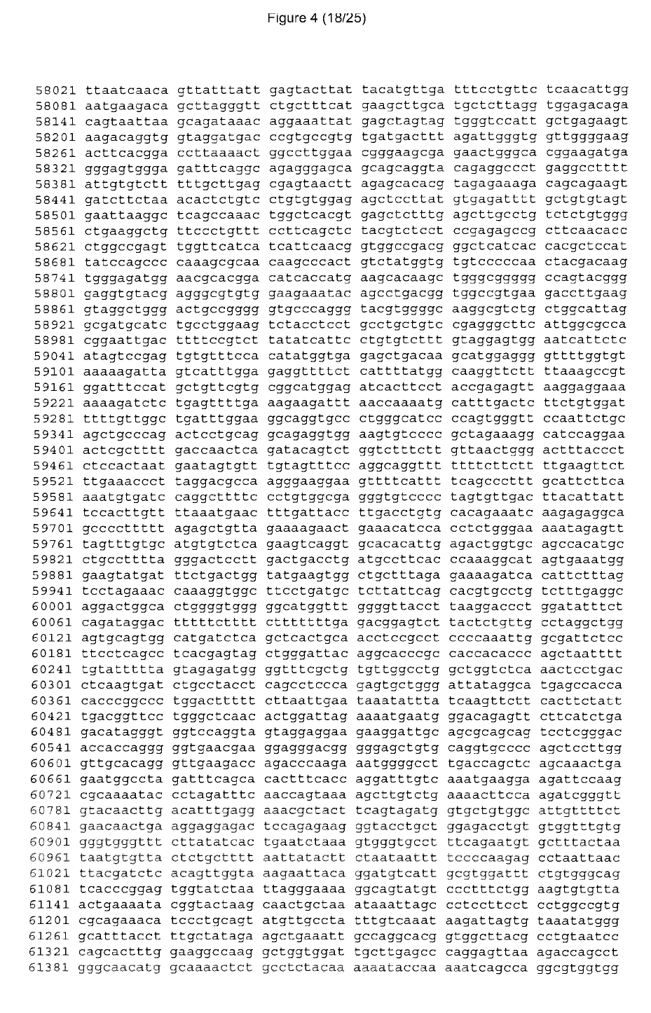

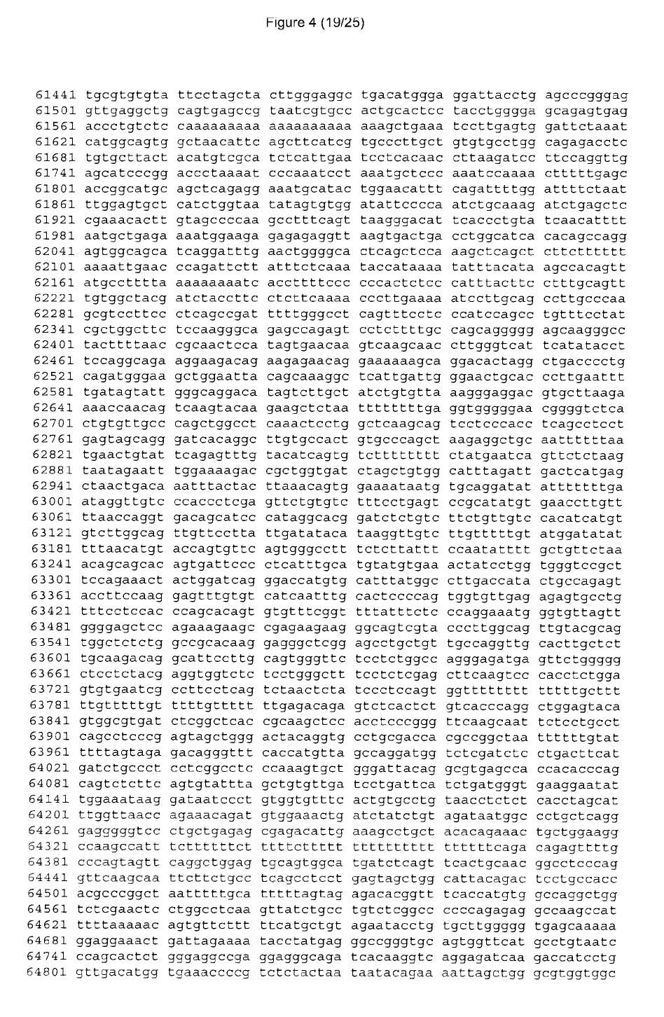

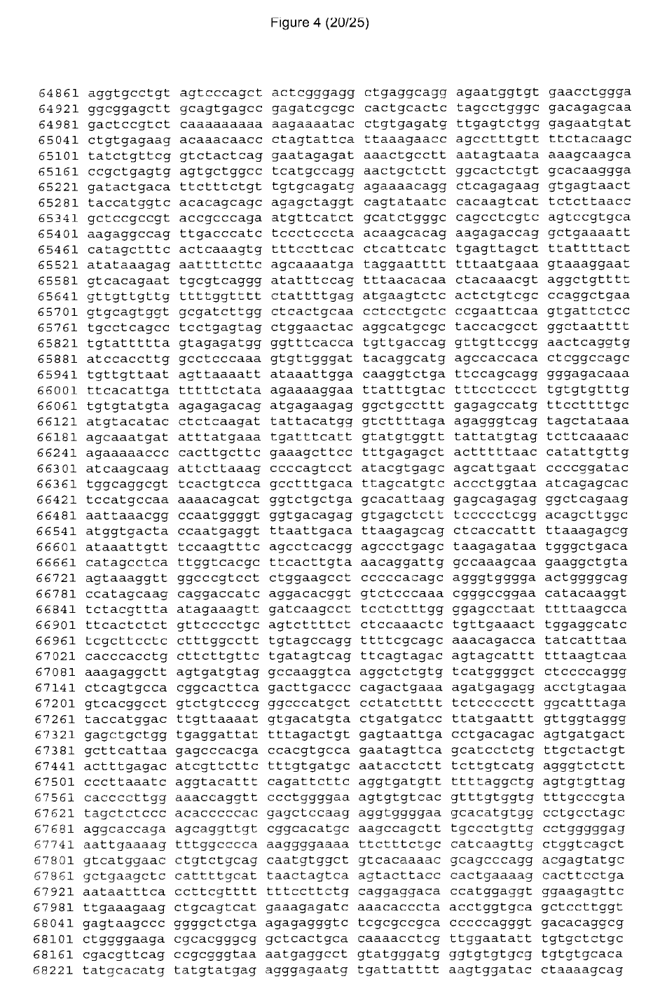

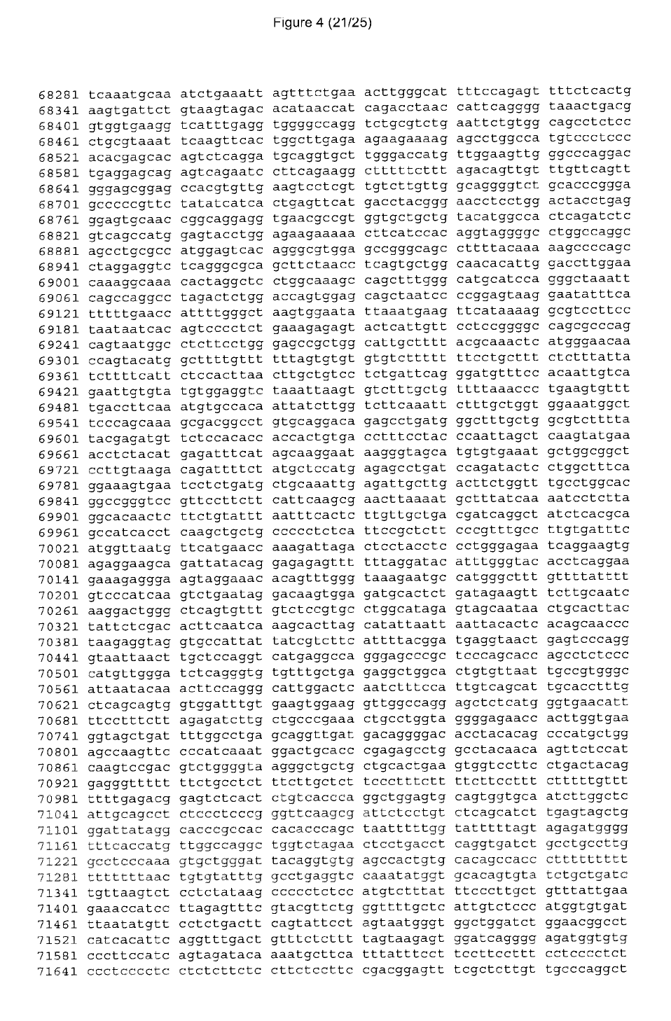

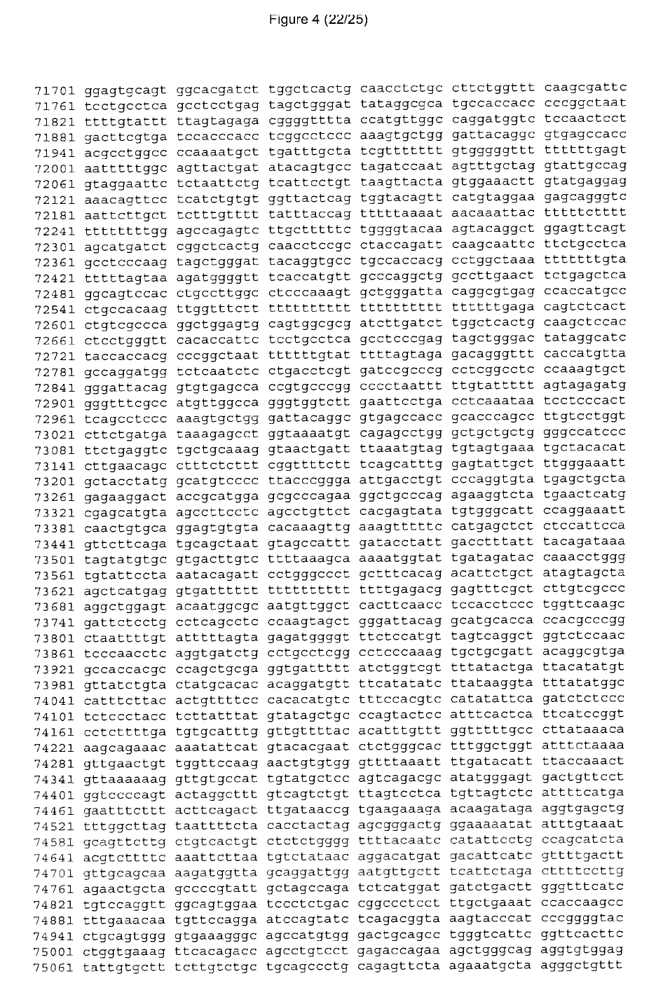

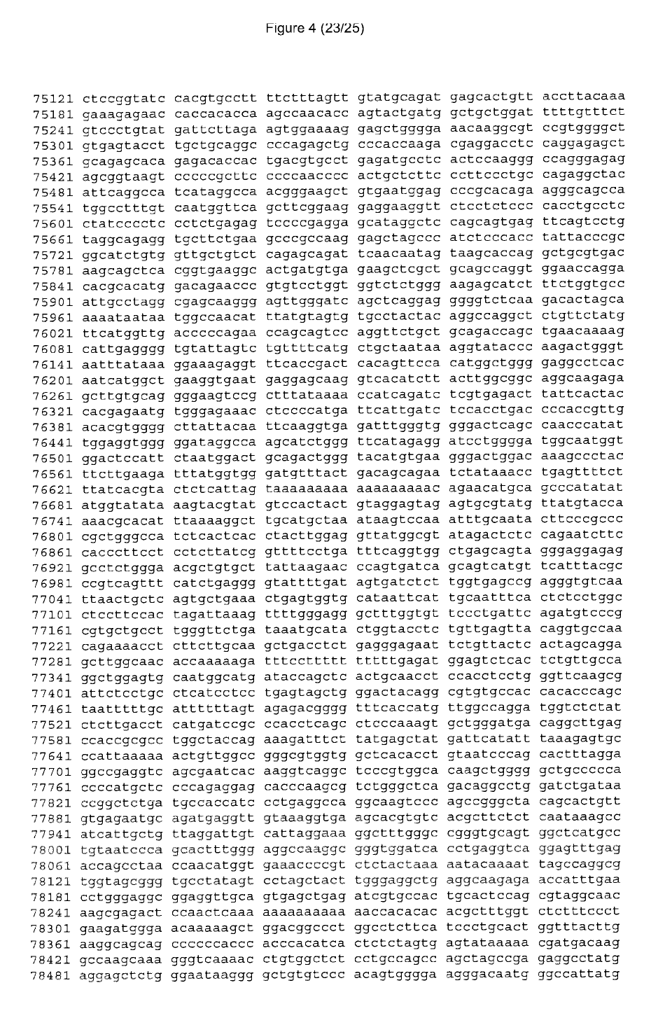

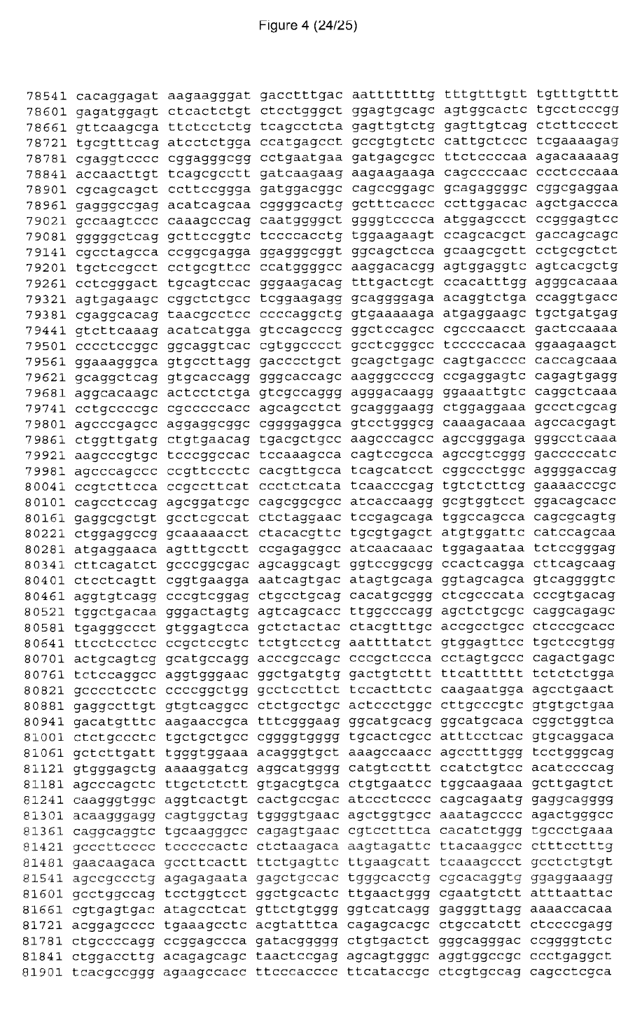

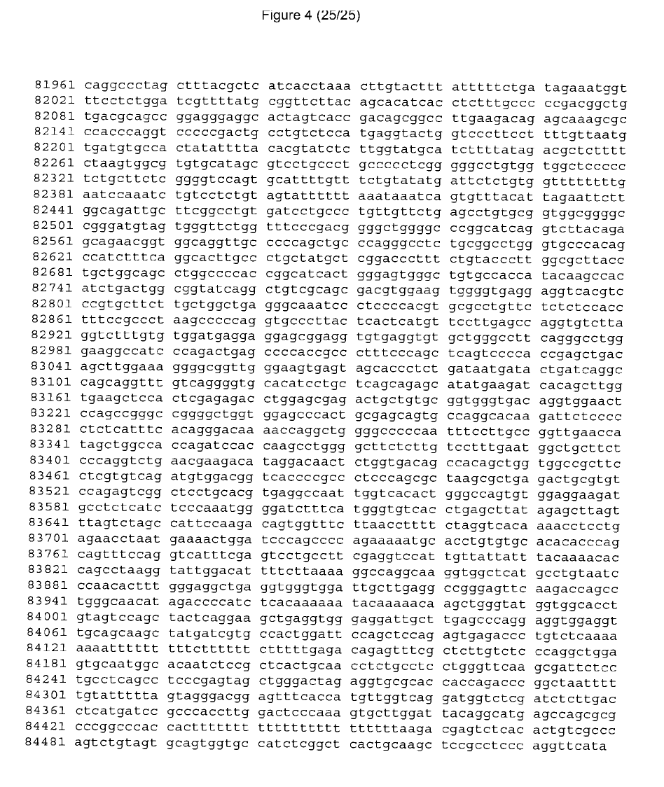

More than 40 distinct resistance-conferring mutations have been detected; the majority fall within four regions of the kinase domain: the ATP-binding loop (P-loop) of the ABL kinase domain, the contact site, the SHY binding site (activation loop), and the catalytic domain (Hughes et al. Blood. 2006; 108: 28-37). A list of such mutations are shown in FIG. 2 and incorporated herein by reference. Approximately 85% of all imatinib-resistant mutations are associated with amino acid substitutions at just seven residues (P-loop: M244V, G250E, Y253F/H and E255K/V; contact site: T315I; and catalytic domain: M351T and F359V). The most frequently mutated region of BCR-ABL is the P-loop, accounting for 36% to 48% of all mutations.

The importance of P-loop mutations is further underlined by in vitro evidence suggesting that these mutations are more oncogenic with respect to un-mutated BCR-ABL as well as other mutated variants. In various biological assays, P-loop mutants Y253F and E255K exhibited an increased transformation potency relative to un-mutated BCR-ABL. Overall, the relative transformation potencies of various mutations were found to be as follows: Y253F>E255K>native BCR-ABL≧T315I>H396P>M351T. Transformation potency also correlated with intrinsic BCR-ABL kinase activity in this study.

In some embodiments, CML patients undergoing kinase inhibitor therapy may develop two kinds of mutations: a) an insertion/truncation mutant of BCR-ABL due to alternate splicing and b) one or more point mutations in the kinase domain of Abl.

In preferred embodiments, the alternate splice variant of bcr-abl mRNA can be detected simultaneously with the detection of mutations in abl portion of bcr-abl mRNA. In another embodiment, the mutations in the abl portion of bcr-abl mRNA can be detected separately. Several methods are known in the art for detection of the presence or absence of such mutations. Non limiting examples include, DNA sequencing, detection by hybridization of a detectably labeled probe, detection by size, allele specific PCR, ligation amplification reaction (LAR), detection by oligonucleotide arrays.

### Biological Sample Collection and Preparation

Sample:

Samples, for use in the methods of the present invention, may be obtained from an individual who is suspected of having a disease, e.g. CML, or a genetic abnormality, or who has been diagnosed with CML. Samples may also be obtained from a healthy individual who is assumed of having no disease, e.g. CML, or genetic abnormality. Additionally, the sample may be obtained from CML patients undergoing kinase inhibitor therapy or from CML patients not undergoing kinase inhibitor therapy.

Sample Collection:

Methods of obtaining samples are well known to those of skill in the art and include, but are not limited to, aspirations, tissue sections, drawing of blood or other fluids, surgical or needle biopsies, collection of paraffin embedded tissue, collection of body fluids, collection of stool, urine, buccal swab and the like.

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70 degrees centigrade until thawed and used. “Fresh” plasma or serum should be refrigerated or maintained on ice until used. Exemplary methods of preparation are described below.

DNA/RNA/Protein Purification:

Polynucleotides (e.g., DNA or RNA) or polypeptides may be isolated from the sample according to any methods well known to those of skill in the art. If necessary, the sample may be collected or concentrated by centrifugation and the like. The sample may be subjected to lysis, such as by treatments with enzymes, heat, surfactants, ultrasonication, or a combination thereof. The lysis treatment is performed in order to obtain a sufficient amount of nucleic acid or polypeptide. The sample may be subjected to liquid chromatography to partially purify the nucleic acid or polypeptide. In some embodiments, the whole cell lysates or tissue homogenate may used as source of nucleic acid or polypeptide without further isolation and purification.

Suitable DNA isolation methods include phenol and chloroform extraction, see Sambrook, et al., Molecular Cloning: A Laboratory Manual (1989), Second Edition, Cold Spring Harbor Press, Plainview, N.Y.

Numerous commercial kits also yield suitable DNA including, but not limited to, QIAamp™ mini blood kit, Agencourt Genfind™, Roche Cobas® Roche MagNA Pure® or phenol: chloroform extraction using Eppendorf Phase Lock Gels®. Total DNA (e.g., genomic, mitochondrial, microbial, viral,) can be purified from any biological sample such as whole blood, plasma, serum, buffy coat, bone marrow, other body fluids, lymphocytes, cultured cells, tissue, and forensic specimens using commercially available kits e.g., QIAamp DNA and QIAamp DNA Blood mini kits from Qiagen.

In another embodiment, the polynucleotide may be mRNA or cDNA generated from mRNA or total RNA. RNA is isolated from cells or tissue samples using standard techniques, see, e.g., Sambrook, et al., Molecular Cloning: A Laboratory Manual, Second Edition (1989), Cold Spring Harbor Press, Plainview, N.Y. In addition, reagents and kits for isolating RNA from any biological sample such as whole blood, plasma, serum, buffy coat, bone marrow, other body fluids, lymphocytes, cultured cells, tissue, and forensic specimens are commercially available e.g., RNeasy Protect Mini kit, RNeasy Protect Cell Mini kit, QIAamp RNA Blood Mini kit, RNeasy Protect Saliva Mini kit, Paxgene Blood RNA kit from Qiagen; MELT™, RNaqueous®, ToTALLY RNA™, RiboPure™-Blood, Poly(A)Purist™ from Applied Biosystems; TRIZOL® reagent, Dynabeads® mRNA direct kit from Invitrogen.

In some embodiments, the nucleic acid is isolated from paraffin embedded tissue. Methods of extracting nucleic acid from paraffin embedded tissue are well known in the art e.g., paraffin blocks containing the tissue are collected, de-waxed by treatment with xylene, treated with proteinase to remove protein contaminants, and then finally extracted with phenol and chloroform, followed by ethanol precipitation. Alternatively, nucleic acid from a paraffin embedded tissue can be isolated by commercially available kits e.g., EZ1 DNA kit, QIAamp DNA Mini Kit from Qiagen; Paraffin Block RNA Isolation Kit, RecoverAll™Total Nucleic Acid Isolation Kit from Ambion.

Nucleic acid need not be extracted, but may be made available by suitable treatment of cells or tissue such as described in U.S. patent application Ser. No. 11/566,169, which is incorporated herein by reference.

Polypeptides detected in the methods of the present invention may be detected directly from a biological sample or may be further purified for detection. Any known method for polypeptide purification may be used including, but not limited to, sucrose gradient purification, size exclusion chromatography, ion exchange chromatography, affinity chromatography, immunoaffinity chromatography, HPLC, gel electrophoresis (e.g. SDS-PAGE or QPNC-PAGE), and immunoprecipitation with a BCR-ABL1 specific antibody.

Detection

The BCR-ABL1 polynucleotides or polypeptides may be detected by a variety of methods known in the art. Non-limiting examples of detection methods are described below. The detection assays in the methods of the present invention may include purified or isolated DNA, RNA or protein or the detection step may be performed directly from a biological sample without the need for further DNA, RNA or protein purification/isolation.

Nucleic Acid Amplification—

BCR-ABL1 polynulceotides can be detected by the use of nucleic acid amplification techniques which are well known in the art. The starting material may be genomic DNA, cDNA, RNA mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include the polymerase chain reaction (PCR), reverse transcriptase polymerase chain reaction (RT-PCR), nested PCR, ligase chain reaction (see Abravaya, K., et al., Nucleic Acids Res. (1995), 23:675-682), branched DNA signal amplification (see Urdea, M. S., et al., AIDS (1993), 7(suppl 2):S11-S14, amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, or other sequence replication assays or signal amplification assays.

Primers:

Oligonucleotide primers for use amplification methods can be designed according to general guidance well known in the art as described herein, as well as with specific requirements as described herein for each step of the particular methods described.

In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length. There is no standard length for optimal hybridization or polymerase chain reaction amplification.

Methods of designing primers have been described in U.S. patent application Ser. No. 10/921,482. Primers useful in the methods described herein are also designed to have a particular melting temperature (Tₘ) by the method of melting temperature estimation. Commercial programs, including Oligo™, Primer Design and programs available on the internet, including Primer3 and Oligo Calculator can be used to calculate a Tₘ of a polynucleotide sequence useful according to the invention.

Tₘ of a polynucleotide affects its hybridization to another polynucleotide (e.g., the annealing of an oligonucleotide primer to a template polynucleotide). In the subject methods, it is preferred that the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In preferred embodiments, 100% complementarity is preferred.

Probes:

Probes are capable of hybridizing to at least a portion of the nucleic acid of interest or a reference nucleic acid. Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, about 100 nucleotides long.

However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe. For example, a detectable label includes a labeled antibody or a primary antibody/secondary antibody pair, wherein the detectable label may be in the primary antibody, or in the secondary antibody or in both.

Primers or probes may be prepared that hybridize under stringent conditions to the insert sequence or to a junction sequence that includes some normal BCR/Abl mRNA sequence and some of the adjoining insertion sequence. Such primers or probes can be designed so that they hybridize under stringent conditions to the specific splice variant transcript but not to normal BCR/Abl transcript. Primers or probes also can be prepared that are complementary and specific for the normal BCR/Abl splice junction. Such primers or probes can be used to detect the normal BCR/Abl mRNA and not the corresponding insertion mutation such as is described herein.

Detectable Label

The term “detectable label” as used herein refers to a molecule or a compound or a group of molecules or a group of compounds associated with an oligonucleotide (e.g., a probe or primer) and is used to identify the probe hybridized to a genomic nucleic acid or reference nucleic acid.

Detectable labels include but are not limited to fluorophores, isotopes (e.g., ³²P, ³³P, ³⁵S, ³H, ¹⁴C, ¹²⁵I, ¹³¹I), electron-dense reagents (e.g., gold, silver), nano articles enzymes commonly used in an ELISA (e.g., horseradish peroxidase, beta-galactosidase, luciferase, alkaline phosphatase), chemiluminiscent compound, colorimetric labels (e.g., colloidal gold), magnetic labels (e.g., Dynabeads™), biotin, digoxigenin, haptens, proteins for which antisera or monoclonal antibodies are available, ligands, hormones, oligonucleotides capable of forming a complex with the corresponding oligonucleotide complement.

One general method for real time PCR uses fluorescent probes such as the TaqMan® probes, molecular beacons, and Scorpions. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion™ and TaqMan® technologies are based on the principle of fluorescence quenching and involve a donor fluorophore and a quenching moiety.

TaqMan® probes (Heid, et al., Genome Res 6: 986-994, 1996) use the fluorogenic 5′ exonuclease activity of Taq polymerase to measure the amount of target sequences in cDNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo) benzoic acid (DABCYL). See Tyagi, et al., 16 Nature Biotechnology 49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase (e.g., reverse transcriptase) replicates a template on which a TaqMan® probe is bound, its 5′ exonuclease activity cleaves the probe. This ends the activity of the quencher (no FRET) and the donor fluorophore starts to emit fluorescence which increases in each cycle proportional to the rate of probe cleavage. Accumulation of PCR product is detected by monitoring the increase in fluorescence of the reporter dye (note that primers are not labeled). If the quencher is an acceptor fluorophore, then accumulation of PCR product can be detected by monitoring the decrease in fluorescence of the acceptor fluorophore.

In a preferred embodiment, the detectable label is a fluorophore. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Förster or fluorescence resonance energy transfer (“FRET”). Quenching by FRET is generally used in TaqMan® probes while proximal quenching is used in molecular beacon and Scorpion™ type probes.

Suitable fluorescent moieties include but are not limited to the following fluorophores working individually or in combination: 4-acetamido-4′-isothiocyanatostilbene-2,2′ disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120),7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC).

Detection of Nucleic Acid by Size:

Methods for detecting the presence or amount of polynucleotides are well known in the art and any of them can be used in the methods described herein so long as they are capable of separating individual polynucleotides by a difference in size. The separation technique used should permit resolution of nucleic acid as long as they differ from one another by at least one nucleotide or more. The separation can be performed under denaturing or under non-denaturing or native conditions—i.e., separation can be performed on single- or double-stranded nucleic acids. Useful methods for the separation and analysis of polynucleotides include, but are not limited to, electrophoresis (e.g., agarose gel electrophoresis, capillary electrophoresis (CE)), chromatography (HPLC), and mass spectrometry.

In one embodiment, CE is a preferred separation means because it provides exceptional separation of the polynucleotides in the range of at least 10-1,000 base pairs with a resolution of a single base pair. CE can be performed by methods well known in the art, for example, as disclosed in U.S. Pat. Nos. 6,217,731; 6,001,230; and 5,963,456, which are incorporated herein by reference. High-throughput CE apparatuses are available commercially, for example, the HTS9610 High throughput analysis system and SCE 9610 fully automated 96-capillary electrophoresis genetic analysis system from Spectrumedix Corporation (State College, Pa.); P/ACE 5000 series and CEQ series from Beckman Instruments Inc (Fullerton, Calif.); and ABI PRISM 3100 genetic analyzer (Applied Biosystems, Foster City, Calif.). Near the end of the CE column, in these devices the amplified DNA fragments pass a fluorescent detector which measures signals of fluorescent labels. These apparatuses provide automated high throughput for the detection of fluorescence-labeled PCR products.

In some embodiments, nucleic acid may be analyzed and detected by size using agarose gel electrophoresis. Methods of performing agarose gel electrophoresis are well known in the art. See Sambrook et al., Molecular Cloning: A Laboratory Manual (2nd Ed.) (1989), Cold Spring Harbor Press, N.Y.

DNA Sequencing:

In some embodiments, detection of nucleic acid is by DNA sequencing. Sequencing may be carried out by the dideoxy chain termination method of Sanger et al. (PNAS USA (1977), 74, 5463-5467) with modifications by Zimmermann et al. (Nucleic Acids Res. (1990), 18:1067). Sequencing by dideoxy chain termination method can be performed using Thermo Sequenase (Amersham Pharmacia, Piscataway, N.J.), Sequenase reagents from US Biochemicals or Sequatherm sequencing kit (Epicenter Technologies, Madison, Wis.). Sequencing may also be carried out by the “RR dRhodamine Terminator Cycle Sequencing Kit” from PE Applied Biosystems (product no. 403044, Weiterstadt, Germany), Taq DyeDeoxy™ Terminator Cycle Sequencing kit (Perkin-Elmer/Applied Biosystems) using an Applied Biosystems Model 373A DNA or in the presence of dye terminators CEQ™ Dye Terminator Cycle Sequencing Kit, (Beckman 608000). Alternatively, sequencing can be performed by a method known as Pyrosequencing (Pyrosequencing, Westborough, Mass.). Detailed protocols for Pyrosequencing can be found in: Alderborn et al., Genome Res. (2000), 10:1249-1265.

Detection of Polypeptide by Size:

Methods for detecting the presence or amount of polypeptides are well known in the art and any of them can be used in the methods described herein so long as they are capable of separating polypeptides by a difference in size. The separation can be performed under denaturing or under non-denaturing or native conditions. Useful methods for the separation and analysis of polypeptides include, but are not limited to, electrophoresis (e.g., SDS-PAGE electrophoresis, capillary electrophoresis (CE)), immunoblot analysis, size exclusion chromatography, chromatography (HPLC), and mass spectrometry.

Antibody Production and Screening

Various procedures known in the art may be used for the production of antibodies which bind to variants of the BCR-ABL1 protein. Such antibodies include but are not limited to polyclonal, monoclonal, chimeric, single chain, Fab fragments and fragments produced by a Fab expression library. Antibodies that specifically bind to an epitope of SEQ ID NOs: 10-12 are useful for detection and diagnostic purposes.

Antibodies that bind to the polypeptide of SEQ ID NOs 10-12 may also specifically detect and distinguish insertion/truncation variants of the BCR-ABL1 protein from other BCR-ABL1 proteins without such insertion/truncation mutations.

For the production of antibodies, various host animals, including but not limited to rabbits, mice, rats, etc, may be immunized by injection with the full length or fragment of variants of the BCR-ABL protein. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to BCR-ABL1 protein variants may be prepared by using any technique which provides for the production of antibody molecules by continuous cell lines in culture. These include but are not limited to the hybridoma technique originally described by Kohler and Milstein, (Nature (1975), 256:495-497), the human B-cell hybridoma technique (Kosbor et al., Immunology Today (1983), 4:72; Cote et al. Proc. Natl. Acad. Sci. (1983), 80:2026-2030) and the EBV-hybridoma technique (Cole et al., Monoclonal Antibodies and Cancer Therapy (1985), Alan R. Liss, Inc., pp. 77-96). In addition, techniques developed for the production of “chimeric antibodies” (Morrison et al., Proc. Natl. Acad. Sci. USA (1984), 81:6851-6855; Neuberger et al., Nature (1984), 312:604-608; Takeda et al., Nature (1985), 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce specific single chain antibodies which bind to variants of the BCR-ABL protein.

Antibody fragments which recognize variants of the BCR-ABL1 protein may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., Science. 1989; 246: 1275-1281) to allow rapid and easy identification of monoclonal Fab fragments with the desired specificity to variants of the BCR-ABL1 protein.

Antibodies to BCR-ABL1 variants can be used in a variety of techniques for detecting BCR-ABL1 polypeptides in the methods of the present invention including, but non limited to, immunoprecipitation, enzyme-linked immunosorbent assay (ELISA), protein immunoblotting techniques such as westerns, etc.

Cloning

The nucleic acid (e.g., cDNA or genomic DNA) encoding at least a portion of bcr-abl or its variants may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art, see Sambrook, et al., Molecular Cloning: A Laboratory Manual (1989), Second Edition, Cold Spring Harbor Press, Plainview, N.Y.

Prokaryotic Vectors:

Prokaryotic transformation vectors are well-known in the art and include pBlueskript and phage Lambda ZAP vectors (Stratagene, La Jolla, Calif.), and the like. Other suitable vectors and promoters are disclosed in detail in U.S. Pat. No. 4,798,885, issued Jan. 17, 1989, the disclosure of which is incorporated herein by reference in its entirety.

Other suitable vectors for transformation of E. coli cells include the pET expression vectors (Novagen, see U.S. Pat. No. 4,952,496), e.g., pET11a, which contains the T7 promoter, T7 terminator, the inducible E. coli lac operator, and the lac repressor gene; and pET 12a-c, which contain the T7 promoter, T7 terminator, and the E. coli ompT secretion signal. Another suitable vector is the pIN-IIIompA2 (see Duffaud et al., Meth. in Enzymology, 153:492-507, 1987), which contains the lpp promoter, the lacUV5 promoter operator, the ompA secretion signal, and the lac repressor gene.

Eukaryotic Vectors:

Exemplary, eukaryotic transformation vectors, include the cloned bovine papilloma virus genome, the cloned genomes of the murine retroviruses, and eukaryotic cassettes, such as the pSV-2 gpt system [described by Mulligan and Berg, Nature Vol. 277:108-114 (1979)] the Okayama-Berg cloning system [Mol. Cell. Biol. Vol. 2:161-170 (1982)], and the expression cloning vector described by Genetics Institute (Science. 1985; 228: 810-815), pCMV Sport, pcDNA™ 3.3 TOPO®, BaculoDirect™ Baculovirus Expression System (Invitrogen Corp., Carlsbad, Calif., USA), StrataClone™ (Stratagene, CA, USA), pBAC vectors (EMD Chemicals Inc, NJ, USA).

Vector Components:

Vector components generally include, but are not limited to, one or more of a regulatory elements such as an enhancer element, a promoter, and a transcription termination sequence, an origin of replication, one or more selection marker genes, and a cloning site.

Origin of Replication:

Both expression and cloning vectors contain a nucleic acid sequence that enables the vector to replicate in one or more selected host cells. Such sequences are well known for a variety of bacteria, yeast, and viruses. Non-limiting examples include the origin of replication from the plasmid pBR322 for most Gram-negative bacteria, the 2 plasmid origin is suitable for yeast, and various viral origins (SV40, cytomegalovirus, polyoma, adenovirus, VSV or BPV) useful for cloning vectors in mammalian cells.

Selection Marker:

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells is those that enable the identification of cells competent to take up the bcr-abl-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA (1980), 77:4216. A suitable selection gene for use in yeast is the trp 1 gene present in the yeast plasmid YRp7 (Stinchcomb et al., Nature (1979), 282:39-43; Kingsman et al., Gene (1979), 7:141-152). The trp 1 gene provides a selection marker for a mutant strain of yeast lacking the ability to grow in tryptophan, for example, ATCC No. 44076 or PEP4-1 (Jones, Genetics (1977), 86:85-102).

Regulatory Elements:

Expression and cloning vectors usually contain a promoter and/or enhancer operably linked to the bcr-abl encoding nucleic acid sequence to direct mRNA synthesis. Promoters recognized by a variety of potential host cells are well known. Promoters suitable for use with prokaryotic hosts include the beta-lactamase and lactose promoter systems (Chang et al., Nature (1978), 275:617-624; Goeddel et al., Nature (1979), 281:544-548), alkaline phosphatase, a tryptophan (trp) promoter system (EP 36,776), T7 promoter, and hybrid promoters such as the tac promoter (deBoer et al., Proc. Natl. Acad. Sci. USA (1983), 80:21-25). Promoters for use in bacterial systems also will contain a Shine-Dalgarno (S.D.) sequence operably linked to the DNA encoding bcr-abl.

Examples of suitable promoting sequences for use with yeast hosts include the promoters for 3-phosphoglycerate kinase (Hitzeman et al., J. Biol. Chem. (1980), 255:12073-12080) or other glycolytic enzymes (Holland and Holland, Biochemistry (1978), 17:4900-4907), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

bcr-abl transcription from vectors in eukaryotic host cells may be controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the bcr-abl by higher eukaryotes may be increased by inserting an enhancer sequence into the vector. Enhancers are cis-acting elements of DNA, usually about from 10 to 300 bp that act on a promoter to increase its transcription. Many enhancer sequences are now known from mammalian genes (globin, elastase, albumin, .alpha.-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the bcr-abl coding sequence, but is preferably located at a site 5′ from the promoter.

Vectors encoding bcr-abl nucleic acid sequence and its variants may further comprise non-bcr-abl nucleic acid sequence which may be co-expressed with bcr-abl and its variants either as a fusion product or as a co-transcript. Non limiting examples of such non-bcr-abl nucleic acid sequence includes His-tag (a stretch of poly histidines), FLAG-tag, and Green Fluorescent Protein (GFP). His-tag and FLAG-tag can be used to in many different methods, such as purification of BCR-ABL protein and or insertion/truncation mutant of BCR-ABL protein fused to such tags. The tags can also serve as an important site for antibody recognition. This is particularly important in detecting BCR-ABL proteins and or insertion/truncation mutant of BCR-ABL protein fused to such tags. GFP may be used as a reporter of expression (Phillips G. J. FEMS Microbiol. Lett. 2001; 204 (1): 9-18), such as the expression of bcr-abl and the splice variant of bcr-abl.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNA or cDNA. These regions contain nucleotide segments transcribed as polyadenylated fragments in the untranslated portion of the mRNA encoding bcr-abl.

Still other methods, vectors, and host cells suitable for adaptation to the synthesis of bcr-abl in recombinant vertebrate cell culture are described in Gething et al., Nature (1981), 293:620-625; Mantei et al., Nature. 1979; 281:40-46; EP 117,060; and EP 117,058.

Genetically Modifying Host Cells by Introducing Recombinant Nucleic Acid

The recombinant nucleic acid (e.g., cDNA or genomic DNA) encoding at least a portion of bcr-abl or its variants may be introduced into host cells thereby genetically modifying the host cell. Host cells may be used for cloning and/or for expression of the recombinant nucleic acid. Host cells can be prokaryotic, for example bacteria. Host cell can be also be eukaryotic which includes but not limited to yeast, fungal cell, insect cell, plant cell and animal cell. In preferred embodiment, the host cell can be a mammalian cell. In another preferred embodiment host cell can be human cell. In one preferred embodiment, the eukaryotic host cell may be K562 cell. K562 cells were the first human immortalized myelogenous leukemia line to be established and are a bcr-abl positive erythroleukemia line derived from a CML patient in blast crisis (Lozzio & Lozzio, Blood. 1975; 45(3): 321-334; Drexler, H. G. The Leukemia-Lymphoma Cell Line Factsbook. (2000), Academic Press.

Host cells may comprise wild-type genetic information. The genetic information of the host cells may be altered on purpose to allow it to be a permissive host for the recombinant DNA. Examples of such alterations include mutations, partial or total deletion of certain genes, or introduction of non-host nucleic acid into host cell. Host cells may also comprise mutations which are not introduced on purpose.

Several methods are known in the art to introduce recombinant DNA in bacterial cells that include but are not limited to transformation, transduction, and electroporation, see Sambrook, et al., Molecular Cloning: A Laboratory Manual (1989), Second Edition, Cold Spring Harbor Press, Plainview, N.Y. Non limiting examples of commercial kits and bacterial host cells for transformation include NovaBlue Singles™ (EMD Chemicals Inc, NJ, USA), Max Efficiency® DH5™, One Shot® BL21 (DE3) E. coli cells, One Shot® BL21 (DE3) pLys E. coli cells (Invitrogen Corp., Carlsbad, Calif., USA), XL1-Blue competent cells (Stratagene, CA, USA). Non limiting examples of commercial kits and bacterial host cells for electroporation include Zappers™ electrocompetent cells (EMD Chemicals Inc, NJ, USA), XL1-Blue Electroporation-competent cells (Stratagene, CA, USA), ElectroMAX™ A. tumefaciens LBA4404 Cells (Invitrogen Corp., Carlsbad, Calif., USA).

Several methods are known in the art to introduce recombinant nucleic acid in eukaryotic cells. Exemplary methods include transfection, electroporation, liposome mediated delivery of nucleic acid, microinjection into to the host cell, see Sambrook, et al., Molecular Cloning: A Laboratory Manual (1989), Second Edition, Cold Spring Harbor Press, Plainview, N.Y. Non limiting examples of commercial kits and reagents for transfection of recombinant nucleic acid to eukaryotic cell include Lipofectamine™ 2000, Optifect™ Reagent, Calcium Phosphate Transfection Kit (Invitrogen Corp., Carlsbad, Calif., USA), GeneJammer® Transfection Reagent, LipoTAXI® Trasfection Reagent (Stratagene, CA, USA). Alternatively, recombinant nucleic acid may be introduced into insect cells (e.g. sf9, sf21, High Five™) by using baculo viral vectors.

In one preferred embodiment, an exemplary vector comprising the cDNA sequence of bcr-abl splice variant (pCMV/GFP/79INS bcr-abl) may be transfected into K562 cells. Stable transfected K 562 cells may be developed by transfecting the cells with varying amounts of the pCMV/GFP/79INS bcr-abl vector (0 ng-500 ng) using various methods known in the art. In one exemplary method, The ProFection® Mammalian Transfection System—Calcium Phosphate (Promega Corporation, WI, USA) may be used. This is a simple system containing two buffers: CaCl₂ and HEPES-buffered saline. A precipitate containing calcium phosphate and DNA is formed by slowly mixing a HEPES-buffered phosphate solution with a solution containing calcium chloride and DNA. These DNA precipitates are then distributed onto eukaryotic cells and enter the cells through an endocytic-type mechanism. This transfection method has been successfully used by others (Hay et al. J. Biol. Chem. 2004; 279: 1650-58). The transfected K562 cells can be selected from the non-transfected cells by using the antibiotics Neomycin and Ampicillin. Expression of the spliced variant of bcr-abl can assessed from the co-expression of the reporter gene GFP.

Alternatively, in a 24-well format complexes are prepared using a DNA (μg) to Lipofectamine™ 2000 (Invitrogen Corporation, Carlsbad, Calif., USA) (μl) ratio of 1:2 to 1:3. Cells are transfected at high cell density for high efficiency, high expression levels, and to minimize cytotoxicity. Prior to preparing complexes, 4-8×10⁵ cells are plated in 500 μl of growth medium without antibiotics. For each transfection sample, complexes are prepared as follows: a. DNA is diluted in 50 μl of Opti-MEM® I Reduced Serum Medium without serum (Invitrogen Corporation, Carlsbad, Calif., USA) or other medium without serum and mixed gently. b. Lipofectamine™ 2000 is mixed gently before use and the mixture is diluted to appropriate amount in 50 μl of Opti-MEM® I Medium. The mixture is incubated for 5 minutes at room temperature. c. After 5 minute incubation, the diluted DNA is combined with diluted Lipofectamine™ 2000 (total volume=100 μl) and is mixed gently. The mixture is incubated for 20 minutes at room temperature. 100 μl of complexes is added to each well containing cells and medium. The contents are mixed gently by rocking the plate back and forth. Cells are incubated at 37° C. in a CO2 incubator for 18-48 hours prior to testing for transgene expression. Medium may be changed after 4-6 hours. Cells are passaged at a 1:10 (or higher dilution) into fresh growth medium 24 hours after transfection. Selective medium (containing Neomycin and Ampicillin) is added the following day.

Prediction of the Likelihood of Drug Resistance in CML Patients or Subjects Suspected of Having CML

Methods of the invention can be used for predicting the likelihood that a CML patient or a subject suspected of having CML with a BCR/ABL1 translocation will be resistant to treatment with one or more BCR-ABL1 kinase inhibitors. A sample from a CML patient, or a subject suspected of having CML, is assessed for the presence or absence of a polynucleotide sequence encoding the 79INS, 84INS, and/or 231INS BCR/ABL1 splice variants described herein, or a complement thereof. Optionally, a sample from a CML patient, or a subject suspected of having CML, is assessed for the presence or absence of a BCR-ABL1 fusion protein with a C-terminus having an amino acid sequence of any one of SEQ ID NOs: 10-12. Methods for detecting the absence or presence of such polypeptides are discussed above. The presence of the polypeptide sequence indicates that the patient has an increased likelihood of being resistant to treatment with one or more BCR-ABL1 kinase inhibitors relative to a patient not having the polynucleotide sequence. The presence of one or more of these BCR-ABL1 fusion proteins indicates that the patient has an increased likelihood of being resistant to treatment with one or more BCR-ABL1 kinase inhibitors relative to a patient not having the polynucleotide sequence.

In another embodiment, a sample from a CML patient, or a subject suspected of having CML, is assessed for the presence or absence of a polypeptide having an amino acid sequence of SEQ ID NO: 2, 4, or 6.

Identifying a Compound for Treating Leukemic Patients

In one preferred embodiment, cell lines expressing BCR-ABL (both wild-type and/or mutant) proteins may be utilized to screen compounds for treating CML patients. In preferred embodiments, the compounds may be targeting BCR-ABL protein. In some embodiments, the compounds may be inhibitor of ABL kinase activity. Non-limiting examples of kinase inhibitors include but not limited to imatinib, dasatinib, nilotinib, Bosutinib (SKI-606) and Aurora kinase inhibitor VX-680. In other embodiments, the compounds may not be an inhibitor of ABL kinase activity.

The effect of the compounds on the cells may be assessed. Several parameters may be assessed for identifying the compounds that may be beneficial for treatment of CML patients. Non-limiting examples of the parameters that may be assessed includes cell viability, cell proliferation, apoptosis, kinase activity of BCR-ABL protein, additional mutations in BCR-ABL protein, additional mutation in ABL protein.

In one embodiment, human chronic myeloid leukemia (CML) cell lines expressing BCR-ABL (both wild-type and/or mutant) proteins may be used to study the effect of such compounds on their effect on the cells. Non-limiting examples of human chronic myeloid leukemia (CML) cell lines include BV173, K562, KCL-22, and KYO-1, LAMA84, EM2, EM3, BV173, AR230, and KU812 (Mahon, F. X., Blood. 2000; 96: 1070-1079; Lerma et al. Mol. Cancer Ther. 2007; 6(2): 655-66).

In other embodiments, non-CML cells may be transfected with expression vectors comprising bcr-abl gene or variants of bcr-abl gene including splice variants of bcr-abl gene resulting in genetically modified cells comprising the recombinant polynucleotide. Thus, the transfected cells will be able to express BCR-ABL protein or its variants. The genetically modified cells can be used to screen compounds for treating CML patients.

In yet other embodiments, CML cell lines, for example BV173, K562, KCL-22, and KYO-1, LAMA84, EM2, EM3, BV173, AR230, and KU812 may be transfected with expression vectors comprising splice variants of bcr-abl gene resulting in genetically modified cells comprising the recombinant polynucleotide. The gene product of the splice variants of bcr-abl gene, the insertion/truncation mutant of BCR-ABL may impart partial or total resistance to ABL kinase inhibitors to these genetically modified cells. The genetically modified cells may be used to screen compounds for treating CML. The compounds may be inhibitors of ABL kinase activity or these compounds may have other mechanism of action.

The CML cell lines and the genetically modified cell lines as discussed above may be grown in appropriate growth medium and using appropriate selective antibiotics. Methods for cell culture is well known in the art (Sambrook, et al., Molecular Cloning: A Laboratory Manual (1989), Second Edition, Cold Spring Harbor Press, Plainview, N.Y.). Several growth media for cell culture are commercially available. Non-limiting examples include GIBCO® RPMI Media 1640, Dulbecco's Modified Eagle Medium (DMEM), DMEM: Nutrient Mixture F-12 (DMEM/F12), Minimum Essential Media (Invitrogen Corp., Carlsbad, Calif., USA), RF-10 medium. Non-limiting examples of selective antibiotics include ampicillin, neomycin, Geneticin®, Hygromycin B.

In one preferred embodiment, K562 cells (ATCC catalog no: CCL-243) may be genetically modified by transfecting with different amounts of the expression vector pCMV/GFP/35INS bcr-abl. In one embodiment, the amount the expression vector pCMV/GFP/35INS bcr-abl used for transfection can be 0 ng, or can be at least about: 1 ng, 2 ng, 5 ng, 7.5 ng, 10 ng, 12.5 ng, 15 ng, 20 ng, 25 ng, 30 ng, 40 ng, 50 ng, 60 ng, 75 ng, 100 ng, 125 ng, 200 ng, 500 ng, 750 ng, or 1 μg. The transefected cells may be grown in RF-10 medium with neomycin/and or amplicillin.

Assessing the Effect of a Compound for Treatment of Leukemia on Genetically Modified Cells

Several parameters may be assessed for identifying the compounds that may be beneficial for treatment of CML patients. Non-limiting examples of the parameters that may be assessed includes cell viability, cell proliferation, apoptosis, kinase activity of BCR-ABL protein, additional mutations in BCR-ABL protein, additional mutation in ABL protein.

Cell Viability:

Cells can be plated at a density of 2-2.5×10⁵ cells/mL in RF-10 with varying amounts of the compound or without the compound. Aliquots are taken out at 24-hour intervals for assessment of cell viability by tryphan blue exclusion.

Alternatively, cell viability can be measured by colorimetric assay such as MTT assay (Mosman et al. J. Immunol. Meth. 1983; 65: 55-63). Commercial kits for MTT assay are available. For example, CellTiter 96® Non-Radioactive Cell Proliferation Assay (MTT) (Promega Corporation, WI, USA), Vybrant® MTT Cell Proliferation Assay Kit (Invitrogen Corp., Carlsbad, Calif., USA).

Cell Proliferation:

Proliferation of the genetically modified cells in presence of a compound for treatment of CML patient can be measured in several ways. The proliferation of the cells can be indicative of the effectiveness of the compound for CML therapy.

In one such method, cell proliferation assay can performed using MTS tetrazolium such as Cell Titer96 Aqueous(Promega corporation, WI, USA), which measures numbers of viable cells. Between 2×10³ and 2×10⁴ cells are washed twice in RF-10 and plated in quadruplicate into microtiter-plate wells in 100 μA RF-10 plus various doses of the compound. Controls using the same concentrations of compound without cells are set up in parallel. Twenty microliters MTS is added to the wells at daily intervals. Two hours after MTS is added, the plates are read in a microplate auto reader (Dynex Technologies, Billingshurst, UK) at 490-nm wavelength. Results are expressed as the mean optical density for each dose of the compound. All experiments are repeated at least 3 times.

In another method, cell proliferation assay can be performed by monitoring the incorporation bromo-deoxyuracil (BrdU) into newly synthesized DNA. Amount of BrdU incorporated into the DNA will be proportional to the amount of DNA synthesis and will be indicative of the proliferating cells. In one such method, detectably labeled anti-BrdU antibody can be used to measure the amount of BrdU incorporated into the cells treated with various amounts of the compound. In one embodiment, the detectable label can be FITC. The amount of signal from FITC-labeled anti-BrdU bound to the DNA can be measured by Flow Cytometry. Commercially available kits for flow cytometry based cell proliferation assays are available. Such as, Click-iT® EdU (Invitrogen Corp., Carlsbad, Calif., USA). ELISA based assays for measuring BrdU incorporation by proliferating cells care commercially available examples include BrdU Cell Proliferation Assay kit (Calbiochem, EMD Chemicals Inc, NJ, USA).

In another method, proliferation of cells treated with various amounts of the compound can be measured by monitoring the incorporation of radioactively labeled deoxynucleotides (Sun et al. Cancer Res. 1999; 59: 940-946).

Kinase Activity of BCR-ABL:

The effect of a compound on the kinase activity of the BCR-ABL is assessed by monitoring tyrosine phosphorylation profile of the cellular proteins. CrlkL is a substrate of BCR-ABL tyrosine kinase (Ren et al. Genes Dev. 1994; 8(7): 783-95). Genetically modified cells comprising recombinant bcr-abl or variant so of bcr-abl including the splice variant are grown in presence of various amounts of a compound for treating CML patients. In a preferred embodiment, the compounds are ABL tyrosine kinase inhibitors. Non-limiting examples of kinase inhibitors include imatinib, nilotinib, dasatinib, Bosutinib (SKI-606) and Aurora kinase inhibitor VX-680. Amount of phosphorylated CrkL protein can be measured by using detectably labeled anti-phospho CrkL antibody. In one embodiment, the detectable label is phycoerythrin. The signal can be detected by Flow cytometer. Alternatively, the signal can be detected by Fluorescent Microtiter plate reader.

Sequencing of the ABL Kinase Domain:

To further investigate the reason for some cells that do not overexpress BCR-ABL but that have higher resistance to a compound that target the ATP-binding site of the ABL kinase domain (such as imatinib, nilotinib, dasatinib, and Aurora kinase inhibitor VX-680) than their sensitive counterparts, the entire kinase domain of K562-sensitive and -resistant cells can be sequenced. Sequencing can be performed using ABI prism 377 automated DNA sequencer (PE Applied Biosystems; USA). Sequence analysis can performed using the GCG version 10 software. The invention will now be described in greater detail by reference to the following non-limiting examples.

### Example 1

### Sample Collection

Peripheral blood samples were collected from 210 patients having either CML (n=204) or Philadelphia chromosome-positive acute lymphoblastic leukemia (ALL) (n=6) with resistance to more than one of the three kinase inhibitors: imatinib, nilotinib and dasatinib.

All samples were collected and analyzed according to a protocol approved by Institutional Review Board (IRB). Venous blood (5-8 ml) was collected from patients using BD Vacutainer™ CPT™ tubes (Beckton Dickenson, NJ, USA, Catalog number: 362760) by venous puncture. Peripheral mononuclear cells and platelets were isolated using manufacturer's protocol. Briefly, venous blood collected into the CPT™ tube was mixed with the anticoagulant present in the tube by inversion. The blood sample was centrifuged at 1500-1800 RCF for 20 min at room temperature (18° C.-25° C.). Plasma was removed from the top by aspiration without disturbing the white cell layer containing peripheral mononuclear cells and platelets. The peripheral mononuclear cells and platelets were carefully removed with a Pasteur pipette and collected in a separate tube.

RNA was extracted from blood cells using a MagNA Pure instrument (Roche, Indianapolis, Ind.). BCR-ABL1 fusion transcripts were amplified in the first round of RT-PCR, to ensure that normal ABL1 was not amplified. This step utilized a reverse primer annealed at the ABL1 exon 9-10 junction, along with 2 forward primers annealed at BCR exons b2 and e1. A semi-nested PCR was then performed to amplify the region encoding the abl1 kinase domain (exons 4-9), and the resulting products were purified and sequenced in both directions using the ABI Prism BigDye® Terminator v3.1 Cycle Sequencing Kit with detection by an ABI PRISM 3730 Capillary Genetic Analyzer (Applied Biosystems, Foster City, Calif., USA). Sequence data were then base-called, assembled, and analyzed with ABI Prism® SeqScape software (Applied Biosystems) using the c-ABL sequence (GenBank accession number U07563) as a reference. A mutation was considered to be present only if it was detected in both strands in 2 or more independent runs.

### Example 2

### bcr-abl1 Mutation Detection and Analysis

BCR-ABL1 fusion transcripts were amplified in the first round of RT-PCR using a reverse primer annealed at the ABL1 exons 9-10 junction, and two forward primers annealed at BCR exons b2 and e1. The multiplexed RT-PCR is designed to encsure that b2a2/b3a2 and e1a2, the most frequent BCR-ABL1 transcripts, are all amplified. A semi-nested PCT was then performed to amplified ABL1 kinase domain (exons 4-9).

The resulting products were purified and sequence in both directions using the ABI Prism BigDye® Terminator v3.1 Cycle Sequencing Kit with detection by an ABI PRISM 3100 Capillary Genetic Analyzer (Applied Biosystems, Foster City, Calif., USA). Sequence data were then base called, assembled and then analyzed with ABI Prism® SeqScape software (Applied Biosystems) using the c-ABL sequence (GenBank accession number U07563) as a reference. A mutation was considered to be present only if it was detected in both strands in two or more independent runs.

### Example 3

### bcr-abl1 79INS, 84INS and 231 INS Mutants

Three mutations, resulting from alternative splicing in the ABL1 kinase domain, were detected in 3 resistant CML patients. The key characteristics of these 3 mutations are summarized in Table 1 below.

TABLE 1
Characteristics of the 3 alternative splicing mutations detected
in multidrug resistant CML patients.

| | Nucleotide | Amino acid # at | Source of | Insertion | | |
|---|---|---|---|---|---|---|
| Splicing | # at | insertion (exons | inserted | size | Protein | Additional |
| mutation | insertion* | junction) | sequence+ | (nt) | change | mutations |
| 79INS | 1423-1424 | A474-C475 | Intron 8 | 79 | C475YfsX11 | None |
| | | (exons 8-9) | | | | |
| 84INS | 1270-1271 | W423-A424 | Intron 7 | 84 | A424EfsX18 | P309R homo |
| | | (exons 7-8) | | | | in exon 6 |
| 231INS | 822-823 | K274-E275 | Intron 4 | 231 | E275LfsX41 | Q300R het |
| | | (exons 4-5) | | | | in exon 5 |

*GenBank ID # M14752; +GenBank ID# U07563.

The 79INS variant carries an insertion of a 79-nt sequence derived from intron 8 into the ABL1 exons 8-9 junction at T1423/G1424 position. The inserted fragment comprises two separate sequences located 160-nt apart: the previously described 35-nt sequence (35INS) (Lee et al., Mol. Cancer Ther. 2008 7:3834-41, 2008; Ma et al., Acta Haematolology 2009 121:27-31), and an additional 44-nt segment downstream of 35INS. Nevertheless, the 79INS splicing mutant encodes the same protein as 35INS(C475YfsX11) which lacks the ABL1 C-terminus responsible for nuclear localization and DNA binding. This particular patient shows no additional mutations in the ABL1 kinase domain.

The 84INS splicing mutant contains an 84-nt sequence from intron 7 (approximately 1500-nt upstream of exon 8) inserted into the ABL1 exons 7-8 junction, causing a frameshift and protein truncation (A424EfsX18). In addition to the splicing mutation, there is a point mutation at nucleotide 926 (C to G; P309R) in the ABL1 exon 6.

The 231INS spliced transcript was first noticed by the detection of unusual large size of the PCR product (1100 bp+) after gel electrophoresis, while the regular BCR-ABL1 transcript which is significantly smaller in size (863 bp), was completely missing. 231INS has a 231-nt sequence from intron 4 (330-nt upstream of exon 5) integrated into the ABL1 exons 4-5 junction at G822/G823 position. This resulting insertion adds 40 intron-encoded amino acids, leading to a frameshift and early translational termination of BCR-ABL1 in the P-loop region (E275LfsX41). Based on the documented resistance to kinase inhibitors in this patient, this splicing mutation is likely responsible for the resistance. In addition to the splicing mutation, the transcript showed a point mutation in exon 5 at nucleotide 899 (A to G; Q300R) which is detected in less than 60% of the total BCR-ABL1 transcript.

The three identified mutants showed retention of intron sequences at the junction of two adjacent exons supporting aberrant alternative splicing as the cause for the insertions. In addition, the new pseudo-exons interrupt the open reading frame of the BCR-ABL1 transcript and cause frameshift leading to premature stop codons and various truncated BCR-ABL1 proteins.

Approximately 40-60% of human genes undergo alternative splicing, and alterations in alternative splicing have been manifested by its clinical connections to many human diseases, including cancers (Caceres & Kornblihtt, Trends in Genetics 2002 18:186-93; Stoilov et al., DNA and Cell Biol. 2002 21:803-18; Wu et al., “Alternatively Spliced Genes,” In Encyclopedia of Molecular and Cell Biology and Molecular Medicine, Vol. 1, 2^{nd} ed., 125-177 (2004)). Alternative splicing mutations in patients with CML being treated with tyrosine kinase inhibitors could be an overlooked mechanism for the resistance to therapy. Three alternative splicing mutations, 79INS, 84INS and 231INS, were detected in 3 multidrug resistant CML patients, in which they are the only isoform of BCR-ABL1 transcript to be detected. Additionally, these splicing abnormalities have not been detected in over 2000 tested samples from patients with CML being treated with imatinib, but showed no resistance (data not shown). Finally, more than 30 newly diagnosed CML patients were tested and none showed evidence of the expression of any of these alternatively spliced transcripts.

In summary, each of the three splicing variants described herein 1) are created by an exonic insertion of a sequence from the adjacent preceding intron; 2) produce an exclusively-expressed splicing variant in the absence of wild-type BCR-ABL1 transcript; 3) cause frameshift, early translational termination and truncation of the BCR-ABL1 protein missing a significant portion of the C-terminal regulatory region; 4) are associated with significant drug resistance not only to imatinib, but also to one or more of the newer tyrosine kinase inhibitors—nilotinib and dasatinib. These 3 identified mutations, along with the previously reported 35INS (Lee et al.,; Ma et al.,), appear to form a new class of alternative splicing mutations.

Unless otherwise defined, all technical and scientific terms used herein have the same meaning as commonly understood by one of ordinary skill in the art to which this invention belongs. All nucleotide sequences provided herein are presented in the 5′ to 3′ direction.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

1. A method for predicting likelihood for resistance of a patient with a Bcr/Abl translocation to treatment with one or more BCR/Abl kinase inhibitors, comprising:
assessing the bcr-abl mRNA in a sample obtained from the patient for the presence or absence of a polynucleotide sequence encoding the 79INS BCR/Abl splice variant, the 84INS BCR/Abl splice variant, or the 231 BCR/Abl splice variant; and
identifying the patient as having an increased likelihood of being resistant to treatment with one or more BCR/ABL kinase inhibitors when a polynucleotide encoding at least one of said splice variants is detected.

2. The method of claim 1, wherein the patient is diagnosed as having a myeloproliferative disease.

3. The method of claim 2, wherein the myeloproliferative disease is chronic myelogenous leukemia (CML).

4. The method of claim 1, wherein said one or more kinase inhibitors are selected from a group consisting of imatinib, nilotinib, bosutinib, and dasatinib.

5. The method of claim 4, wherein said one or more kinase inhibitors is imatinib.

6. The method of claim 1, wherein the sample comprises blood cells.

7. The method of claim 6, wherein the sample comprises peripheral mononuclear cells.

8. A vector comprising a recombinant polynucleotide, wherein said recombinant polynucleotide encodes the 79INS BCR-Abl splice variant, the 84INS splice variant, or the 231 BCR-Abl splice variant.

9. The vector of claim 8, wherein said polynucleotide is operably linked to an expression regulatory element, wherein said expression regulatory element is capable of modulating the expression of said recombinant polynucleotide.

10. The vector of claim 9, wherein said regulatory element comprises a promoter.

11. The vector of claim 9, wherein said regulatory element comprises an enhancer.

12. The vector of claim 9, wherein said regulatory element comprises a poly-adenylation signal.

13. The vector of claim 8, wherein said vector is an expression vector.

14. The vector of claim 8, wherein said vector is an eukaryotic expression vector.

15. The vector of claim 8, wherein said vector is a prokaryotic expression vector.

16. A vector of claim 8, wherein said vector is a viral vector.

17. A method for predicting likelihood for resistance of a patient with a Bcr/Abl translocation to treatment with one or more BCR-Abl kinase inhibitors, comprising:
assessing the BCR-Abl protein in a sample obtained from a patient for the presence or absence of a truncated Abl protein encoded the 84INS BCR/Abl splice variant or the 231INS BCR/Abl splice variant; and
identifying the patient as having an increased likelihood of being resistant to treatment with one or more BCR-ABL kinase inhibitors when at least one of said truncated proteins is detected.

18. The method of claim 17, wherein the presence or absence of said polypeptide is determined by assessing the size of said BCR-ABL protein.

19. The method of claim 17, wherein the presence or absence of said polypeptide is determined using an antibody that specifically binds to the BCR-ABL protein encoded by the 84INS BCR/Abl splice variant or the 231INS BCR/Abl splice variant.

20. The method of claim 17, wherein the patient is diagnosed as having a myeloproliferative disease.

21. The method of claim 20, wherein the myeloproliferative disease is chronic myelogenous leukemia (CML).

22. The method of claim 17, wherein said one or more kinase inhibitors are selected from a group consisting of imatinib, nilotinib, bosutinib, and dasatinib.

23. The method of claim 22, wherein said one or more kinase inhibitors is imatinib.

24. The method of claim 17, wherein the sample comprises blood cells.

25. The method of claim 17, wherein the sample comprises peripheral mononuclear cells.

26. The method of claim 17, wherein the C-terminus of the Abl protein encoded the 84INS BCR/Abl splice variant comprises the amino acid sequence of SEQ ID NO: 4

27. The method of claim 17, wherein the C-terminus of the Abl protein encoded the 231INS BCR/Abl splice variant comprises the amino acid sequence of SEQ ID NO: 5


SEQUENCE LISTING (15 sequences)

SEQ ID NO: 1 - protein, 242 aa, Homo sapiens
KTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGNEHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPFSAEIHQAFETMFQESSISDEVEKELGK

SEQ ID NO: 2 - protein, 214 aa, Artificial Sequence
  Description of Artificial Sequence Synthetic polypeptide
KTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGNEHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRAYFDNREERTR

SEQ ID NO: 3 - protein, 214 aa, Artificial Sequence; identical to SEQ ID NO: 2
  Description of Artificial Sequence Synthetic polypeptide

SEQ ID NO: 4 - protein, 170 aa, Artificial Sequence
  Description of Artificial Sequence Synthetic polypeptide
KTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGNEHLVKVADFGLSRLMTGDTYTAHAGAKFPIKWTAPESLAYNKFSIKSDVWETGFHCVSQDGLDLRTS

SEQ ID NO: 5 - protein, 44 aa, Artificial Sequence
  Description of Artificial Sequence Synthetic polypeptide
KTLKLSHTPTSSKRWGSTCGLPSRHQKQVVGTCKPALPCCLGEN

SEQ ID NO: 6 - DNA, 3840 bp, Homo sapiens
ggccttccccctgcgaggatcgccgttggcccgggttggctttggaaagcggcggtggctttgggccgggctcggcctcgggaacgccaggggcccctgggtgcggacgggcgcggccaggagggggttaaggcgcaggcggcggcggggcgggggcgggcctggcgggcgccctctccgggccctttgttaacaggcgcgtcccggccagcggagacgcggccgccctgggcgggcgcgggcggcgggcggcggtgagggcggcctgcggggcggcgcccgggggccgggccgagccgggcctgagccgggcccggaccgagctgggagaggggctccggcccgatcgttcgcttggcgcaaaatgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatccgctgagcagcgggatcaatggcagcttcttggtgcgtgagagtgagagcagtcctagccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccatgacagtggctgag

SEQ ID NO: 7 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 8 - DNA, 84539 bp, Homo sapiens
gatccgccctcctcagcctcccaaagtgctgagattacaagtcaccacgcctggcttccgtatttaagtattaggtggtgactccatacaaaatatttaacagcttgctggttaacatttaaatctttgttgctaagaccttttctatgtttgacagttattccaaacacgttccatataggaatagtttgccaaatagaagaacacgggatattcttctcccatggctgatgtgttgtgattctaggtgcatatttctgtttccaagaacacggacctgttctcaagttcaaacactgcatcaagagactgtgccccatgtcttttcacttttttccctctctctttctctccccttcccctccttgatttaatttcttcagcaggtcagaattgatacagcttccagaagaaatagtagaagtaggcaaaagattagtttttaattcttgttctgtcaaatactttttgattgaaattgtttttattacaaaggtagcatttgctttttgaaaaacaaaataagaaaaatctttttaatcacttcactattaagtttgttgaatattccctcataactttttctcctggctttttgagtatttgagacatttctttccacattgcagttttgaaacattctaccccacactcatacatgaaagtgaaggctctgcttctcagagtacagtttaatttactttgtggacatcattaacaaaaggcagagaaagacattggaccatactaattgtgggacttagaaaagctatattagtgatggttattaagtatcgcttgcagtggagtctgtaacatattcctatgaagttaatctaactactgagatagtgggagtgcagaggaggcatgctgtgagatggagtggtattttccagcatggcttttctgtttcttgaaaaccctacagcactttgccatggtttctcatctggatgaaataaagcacctgtaccttagtatgtcctgccatgcctttgtatggcatacttggctttgttctccaggcctcttcccacagtaacaacggattctttaggaattggaagggggggccgggcatggtgactcacgcctgtaatcctgggactttgggaggctgaggcgggtggatcacgaggtcaggagtttgagaccaacctggccaatatggtgaaaccctgtctctactaaaaatacaaaaaagaattagctgggtgtggtggggcatgcctgtagtcccagctactcgggaggctgaggcagaagaattgcttgaacccaggaggcggaggttgcagtgagctaagatcgtgccactgcactccagcctgggtggcagagactccgtctcaaaaaaaaaaaaaaaaaagaattggaagggaatggaggaagcaaggtggttaaacaaaaatcatctttacacacaccacccactctatgatgtatatgagttcagaagagcactttgctttatccttaatagaaatatctgtaaagccaggggagtcataaagctgtaggctaagtaaggtgccatgagaaaaggaggcttcacttacgccagtatttggagcagcgtttggcctcttggaggtgtttgcacccatcggagatgctccatagtgagatttaggataagtgcaaggtgagctttgcttttgtaaaatgcatgaagtgtgaccacgaaagggccatgggaaagaattattgatgggttacaggtgcatcgtttcaggcagattgacactaggaaagagcaaagagcaggtagaggtttgacagtttactcctttaaagtaatccatgtccaataacccatggaaagtcttcatgtacttctaaggggtagacataccctagtctaggtgctaatctaggttctatcatgtgttagctgtgaccctgggcaaataactttatcctatggcctcaatttcatcttgcaaaaaaatacggattagataatggcacttccttaaaaggtttgatgggatttcaatggaatattgcatagaaagtgcttgaggcattgtaagtacacagtaagtgaagtgttgaccattattaatatcatgatgataaagtgtgtaacttattttataagtaccatataattgaaatttgccaatagtaactcctcggagacttccagggaactctgtcaatcaaaaaaagctttgtgtaataatggacgctaccttgacagagtgtgttagtcaagagaggatacttatttataaattttgggatatttatacttttttttttttctttagagatggagtcttgctctgtcgccaaggctagagtgcagtggcgcaatctcggctcactgcaacctctgcctccccagttcaagtgattctcctgcctcagccttccaagtagctaggactacaggcacctgtcaccaggcctggctaatttttgtattttttaggagagacggggtttcaccatcttggtcaggctggtctcgaactcctgacctcgtgatccacacgcctcagccttccaaagtgctgggattacagacgtgagccaccgcacccaactataaaatgtatttttatagatacttattgaattttaggatgtgggctgggtgattattttttaaagtacttttaaaaaaattaaggtattatttacatataatatccacccatttttaagtatacattttggtgaattactgtgtgtcaatcatgtaacctcaaacacagccaagattataaaacagttctctacgctgctttggtgatggatacaccaaaatctcagaaaccaccactacagaacttattcatgtaaccaaacaccatgtgttccccaaaaacctattgaaaaaaaaaaagaacccaaaaccaaaaaacagttctctcaacattaaaagttttcctgtgcctttttccagtggattccttccctcagcctctggccccagacaaacacttatccactttctgtgtctgtagttttgtcttttcaagaaattcctatgaatggaaccatataaaagttatataaaaatcatagacttttgtatttgatttcttttactcttagcatcatttttttgagatttactcctgttgtatgtatttacatttcctctttttcttggtgagtaatattgcattatatgaatatctcacaatttatccattttactagtggataacattagattgcttccaacttgaggctattatagtacagctgctatgaacatttgtgtattcaccattgtgtgagcatatgattttccctaccttcttttctatttcactttcttttcctttttctttccttttttttttttcttttttttgagacagtctctctgtgtcgcccaggctggagtgcagaggcacgatctcggctcactgcaacctctgcctgctgggttcaagcaattctcagcgtcagcctcccaagtagctgggattacaggggcccgccaccacacccagctaatttttgtatttttagtagagacggggtttcaccctgttgaccaggctggtcttgaatgcctggcctcagtgacccacctacctcagcctcccaaagtgctgggattataggcctgagccacagtgcccggccctttttcctttctttttttgagataggatcttgctcaatcatgcaggctggagtgcagtggcacacttggctcactgcaacctccacctcctgggttcaagcgagtctcctgcctcagcctcccgagcagctgggactacaggtgcgtgccaccacgcccagttaatttctgtgtttttagtagagacggggtttcaccatgttggccaggatggtctcgatctcttgacctcgtgatctgcctgcttcagcctcccaaagtgctgggatgacaggcgtgagccaccaagcccagctgacaatggattttctttgttttctccttcatctgaagatgtcttccctttgccttcatttttaaaggatattttctctgggtatagaattataatccgagagtatagaggttgacaagttttttggtattggttttagttttgggttgtgtctttttgttgttggcttttgttttgtttttgccccttatagatgtttcactgtcttttggctcatgtgttccgatgagaagtcagcccttcattgggtccttgttctcatctgtttaatacctgttcccatacctgccctttctctctcactgcttttaagatttgctctttatctttggttttggagtatttgactatgaagtgcaagctgctttgtttcttcttggtgtacattcagcatcttgcatcttcaagtatctgcgtatcattaagtttgggcagtttttggctcttgtttcttctaaattttttctaccccattctccctgtccctctccttttggtattgtagttacccactggaacacgtgacattatgtagcaagtctctgagtctgttcatttttttccttcagtcttttttttttttttttgaaatggtctctctctgttgcccaggctggatggtacagcggtgtgatcacgactcactgcaactttggcctcctgggtttaagccatcctcctgcctcagcctctttgagtagctgggaccacaggcacgtgccaccacgcctggctaattttttgatttttactagaaacagagtcttgccatgttgcccaggctggtcttgaactcctgggctcaagcaatccacctgccctggtctcccaaaatgctaggattacaggtgtcagccactatgccagccttttcaattttttatctctcttcttcatcttggttaatttctactcatcttttttgaaatgcacatactgtccttttctttgtccattcagctgagtatatccagtgaattttttatttcaaaaagttaatttttttcagttctagaattgccatttaattattttttgttgttcctatttcccccattgagatttcctacttcttagctgagattttcattcattggaaacatgccttatactgcttcattgagatgattataatagctgcttaaaatctttgtctattccagcattttgttgatctgcaggttagactggcagttgaactaatttgtcccactgtcttgcttctttgtatggtgggtagttctggattgtttactggactgtatgaatgtcaaattctggatattctggattctgttacttttctctaatacttgtttggttttatcaggtggttttcttggctgggtttgaacacaaatgtgtttctctctgagatctttagtctgtagctgagctgctttgtgtctgtcccatgcatgagtggctcaagggtcagtcagggatgtgattgtacggggattggagatcttcccctgcctttttcccttctaggattacttcagtctatttatttatttgtttatttatttagagacagaagtcttcctctgtcacccaggctggagtgcaatggcgcgatctcagctcactgcaacccctacctcccgggttcaagcgattctcctgcctcagcctcctgagtagctgggattacaggcccgcaccaccacgcccggctaatttttgtattcttagtagagaaggggttttgccatgttggccaggctggtcacgaactcgtgaactcatgctctacccgcctcagcctccccaagtgctgggattacaagcgagagccactgcgcccagccggctctctttgttattcatagtttcctggcttctcttttctgagccacccagccaaaaatgctttgactttttctatgcatgtttctgtcatgtctgtgcatgacataaatcataatatcctaagacacaaaattgttaaaggcaataatttggaaacatacaacaataaataaaatggttatagagatgtaactttaaaaagttgaacattttgttaaaacgattcctgaatatgctggaaaattcagatagttaacaaaatattttgaggataaaaaggaaaggaatctgttatctggaggtagaataacttggccattttacagaaaggaaacaaattctgatttcactttttgtatatcatccatttgacaattttcaataaaaggtttattgaaaacggaatacataaggctggtgtttggacctttttcctaattagaaattgtccatgtctccaaagattattaactcagtttattatcagctaaagagatcttaaagttgactaaacacctggaaattaaaacttaggccaacacatcaaagtctttatgacttgaagtgttttaagaattaatgaggcaaagcacacacctgtggtcccagctacccaggagactgaggcaggagaactgtttgagcccaggagtttgaagctgcagtgagctatgatcatgccactgttcttcagcccaagagactgagtgagacctcttaagaaaaaaaaaaaaatgaccggacacagttgctcacgcctgtaatctcagcactttgggaggctgaggcgggtggatcacgagatcaggagatcgagacaatcctggctaacacggtgaaaccctgtctctactaaaaaaatacaaaaaaattagcagggcgtggtggtgggcgcctgtagtaccagctacctgggagcctgaggcagaaaaacggcgtgaacccaggaggcggatctcggcagtgagctcacaccagtggctcacacttgtaatcccagcactttgggaggccgaggtgggcagatcacctgaggtcaggagttcgagacaagcctgaccaacatggagaaaccccgtctctactaaaaatacaaaattagtgggcgtgtggcgcatgcctgtaatcccagctacttaggaggctgaggcaggagaatcgcttgaacccgggaggtggaggttgcagtgagccaggatcgcgccattgcactccagcctgggcaacaagagccaaactccgtctcgaaaagaaaaaattcatgagactaaatcattttaaaaagatgtcttttattacaatttctttcaattaaacagttttatatttttgaaattttaaacaatttgtgcaaacgagatcctactggacctataaaactaatgtagttaatttagaaggtttaaatcacgagaagttaagcccaaatcttgggttgatttaattgttgtgttaacattgtttttatatcataataaaatcagaaggatgcatagacttgtttttaagctaatattaacttctatttctagcccaaagagtcatctatcaaaagccagctcatattaagtagggggaccaagagggaggtctctcagttaaacaaagttattgacttgtctttagacaggcaagacagagtatctcctcataaggttttcttttaacctttttgttctgaagttaatatgtcttttaagtgaataatcttgtttataacaacattttcttttctgttttgtttgtttgtttgtttgagacagagtctcgcactgtcacccaggctggagtgcagtggcacagtcttggctcactgcaacctctgcctcctgggttcaagtgattctcctgcctcagcctcccgagtagctgggactacaggcacatgccaccactcccatctaatttgttttgtgtttttagtagagacggggtttcacaatattggtcaggctggtctcgaactcctgacctcgtgattcacccacctcggcctcccaaagtgctgggataacaggcatgagccactgcacccagctataacaacattttcaaagtggcacattttaatatgaataaaccttctacaatcacaggcctagaaagtttttctttttcttacggaatttcttgactgtttacttgtactagatatttcttcggtatattcataatgacagaacaattgtttatgattgattgatctccaaagtttcccaagaaaagagaaaaagtaggaggggaaggaagaaagaagtgagcagaagttggcttcagagagataggaagggccagttcccttctctgtttaaggaggggagaagacttagatgcagggaaactcattttgacatgccaactcaaaccacagaagccagagtcagttcagctggtttcagtagtaactcaggaccttgacaattctgttaagccatgtcctttaaaagccagctcttattaagtagggggatgaagggggaagcccttcagttaaacaaagttactgacctgtcttttagacaggcaggacagaggatctcctcatgaggtttttttaacctttttgttctgaagttaatgtgtgttttaaatgaataatcttatttataacaacattttcagcgtggcaactgcagtttcagaatggtggaattataccagtcagagagagatgcaaatgatttaaaataggaagaaagcaggtgtctggcccagaggaccagattaagaagaccccatgagagttacaatagttagtgaaaatggtgcttctgcaaacctcatgtctacagaagctggtcaaagaccagatgccaccaggtagaatcccagcaaactcttcaatggtagatatctgtcaccatccttaggaggtaggtctgtggaagctcactataataccatttgcttaacagatatacaaacatggatcagatataatttcaacccacaaggagctcacagtctggctcttcttttgaggcaaaaagactaaagctgttgcctttatggtaagataaatctgccttggagtcaatccaaggtgatttaattactgtaggtagttgtaccaataaggcttgtatcccagagaactccatattgcatttaagcttgttagtaagccaggccagctctatttcctgtgaggactttaaaaaaaattgcattcacacatttaaaaattagcataagcagggtgcagtgatgcacgcctgtagtcccagctacttggtaggcagaggtgggaggatcacttgagccccagaagttcaagaccagcctggacaacaaagtgaagtgaagactgtctcaaaaaaattaattatgacataaaatgttatatgtcttaatttacacaaatataagtatatacttttaaatcccattcttacacacaaaagctagcatactgtgtaaagtattctggacctacacacctttatttttttaacataacaatatgtcttggatacattttcctatttgtctatagaaagtttcatcattattttaaaaaatttgtttttggctgggcacagtggctcatgcctgtaattccagcactttgggaggccgcggcaggcgaatcaggaggtcaggagtccgagactagcctggccaacatggtgaaaccccatctctactaaaaatacaaaaacttagctgggcgtagtggcgggcgcctgtaatcccagctacccgagaggctgaggcaggagaatcgcttgaacccgggaggcagaggttgcagtgagccgagatcctgccacagcactccagctggggcgacagagtgagactccatcttaaaaaaaaaagtttgcttttatatttcatacctgcatggtattctattgtgtgagtaggccatcatccagtggtcttcaaactttcctgtgtactccgtaacagaatttgttgttttgacatgcagtctcactctgttgaccaggctagagtgcagtgccacaatctcagctcactgcagcctccacctcctgggttcaagagattctcatgcctcagcctcttgagtagctggcgcatgccaccatgcttgactaatttttgtatttttagtagagacgggatttcaccgtgttggccaggctggtcttgaactcctgacttcaagtgatctgcccgccgtggcctcccaaagtgctgggattacaggcttaagctaccacgcctggtcttctaatagaatttttaaaaactgtatttacccccttgcacatttaaattgacatctaagatttttattataagtttaaatagttgtaaaggatgtattttccattatattttatacattccttaattttatatatatatatatatatatatagttttttttttttttttttttgagatggagtctcactctgtcacccaggctggagtgcaatggtgcgatctcggctcaccgcagtcactgcctctagggttcaagagattctcctgcctcagcctcccgagtagctgggatcacaggcacccgccaccatgcccggctaatttttgtatttttagttgagatggggtttcaccatgttggtcaggctggtctcgaactcctgacctcaggtgatcccccccacctcagcttttcaaagtgttgggattacaggcgtgagccactgtgcctgccctcccttaattataatttaaaaaatttttgcagatgtagatttatataaattggcaagccagttctctttatcaaattaactgatcaaaaaaaaaaaaacagattaactggtcaaacatagatttactttctgtcagtaaaattttgacttcatttttttaaatcaaataagcatgttaacccggtattgctgtgacaacctttatcctaatttcttttcttcttcttcttcttcttcttcttcttcttcttcttttttttttttgaaatggagtcgtgctctgtcacccaggctggagtgcagtggcacaatctcggctcactgcaacctccacctcccaggttcaagcaattcttctgcctcagcctcccgagtaactgggactacaggcatgcaccaccacgcctggctaatttttgtatttttagtagagatggggtttcaccgtgttggccaggctggtctcgaactcctgacctcaggtgattcgcccaccttggcctcccaaagtgctgggattacaggcatgagccatcgcacccagcttttttatcccaatttcaagatgggtgggcaggtaggtaggtaggcaggtgggtagatagatagggccttcagacttgcctcgcattcatcttttacataaaataagacagtttcaccttgaacacttctgtggtgagactgtttagtcttgtagctctaaactccagccatactctgatgactcccacatttataaatcccagatttgtatactcaactacttatctaacaggtcaaaactgaactcttaaaattcttacatttgtgtccacacatgattggctaagctaatggggctcagatttaccctgtcacctgaaacaactgtaaaactggagaaaatatgtcaaactctgaagttattaggtctaaggtgattaagaacagcgaatcctgagacctgagaaatagagccctatgattgctctgatttaatgctcggagaaagtttccagaccgcggtgcggggagaaggaacatcagcaggccccagcagtctctcctcattgaagaggtaaaatgaaaatccagggcagctggggcagctagagttcacaggcagagaaacaaagaggcaagagatgcacagagaagagaacactggcctgcagatgcccagggagcccactcgagtattcggttgagtactgatcagtgcacccatgtgaggaaaccccttagttcagggaaggagccactcaaaaggattagagagaaccatttacggagttcacacagggccagaaatagttcctgttcccagtagcagagtagaacatttcatgattcaggggtattgattggagtactaagaagagttgtattagtgaaggttcttgagagagagagagagagagagagagagagagagagtgtgtgtgtgtgtgtgtgtgtgtgcacgtgtgaagaaatttattgtaaggaattgccacgtgattatggaggctgataagtcccaaaaatctgcagggcgagtcagcaagatggagacttaggagagccaatgatgtggctatagtccaaaggctgacaggctcaagacccaggcagagccaatgtttcagttcaagtccaaaggcaggaaaaaagttgatgtcccagttcgaaggcagttaggctggaagaattctcttttactcaggaaagggtcagattttttgttcaattcaggcctttaacagattgaatggggctcacccacattagggaggccaagctgctttacgcattttgccagactaacgtttgaccaaagatctgagcacgtatggcccagtcaagttgacacataatgttaatcatcacaaaagtcttgcctcaatagttgggaaaaattaatcctagaataaatgctattctggtcttgcttagcaaaggttaagagcaagaccaaaaagatcaaaccttttccaaacaaaagctgaagaatatttttagggatacaaaaccatctaggaccgagtgtggtaaaatgcacaatgtttaccagccattccagaattactaggtatgagcagaagtagtaacatacaaccaataactgaccagaaatgagacagattatataagtagtacattacagacattagtcattataattgtattccatgtgttcagtaagctaaagaaaagatttaatatgttaagtaaagacatgaaagatgatggctgggcatggtgcctcacgcctgtaatgccagcactttgggaggccaaggtaggtggatcacctgaggtcaggagttcaagaccagcctagccaacatggtgaaaccccacctctactaaaaatacaaaaattagctgggtgtggtagcgtgcacctataatcccaactatacgggaggctgaggcaggagaatcacttgaacctgggaggcagaggttgcagtaagccaagatcgtaccactgcactccaacctgggcgagagagcaagattccgtctccacaaaaaagaaagatacagagatgaaaaatacactgaagactgtcagaataaacattgcacaagtaaagatttgtgattttgaagacttaaccatagaaactatccaaaatgaagcagaaaaaaaaaagaaaccctgaaaaaaatacatgaacacaacatcagtaaagtgagggacaacatcaggtagccaaatgtgtgtggaattggagtccctgaaggaagggagggtatgagggaccaaaaaatatatatagagagagaaaaaaaaatggctgaaaaatttccaagtgtaatgaaagcagtaaacacacagatccaagaatttcaaggaactccaagcacaggaaacatgaagaaaaactatatactaaggcacatcttaagcaaattgcttaaaaccatggtttaaaatggccaaagaaaaagatacattatatacttaagatcagggtgacaccagatttctcattggaaacaatgtaagccaggttaacagtggaacaatacatttaaagcaaagaaagaaaacagttgtctacctagaattcttttctcagtgaaaatacctttcaaaaatgaaggcaaaataaggtctctttcagactttaaaaaaaaaaaggaagaatttattaccagtagatatgcagtacaagaaatatcaaaggatgtaattaaagcagaaagagaatgataactggtagaaaactggagccaccaggagtcccaggctgcagtgagctatgatcgcaccactgcattccagcctgggtgacagagtgagaccctctgtctaaataaacaaacaaataaaagaaaactggagctataaaaataaatgaagacaatgcaaagggtaactaagtggataaatacataagatattttttctttttgtttcagtctcattaaaagatatgtcttagaggtaattacacattgtagcatttgtaacttacatagaagtaaagtttatgacaatagcagcacaaagactgggagtgggagaacaaatatactcttgtaagctttttacactacatgtgaagttgtataatgtcaattgaaggtagactgtgataagttaaagatgcatatgataaaatctatagtagcagagttatagctaatgagccaacaacaataaaaagttgggatataatcccaacactttgggaggccgaggtgggtggatcatgaggtcaggagttcgagaccagcctgaccaacagggtgaaactttgtctctactaaaaatacaaaaattagccgggcatggtggtggcacctgcccgtaatctcagctactcgggaggctgaggcaggagaatcacttgaacccaggaggcagaggttgcagtgagcggagatagtgctactgcactgtagtctgagcgacagagtgagactccgtctcaaaaaaaaaaaaaaaaaaaaaaaaaagttgggataaatatctttaaataatccaaaagatggcagaaaaaaaaactaaacaggaaaagcaaacagagcagatgggacaaatatgaaacaaagagcaagatggtaggttaaaattcatatcaataattaattcaccattaagggccaggcagagtggctcatgcctgtaatcccagcattttgggaggtggattaggaggatcacttgaagccaggagtttgagaccagctttggaagccaagtgagactccatctctacaaaaaaaaaaaaaaaaaaaaaaattagccaggtgtggtggtgtagttccactcaggaggctacggtgggaggatcacttgagcccaggagtttgaggctacagtgagctctgatcacaccactgcactcctccagcctgggtgacagcgagattccatcttaaaaaaaaaaaatcctcattaaaagcagacattgtgagattggataaaaaagcaagacctatttatatttatatgctgtctacagttatcccactttgaatataaaaatacaaatacgttaaaagcaaaagagtggaaaaagatgttaatatgttaacattaatgaaaagaaaaccttagtggcatattaaacaaactatattttaaaatgaatgtcaccaaggataaaggagaattatttcataataataaagagatacattcatcaagacataattctaaatgtttatgcatgtattaacagagattcaaaatacaaacttttcaaggagaaataaatctgtaattatagttgaaggtttaacaactctctcacaaaaactgatagaacaagtagacaaaaatcagtgaggatatagaagatttggacaatataatcaactgacttgacttaattgatgttcagtagaacattcgacccctaaacagcagaatacacgttcttttcaagtgcatatggaactgtttccaagacaaatcattttctgggccattttaaaagcctcagtagacccggcggggtggctcacgcctgtaatcccagcactttgggaggccaaggcgggcggatcacctgaggtcaggagttcgagacaagcctggccaacgtggtgaaaccccgtctctacgaaaaataccaaaaattagctgtgtgtagtggtggatacctgtaatcccagctactcaggaggctgaggcaggagaatcacttgaacccgggaggcagaggttgcagtgagccgagattgcactactgcactccagctgggcaacagtgagactccgtctcgaaaaaaacaaacaaacaaaagcctcagtgaatgtaagctgattcaatttgtagaaagtatgatctctggccaggtgcggtggctcacgcctgtaatcccagcactttgggaggctgaggcgggcggatcacgaggtcaggagatcaagaccatcctggctaacatggtgaaaccccttctctactaaaaatacaaaaaatcagctgggcatggtggcgctcacctgtagtctcagctactcaggaggctgaggcaggagaatcacttgaacccgggaggatgaggttgcagtgagccgagatggtgtcactgcactccagcctggctgagagagcgagactccatctccaaaaaaaaaaaaaaaaaaaaaaaagaaagtatgatctctggccacaaggaattaaatgaggaattaataacagaaaggtatctggaagatctttcaaatatttggaaaataacataattttaagtaaatcatgggtcaaagaaaatcaacagcgaagtaagtaagtatctggaactgaatgaaggtgaaagtgccacataaaatttgtgtgatggagctaaagggaaatttatatagtgcagatggtccctgacttatgacgtctcaacttacatttttcaattttatgatgggtttttcaggatgtaacccaattttaagttgagcatctgtatcagaaaagaacaaaggtcccaaatcaatgaccttggggtcccacttttagaaattagaaaaaatgaagactagtgaaaccccaattaagcagaagaaaaagccagtgtggccaggcagggaggcccatgcctgtaatcccagcactttgggaggctgaggcgggcagatcacaaggtcaggagattgagatcatcccggccaacatggtgaaaccccgtctctactaaccatacaaaaattagctgggcgtggtggcgcttgcctgtgtaatcccagctactcaggaggctgaggcaggagaatcgctttaaccagggagtcggaggttgcagtgaaccgagatcgcgccattgcactccaccctagcgacagaacgagactccgtctcaaaaaaaaaagaaagaaagaaagaaaaagaaaaggccagtctggaaatcaatgaaacagaaaaccagtacagaaatcatgaaaccaaaagctagttatttgagagagcaatgaaactcataaacctctacccagactgatcaggggaaaaaagggaagacaataactgtaatatcagcagtgaaagagatgacatcacacagattctaaagatattgaaagtgtaataactgaatattgatcagcaatctagaagaaacagattccttgaatgacacaaactaccaaagttcactcaagaagtaaccatctgccgggcgcagtggcttacgcctgtaatcccagcactttggtaggccgaggtgggcggatcacgaggtcaggagaccgagaccatccttgctaacacggtgaaaccccgtctccactaaaaatacaaaaaattctccgggcgtggtggcgggcgcctgtagtcccagctactcaggaggctgaggcaggagaatggcttgaacctgggaggcggagcttgcagtgagccgagattgcaccactgcactccagcctgggtgacagagccagactctgtctcaaaaaaataaaataaaataaaaataaagtttagtaaaataaaaaaaatcagtaaaatacaaataaaatttagtaaaaataaagctgattctaaaatgcatgtgcaacagaggatctagagtagccaaaataagctcctctgtctgacttaaagtcttaattaaaataatcagacaggccgggcatggtggctcacgcctgtaatcccagcactttgggaggctgaggcaggtggatcacctgaggtcaggagttacaaaccagcctggccaacatggtgaaaccccatctctactaaaagtacaaaaattagtcaggcgtgatagcacacgcctgtaatcccagctactcaggaggctgaggcaggagaatcgcttgaacccgggaggcggaggttgcagtgagctgagatcatgccaccgcactccagcctgggcgacagagtgagactctcaaaataatgaaataaaaataaaaaactgaacttcatatcttgtccccagatctgatatagtcacttcttcctttgttcagttaaacccagcttcatgtttctgcttgcccagggccaataactttgggatcttccttgactcttctttttctgtcaaaccccgtaccagtctttcaggtaaccgtaacttcagagtctatccaggtcacctctgcttgtccaagcccctgtcatctctcacttggatgatttcattagcctcttggccaggatccctgcttctgctggtatttccttagtttcttctccctacagcagccagagctaatcattttaaatgtcatatttctgtctcgcctcagctgcaaaccttccagtaccttcccagctccttctaaataaacaccagaggccgggtgcagtggcttatgcctgtaatcccagcactttgggaggccaaggtgggcagatcacctgaggtcaggagttcgagaccagcctggccaacatggtgaaaccccctccctactaaaaatacaaaaattagctgggcgtgatggcgcctgtaatcccagctacttgggaggctgaggcaagagaatcgcctgaacccaggaggtggaggttgcagtgagccaagactgcgccactgcactccagcctgggcaacaagagtgagactctgtctcaaaaaatataaataaataaacaaacaagcaccagagccctggcagtcacctgtgaggcttcaccccgtctccccacccactataacctctgacttacctccttttacactccttcttgctgactctgttccttccacattggcctctttgctcttgcttgaatgatccccagtcatcctccctgcccagggcctttgcacttgctgttctctgccaggaaggtgcttcctccgtagttcactcttcacctcctttgggtctttgctcagttgtcatcttcagtgatggctcttctgatcaccttatttaatttttattttttaattaaaaattttttttgtggagatgtcttgctatgttgcccaggcttatctcaaactcctggactcaaacgatcctcctgcctcagcttcccaaagtgctgggattacaggtgtgagctaccacacccggccccgatcaccttatttaaaattgcagctcccagcccccaaccccagcatacccattcctcttttttttttttgagacagaatcttgctctgtctcaaattccctccagccatgagccctgaaatgaaacaagttatctatttccaaaacacaacagtggggcaggcataggatagacattcccattccaaaagggagcaataggcaagaagaaaggggtagctggtcccaagtaagtccaaaacctgagacagagtcttgctctatcgcccaggctggactgcagagtggtgcgatctcagctcactacaacctccgcctcccgggttcaagcgattctcctgcctcagcctcccaagtagctgggattagaggcatgcaccaccacacctggctaatttttgtagttttagtagaggcagggattcaccatcttggccaggctggtctcaaactcctgaacctcaggtgattccccccgccttggcctcccaaagtgctgggattacaggcgtgagccaccgcctggtctccctcattccttttgttatcagtcatccctcccagccctggcaactgctgctcactttcctgaccttagaattttgcttattacagaatgtcatataagtagaattataaagtatctaagcttttaagtcctaatgcatttaagatctgtaaaaagtaaagtagaggttcctcttcaaagactttcctccccatctaattaggaataaatagtaacttctcttagaagaaaaatttattcaaagacctgtgctaacattcttaaatatctgctagccataataaagaaatcaatgtactttatattcttagctctcacaatttagcctaaatatttgccctggcatgcttatactggtccaagcaagcattaggtcatggcctgttcctcttccttatttgaaggtgtttttacctttctcagcactccacaagttacttcctccttcctttgttctcctctgcctttgcctcttttaaaaagttctaagttcctagccaatcaggacaaatacagaatgtgaggtcctgttccagccaatggaaactggacacagcagtagggtggacgcgtcaggttgtaaatgaccctgtctcctttgttcagtgtactctcatggcaaaactgctggtgagtgtaccctttctgcagaaagtaaaaaatggccttgctgaggaaattaaatttatgttcaagtgctttttcttcagggcaccagggaacaagcatttctaacagatccatatgtgtcgctgcatgtaatcagtggttcattcctttctattgctgagtagtgttccattgtatggatgtacccgtgtattgatccatccatccactgaaggatatttgagttgtttccagtttttttgtgattatgaataaagccactgtaagcattcacatagaggctttggtgtggacataagttttcatttctcttcggtaaataattaggagtagaattgctaggtcgtaggataagtatatgcttaacttaggagaccctgccacagtattttacaaagtaagggtgctattttgcattactaccagcagttcacaccatatgtattcacaccagttgctccatttggtcttcgttctgtgacttgtctctattctcttaacagtatctttagaacagcagaagtttttgattttgatgaaattcagtttatcgggccaggctgggtggcttatgcctataatcccagcactttgagagaccgaggagggtggatcacttgaggccaggagattgagaccagcttggccaacatggcgaaaccctgtctctactaaaaatataaaaattagccatacatcgtggcgcatgcctgtagttccagctactcaggagtctgaggcaggagaatcacttgaaccctggaggaagaggttgcagtgagctgagatcatactgctgcactccagcctgggcaacagagcaaaactccatttcaaaaaaaaataaaataaaatttcagtttatcagtgatttctcttaagtttcatgcattgggtattgtagctaagaaatcttcacctaactcaagtttacaaagattcttctgttttttttctagaagatttataagttttaatttaacttttaggcttataattcatttcacattaaatggtgtatgtggtgccaggtattgctcgagagtcatcatcttgcataggggtttcggatgttctagcaccatttgttcaacagatagcctcttttcattaaattgccctttcacctttgtggaagatcaattcagcatgcatgtatgggtcacttgtatgtttttaattgatttctaaattctgaaaggaaaatgataaaaaggaagattctttcactatatctgcatttccaatttctcccccaagaacgctttcctaacacctgcaggcagaactaaccactctgggcccctgtagctgttgtgcgtatgtttattattgagtttttctggcctttgcagttctttcagtgtctgattctccctctctctcagcttctttctcaagactgtgcatccttggaggtcagaaattgtctcatcctgccgggcgcgatggctcatgcctctaatcccagcactttgggaggccgaggcggttagatcacaaggtcaggagttcgagaccagcctggccaacatagtgaaaccctgtctctattaaaaatacaaaaattagctgagcgtggtggcacatgcctgtagtcccagctactcgggaggctgaggcaggagaatcgcttgaacccaggaggcggggaggttgtagtgagctgagattgtgccactgcactccagcctgggcaacagagcgagactccatctcaaaaataaataaataaataaaagacaaaataaattttctcatccttttttgtcatcttcatcacagctggctctctaatgttcgttgaatgaaagggccattcccaaatctctgataagtcaagcatttttaggagagctaaaccagccaaatgagaccaggaagtgaagaaaaatacctttttttttttttttttttttttggagacggagcctcactttgtcacccaggccggagtgcagtggcacgatctgagctcactgcaacctccgcctcccaggttcaagcaattctattgcctcagcctccccagtagctgggactacgggcacacaccaccatgcccggctactttttttttgtatttttagtagagatggagtttcaccgtgctggccaggctggtgtcaaactcctgacctcgtgatccgcccgcctcagccttccaaagtgctgggattacacgcgtgagccaccgcgcccagctgaaaaatacctactttttttttttttttacagcaggaggtgtttctgcttctactttgctgtgacttctgtttctccaggcattcctggtatggacatttataccaggtgttttggtcattggtgggctctcagcttcagaggacacttggcttctctataccctatctttggtgacttctgcttgttggacttcagtcactgcagttcacaagtttcacactcatgaaatatttattgttaattaattccaatatttaattgattgcttctctttctcagcactttggaaacatgtttactacctagacattttcctacagtagatttttaattcttaacattttgatcttaagcaatttaccatgatacttggtttccatgagagatttaacagttggaactgtactgcccataagatgcaggtggggtcatggtatgtagttgggaactctacaaatggcagcccttcatgcctttgcaaaggctgcaggttggaaagctgccattacttgtgagggcgtccaaatgcttcctgtgaatggaagatgtgtttctgtgcctcactgtcttcactgtgctggattgctggctttctcagatgtctttatttgagtcaacctgtattccatctccccactcccacccagtcttgatcatctcatttgtgtgtatacaaccttcccctcacacacacctctttgcagagtttcgttgcactgggcgttgggaggagaggcgtcagagttgggtggaatcatttcaggctgactgtccagacatagctctcattagaagcatcagtaaataagctgaaaaccaacaacacgtacagcacagtctttctgtgtttgcagcttggaactgagcagccctcactgggtgcatttctaatcctttgtctcatcatttatatgacatccacttagaaaaaggtgttaaaatcatataacatattgctttttccccttttttattagcagttttactgaggtgtaatttatatgccataaaatttaccatttgaatgttcaattcaatgatttctactaaatttatagagtgtgcaccataaccacaatccagttttattttttatttttttttcgagacagagtctcactctgtcactcaagctggagtgcagtggtgcaatctcggctcactgcagcctccacctcccgggttcaagcgattctcctgcctcagcctcccgagtatctgggactacaggcacgtgccaccatgcccagctaagttttttgtattcttagtagagatagggtttcaccatgttagccaggatggtctcgatctcctgacctcatgatccgccagcctcagcctcccaaagtgctgggattacaggcgtgagccaccgcacctggccccacaatccagttttagaacatttccagcatccccaaaagatcccttttgtccctttgcagcagtcctacccccacttccagccccaggcgaccattcatttgctttttctatagatttgtcttttccgtacgtttcatgtaaatgaagttttatacaatatatgaccttttgtgactgttttttttcccagcatgtttttgaggtttatctgtattatagcacgtctcagtactttgttccctttttatcactgagtattattccattgtacggacatacatttttctctattcactagttggtgaacatttggattgtttccagttttggtcatcattaacaatgctactatatatatccatgtataagtctttgtgtggatatacattttcatttctctagggtagatttctagaagtaaaattgctgagtctttttttttttttttgagatggagttttgctcttgttgcctgggctggagtgcaatggcataatcttggctcactgcaacttccgcctccctggttcaagtgattctcctgcctcagcctcccgggtagctgggattacaggcatgtgctaccacacctggctattattattattattattttttttttttttgagacagagtctcgctctgtcacccaggctggagtgcagtgcgcgatctcagctcactgcaagctctgcctcccgggttcatgccattctcccgcctcagcctcccaagtactgggactacaggcacccgccaccacccccggctaattttttgtatttttagtagagacgaggtttcactgtgttagccgggatgctctcactctcctgacctcgtgatctgcctgcctcggcctcccaaagtgctggcattacaggcgtgagccaccatgcctggcaaattttgtatttttagtagagacgaggtttcaccatgttgatcaggctggtctcaaactcctgacctcaggtgatccacccaccttggcctcccaaagtgttgggattacaggcgtgagccaccacgaccggtcaaattgctgtcttatagtaagtttatgtttaactttttaagaaactgccaaactatttcctaaagtgactgtaccatttacattcccaccagcaatgtgtgagtgttccagtttttccatgtccttgccaatagttggttttgtctgcctacctatccccgcccctgcttttttttttttttttttttttttaagagacagggtcttgctgtgttgttcaggctggcctcaaactcctgggctcaagtgctcctccctagtagctgggactataggcacacaccagtgtaccaggggttgtctgtcttttgattatagctattggaatgggtgtgaagcagcatctcgtgattttagtttccatttcttgaatgactaatgatactgaacatctttttatgtgcttattagccatttgtatttcatctttggtgacctgtcaattcaaatcttttgcctatttttttttattaagtttgtgcactgtcttattatcgagtggtaagagttctctgtattgtgggtacaagttcttttttttttcgagatggagtctcgctctgtcacccagggtggagtacagtggcacgatctcagctcactacaacctctgcctcctgggttcaagcgattcttctgcctcagcatcccaagtagctgggactacaggcttgcaccaccacacccagctaatttttgaatttttagtagagacgggatttcaccatcttggccagacgggtctcaaactactgacctcgtgatcctcccacctcggcctcccaaagtgctgggattacaggtgtgagccaccgtgcccggccttccaagttctttatcaggtatatgatagcacatgtagctttttatcttccttacaccagtgcaagtttatagaaaggaaaagggatagttaatagtaatcataataatgtttaatatttgaatgttttttaggagctggacactgggctgcatgtttcacggggatgatttgggtcactcttcaattttctaaaggttacaggactgtttcaaacacctctccttctattaacagttttcaatggagaaagaaaacaaaagattgatttggggaattcttaggttaatgcgagttagaactgaccttttaacattattaccataaggactaaatggctgttacagacccaaaataggagcaattcaaacaagcgagatgcttctctctgtaacaggctgacagtcgcggtcctggaggggcacgatggctcctcagggtcagacacctcctgtcccttactctgtaaggcacagcttctacctcaggatccaaaatgcccacgccagctgccatggcccctcacaggcagtggacaccagaaagaggagagggagtgcgtgcctgttgctttgaagaacctgacccagaagtcttacacagcacttttgcccacataccatcacttagtcatgtggtagtgcctggccacaagagagactgggaaatggagtctttattctggggtgatgtgtcccatctgcaactgaggggttaaggcctaaaacttagggattgaaggcaggagaaaaaacagatgtcgagagccagtgttgggattgcagggtcctgtgtgggattcctgttcattttcatcaaagaaagtctagttgatatgcaattgttggcctaagtgttattgaggagttaattttactgttgaattacaccagaacatttgtcatgtgaaataactataaatacgactgattattgcctagtgtattacagttctctccagagacacaggaaacaatagggtctgtctatcggaatagatatcctaaaagtgtatcttttgaagagattgattataaggtattggctcatgagatgatggagcttaagaagtcccacagtctgctctctgccagatggagacccaggaaagctgctggtgtagtttgaaggcctgagagctgagggccagtgatgtggattccagtctgaaggtctgagaacccagagggctgagggcaggagaagatccatgtcccagctcaagcaggcagagggagggtaaatccaaccttcctttttctgtttttttgttctaatcaggccctcaacggactggaggatgcccagccaaagtggggagggccatctgcttaggcagttcaccaattcaaatgctagtctcttctagaaacaccctcacagacacacctggaaataatgtttaagatctgggcatcctgggggtctagtcaagctgacacatacatttaaccaccacacttagggaatatgaaaataacttagacattaaatttgattttggtgttgctgtaaagaatacagatcttaggcaccagctcgtaggaggggtggtaaacaaggaggacagcttttaccaggttgcctttggaagcaaaactctgctttgctgttttgttttcaagcggctgcatggccactgagcgagccctttcctgccatcaccacagtgccacttagtgaacagtttctccaaggttaactgtagctgctcttgttcagttaatatgtagttctggattaatatgccacatctgtcactttaaggaagaaaaagagcagttccgtttagagatcccattgctcctctaaggacagcagtcatttctaacaagcagagggaagaaatcttctggttccatttgtcctttgtgtgtgttgaaaaccttgacaacaaaacaaattggtactgaatggtttgaagaaataaacattaactcccctttagtatttcatttctagggtttccagccttggtaacaattaccaagaactttgctgtcatggggtacagctctaggctgctgacaggatcttcatactggaggagactatacttttatagccaattccaactgccccagagataacttggataaacactggctcctccactacctcctaatccctgctgggttttacactgtggggaggtttagacaacttgttttctttcccttttagcccttctctctacactcccctccagctgccttcttcccccacccccaaatgtaacgtgatctcctaggcctgctctgctgcttttgcagattcttttagtcttagagaaagagaggaaaacattaaccattggtttaccaattcaaagttaacaacattttaaacaaaaggagcttgtcaataaccagggcttgggcttttcttttatcagttcctcctacttggctgagtttacctcatcataaatcttatcacacagataataatttgacagctttagggtttttgcagcattgctattttctattgcaacaaagctgtaacattgctatttttgatgtatttgttttatagatatcttaatcttcaatattcagattaaaaacaaacaagacttagtgatttaaaattatttggtaaaaaggtaaagggacagagacatctcatgaaaacatggcaaaatctctttaagtttaattcaagcacttagagtttaataagaatatcccaaaccatttttgcttaataatatttctgtttataattaaaccttaaggtacagtgttgtaactggatttaagaatttagagttttgttgtttcttcgccatcagcgaaattaagatttaaaacctatagtttgcaacaaattttagtttttcagcctagtgtcctaaaacttgtctgtcatgggtacctagtaggtgtcatgtcacaggaaagggatctaacaagggagtttgaagataggggttttccttgctctgccactaacttgctatgtgacctagggcaagttatataacctatctgtgtcacagttgtctgaaatgcaatatggggaaaataaattcttaaaatgacaataaaaagatttctttttttttttgagacagagtcggcgcaatttcggctcactgcaacctccacctcctgggttcaggcgattctcctgtctgagcctcctgagtagctgggattacaggcacacaccaccacgcccagcaaatttttgtgtattttgtagagatggggtttcgccatgttggccaggctggtctcaaactcttgagctcaaacgatctacctgccttggcctcccaaagtgctgggattacaggcatgagccaccacgcctggccaaaatatatatatacatattttaagtgaagatgtgtaaggactaaaggagcccaaaggagacagtaggggataagaaaggcaaagagagtggtaatagatttagcagaatcaaggatgccaaaacctcttctgagagagaccttgattaagagtgagctggtttgcctgtaagaatctgcggaaaggtcagaaattagagacattagtaccacagaaagcaggggatgaggcctgaagctgaaatcaaggggatcaattgagagcctataaaaggagggtttagattcttcaaacccctcccaaccccacagagtcagatgaccacctctcctaaccacctacaggagaccagaggcattatattgagaaatgcccggccgggcgcggtggctcacgcctgtaatcccagcactttgggaggccgaggcgggcggatcacaaggtcaggagatcgagaccatcctggctaatgtggtgaaacaccatctctactaaaaatacaaaaaattagccgggcgtggtggcgggcgcctgtagtcccagctactcaggaggctgaggcaggagaatggcgtgaaccctgaaggcggagcttgcagtgagccgagatggcgccactgcactccagcctgggcaacagagcaagactccgcctcagaaaaaaaaaagaaaagaaatgccccgtagaggctctgcactcagaagcctgacccccagcatgggtccgggtaggccattggccgaaaatagtggagtgggcaaagagcagattttttaaaagtctgcatactgaatagtaagattcccagccctcttctaatacctgctagccaggggcctaataaggaagagattgaaatattcttttctagggggaaaaagttgaatgaccctagagaaaagccctacagataccaacatttagggattctcttgaaaaagcgagtttaccatctcctttcactgaagcttaccagtggctgtgccccttccacgcatacaaaactaacagtcatcttttagttgctcattcatagagagtgagtggcctgccaaatctctctaggcattagaggaaggtctctaacctgatattaatagataaagggtaaaacaaatagcagaaaacaggaacttggtggaaataaagacaatgcagtcaacagaggaagagtttaaaagaactattcagcttgggcaccatgactcacgcctgtaatcccaacactttgggaggctgaggtggacagttcacctgcggtcaggagtttgagaccagcttggccaacatagtgaaaccccatctccactaaaaatacaaaaattaactgaacatggtggcaggcacctgtaatcccagctattgggaggctgaggcaggagaattgcttgaacctgggaggcatagcttgcagtgagccaagattgcaccactgcattccagcctgggtgacagagtgagactctgtctcaaaaataataaataaataaataaataaataaataaataaataaagctatgcatttcccacaaatactgcttaaaaaaaaaaagaactattctttgctatccttggagagagagattgcaaatgtcagaaaacaggatgcttttaaaaacaataacaatcagaataagaataaaaatgaattatttttcttttatttatttatttatttatttttgatacatggtctctgtcacccaggctggagtgcagtggcaagatcacagctcactgcagccccaacctgcctcagccccccaagcaactgggactacaggcacgcaccaccacacctggctaatttttaaattttttgtagagagatggggtctcactatgttgcccaggctggtcttgaactcctgggctcaagtgatcctcctgcctcagcctcccaaagggctgggattatgggcatgagccaccacaacccagcaagaatgaattgtttaatattaaaaacattatagcccaaatttaaaatccaacagaagatgtggaaaattgtctcagaatgtattgggttggtacaaaagtaattggggcttttgccattttaatgacaaaaacgttgattacttttacaccaacctaatagaacataaagacaaagacatggactgttgagagaacaataagagagaacaggtaagaaaattagaaactctcgggaggctgagacaggataatcgcttgaacccaggaggcagaagttgtagtgagccgagactgtgccattgcactcctgcctgggcaagcaacaagagtgaaactgcgtctcaaaaaaaaagaaaagaaaattagaaactcaatcccagggctttaatatcctttttgtaggaattccagaaagaaaacaaaaaaataatgataataacgaagaaacaaatggaggagaagttaggagacgttaaatgtaaggaaaaatttcagagtttagtgaagtgagtattttccaaaacagaattatataaatctctggatttatgagtgcccagaataatatatgaaaggccatatcattgtgaaagttcagaacaccaagaatattagaagatcctaaaagtttccaaagagaaaaaaagcaagtcattttaaggggtcaggaaccagaatgggattgcatctctaaacaatacttactggaaactagatagtagcagataaataaatgccttcaaagttcatttgggaaaattattttcaacctagaattctatacccagacaaactgtcaagatagaagaaaggtatttttttagacatgcaccctccctccccacctctgtgaagctactggatgatgaattccagcaaaatgaagaagtcagtcaataagtagaactgagcccgggcacggtggctcacacctgtaatcgcagcactttgggaggccgaggcgggcagatcacgaggtcaggagattgagaccatcctggtgtaacacagtgaaaccccgtctctactaaaaatacaaaaaaattagcagggcgtggtggcgggcccctgtagtccgagctactcgggaggctgaggcaggagaatggcgtggtgaacccgggaggcggagcttgcagtgggcggagatcgcgccactgcactcccgcctgggtgacagagcgagactacgtctcaaaaaaaaaaaaaaaaaaaagtacaactgagaatatttgaaatgttttagtgattggaaaaaatatcactagatgtttgacagatctttgaagtatttgagggaaaatggtgataggtatatagaagactaaatgaaaacaaggcaatatctctaggataaacaaagatgtataataaaggaaacattcatagtatattattggctaagcagtgaagaatatttatatcatcctaataatgtaaacactaactattgatttatcccccaaattgtgattttattgttttggggggttatggatggtgatagtataatactgctgtcataagttaatagataatggataaatctaatgaattcagaaagagcaagatgatgttactagctaaaaatctgaaggaggctgtctctgcggaaacatacaggggaaagggattgctgttttttattaccagatttttacaagactatttttagccttttttacaagacttttagtactgcttgagttttaaccatgttatgtatattactttgattaaaaagaaaaattaatttaaaaaaatgagcatactaataccattaattttctccttctagagaaaaatgtttaacagttaggtttagacttgttaattataaaaatatagtagtcttactgtaatgagattttctagaaagcggatttactctaaggcagttcagatttggtccccagctgagaattatagcctggaaataccaacagaaaaatcagtgtcatttgaaggacagtcatctgtgcagcctgtgcatgaaatcatgggtctgaattaggcccccattcaagatgcgggggtgtggggtttgtgttttgctgaagctgtggttgcaaatctttgctttaggatgaaaggtggggctctctgttacatttgaaccttagttgctactcctacctcacagctagtgtttctttctcttcataaaataaattgatttcctggtcccttggaaaacctccagactgctttctggaatcattttcatatagcatgtttgttaatttgagctcttgactgagtcccagtgagatgtaggcaggaacacagacctgatacagaaatagtcctcttttggaaataacaatagctactgtttatcgagcaccttctgtgctttgggcactctccctacatgattcctaatcttcataataaccctgtgagataactactcttatgactacctcccatttttcagatgagaaaattgaggctctgagaggtgaagtcgttgcccatggccccccagtgagtggcagatctgagactcagcgctagcctttgtgacacataactcgtgaccagcaggtagtgctacccgattgaaaaaatgtgagacttaaagggatgcatgtgtccctatacagtggcaaaaactggtatgacagaaatgatgttttaagctatgttatcctttagttggacatacactggacccacagagctagagtacctggcaggctcttgctggacaatgtttgctgaatcagagaaagtgggctttgttggcatttgcatgtgaaatactgtgcacatacctcaagatctttgctgttccccccctaaagctactagctatgctggaaacagtgaaggggtcagccagacctggggtcaccttgtcctggggtcccaagccaggcctcctgtcaggttcatctgtctctgttccacctgcactcacacccagtcttgctgctgagagatggagcatccacatcctgaatcctgcctccttcccttgacacacttctatggtgggtgcctgcccaactttttcttaataggactaaattccaaattaaaaaaaaaattcagtctctctcctctaatcatccaagaagagtcctcttttggtaacttgttctagaactatgcctatcttaaattcagaaattgctttcaaatgaattaaattttagaaatatccattcctgttcgtcctattcccagtgaaaataagcaactcttccctcttccaaaacagtggatttcttttctttttttctttttttgaaatgtagtcttgctctgctgcccaggctggagtgcagtggtgcaatcttggcacactgcaacctccacctcccaggttcaagcgattctcctgcctcagcctctcgagtagctgggattacaggtgtgtaccaccatgcctggctaatttttgtatttttagtagagacaggatttccccatgttggccaggctggtctttaactcctgacctcaggtgatcgcccaccttagcctcccaaagtgctgggattacaggcgtgagctactgtgtctggcccccaaacagtggatttccatcttacatatgcatacagatcatctgggagcttcagatccagcccattgcacatattctgattctagactagaaaccaagactccgcattttaagcaagccccacattattcaaaatagattgtccatggattatgcttttagaaacattgttcagatcccatgctttaatgttggggagtttatcctttccttctaaatattttttttttttttgagatggagtctcgctctgttgcccaggctggagtgtggtggtgcgatcactgctcactgcatcctccacctcccaggttcaagcaattctctgcctcagcctcccgagtagctgggattacagatgcccgctaccacgcccagctaatttttttgtacttttagtagagatggggtttcaccatctcaaaaaaaaaaaaaaaatcttaaatagctctagttccctgaattattttctcctaagacatgttttcccaacagttaatgttacttatttcatcttctgatcttactcatattatccacctttcacttaaattggaaattttctgccattttacaaagttcaagactttcctaccttcctacctcctggctgtgtgaacttggacagaatacttccccctctcccttcagtcagttttcttacttgtgaaatggaattaaactatacaaaatcattaggttactgtgaggcttaagagtgcctggcacatattacatgcacattaaaaatgtatttcttcccaaactaatgcaacatagcaaacaagttagttcacagtctttaatttctttccttttccctttctttttagctaatagcttttgggtcttaggctgattagaaggaatccttcttggggtgttcttctggtaagaagattgaagctctcctttatttgcttgtaacagaatgctcttcgtatctttcaaaagcgtcaaaagctggatatggaaagtataggccaggtttgatacagaagtgtagtttggcagttttttcagtatccattgccactaattcagtattttgttagatctgttgatgtacggttttataactaaaattaaaaagatttgggtacagtttataagcattattcattttaagaggatttaagaggataagaccattttagactcgagcagaatattagtaataataattagcttaaaaaaataaacatataccaatataaagaacagcacaaggcaagcaaatatctgggcctatattaacagacggtattcaaggatatggctagaatttccaaaatgcctaataatgtgtaattcttggcattaagccgccaatttgaatatggccctttaatatgaaatgctatataatgacagactttattatagtctgatccaaggcccctgaactttccaaaagggcaacaaaacagacagatactgtattttttcactagttggtactggtggcacccttttgagtgttatttttggcattacgtgtagaggttgacagattcaagacaacactaaacatgttagcagtcaccacacgcaggctgtccagcagaatgacagcgcctcccagatcctagaaagcatccagagtcactgcacccatcccctgggggccctgtagtcctgcatctccagatgatgggagctgcaaacttccctgatggtgccctcttggatgctcatgaggcctgatctggtaccacgactctagcagtaggtccattcaatggcagtaaaaatacatgttgtatgttgatcagaatttggcaggacacatgtggaaagttaatgacatcctggccgagaacccctgtgatatacactggttacatcctgtccaactggagtttttatgtgctgtttttggcagagccaagtgaagatcttgttacttagccattcctgaggtactgaagatacccgggtttttgtcattacaggataggctagaaagtagccagggtctcataaccaaggctttctctgaaacatataatgacaatgctagttatttgaccaaagatatccaatgctttaatccacttggaatttattcttgatgtgaagggtcagcatcatccactggtcgggagcctgatccttggagccaggcagacctgggttgagtccatctccgcctgtttccagctgtgggcaaggtgtctgacttctctgagcatctgtggggtttgtttgtttttaaacctgaagaattgggataatctgtttcctcacagaatcattacgcagatgcaatacagttaatgaaggtaacatggcacataggaagagcacaatacacgtcagctcttactcttcatggctagaagatgtggactaaacttccccctttacagttttgtgattaacaccatttatgaaagaaataaatgaaataaattccttttcctgtcatgaggggaacttacctgattttattgaattatttggtgccattgagttccttattgtttccctctgacctagatttctgggtgtgcgccagtaacacgactgtgattgttgctgctttataacattgtctgtggtcagtccaaacaagagtttaaaataattgaatgaaatggattttaaacaatggtccccccaccctcaccccgtggcactcggccaaaggaagaggaaaagttctctgttaaagaaaatgaatcagagaagaggcccagaccgtgcctccgaaaattctcattgtagggactaagtcctctcactctgaactcacacccgtctgttcgcagcctcacctcatatcctggtctggatgtgcggcctcaccggggtgtcgtgcgcacgtgtggttgtcctgtgggtgccagctctgacttcctccccagacacccactgcctcagccttaatcacaggacgcgcgttgagtagaaatgagaccagttagtattggtaactgcagaggaatttgcattttcaccagcgttctcgggtcagagggtttgcctggcaccgcgtactgggaaactcgccaaaagcggtgcaggttggagacgccccaggccgcggtggagttgcgcgcggcttctaaagtggagtggagcaggcctgcaccctccccgccggggctgggacggcgcttccaggcggagaaagacctccgcgggccgcgcgcggccttccccctgcgaggatcgccattggcccgggttggctttggaaagcggcggtggctttgggccgggctcggcctcgggaacgccaggggcccctgggtgcggacgggcgcggccaggagggggttaaggcgcaggcggcggcggggcgggggcgggcctggcgggcgccctctccgggccctttgttaacaggcgcgtcccggccaggcggagacgcggccgcggccatgggcgggcgcgggcgcgcggggcggcggtgagggcggctggcggggccgggggcgccgggggggcgcgcgggccgagccgggcctgagccgggcccgcggaccgagctgggagaggggttccggcccccgacgtgctggcgcgggaaaatgttggagatctgcctgaagctggtgggctgcaaatccaagaaggggctgtcctcgtcctccagctgttatctggaaggtaagcccgggccgcacgggttgggctgagtagccgcgcgccctcccgctgctgctgggcccttcctaggcctcgccgcccgcgcgctcccgcctgcgccctccccgggtcttgtcttttttttctttcttccctcttctcttctcttctcttcagttctcttatattctgtctctctttctttctctctgtgtctgtctcttttctcttctcttgtctctctctttttctctctctctgtctctttctctttctcgcgatggcccctaggcgccgccggcggagcgtggcccccagccccggcaccagccccggtagagccacgccggatggtgacggcggcgtccgggaccccacagtgcgggctcccccgaaaaagtttgagaaagccaactcgccaggccttaacatccctgggatcccacgttgtggaatttccaccgttaattgggactgtgtgttaaaaagatcgacccgtgtttgtgaaaacatgcgatttccattaagcatcatgtacacaaggagcaccactctaccatttatggatacagaatttcaagtgatctttccatctcccccacccccatcttatctacagaccacgaagaggtcttaggaattcgctctcttttcaggggaggggagcccatttaagaaggcgtttccaaggagtcccccagagtactggggtgtcatgttcgtttccttaagtgaagctcagggccaggagggcttcacttttgcgttctttctacctactctccttctctaattacatctgttttattttatttttctttcctgttgtagtctttagttttataaatggaaaaaaagtaaattaagggttatgggtcttcactttcgtagcttctaggtgggtatgaaattgatctgatttgagactggtgctcccagaccattgccttcagcagaaaggctatctgtgcttcagagatggagttgcacttggacttctctgttgttttcaactaagaatataagagtaaaaatgtggccgggcgcggtggctcaagcctgtaatcccaacactttgggaggccgagacgggtggattatttgaggtcaggatttcgagaccagcctggccaacatggtgaaaccctatttctactaaaaatacaaaaaattagccgggcgtggtggtacacgcctgtaatcccagctactcgggaggctgaggcaggagaattgcttgaacccggcagggggaggttgcagtgagccaagatcgcgccactgcactccagcctgggcgacacagcaagactcggtctcaaaaaaaaaaaaaaaaaaaaaagagtagaaagaaatgtgaagctcactttagatcaagtaggatttctgctagaatgccatgtttttctttttaaaaaagcaggagaaataaacaggcctcatttattttctaacctatttttctttccttaacctctgactctaccctgaaggcctgcttggagtcacatatgcagatcgcactagtgatgtgggtgaaacctgtttgcccttcttgccacagtttgtgtacctcactcctcttctgtctctgggattccagggggctgaatggcccagagaaggctcatctaggtggagtgggctcacagagcctagcatttggagggtggagattgtttatttgtccctaaaagttgatgtctgagcattccaaatcatcttcaattaatacatcgcttattttttccaagttgctcaaaattctgagcacattctcatttacatctgtatcgagttagtgttaggcatcatcatctccactttgcagatgggtgcctaaggcggggatgaatggacttttgtaagtgcagagctggagcaaagtttaggtcacagctttgccaagtcattgttttgttttcctgggagtaaaccggagatttagtatggacctggctggtttaattattgttttgggaaaaaaatctaaaactcattccaacatgtcttgaaatgaattataatgttgactctaaagtctcaaaaaaaaaaaaaagttctagtacagcagactgccttgggttaaaccttcttctaggaaggtgtcactctaggaattctttcttactgagttttcttcttttcgttgttatagttaattagagaaagtaaatggaattatcattttgtagttggtagactttaacaagcctcagcgatagtcttttaaagactggaagtgctatttgtcttttgggaaaaatagttcctaacatctggatgagtaatccaggctttccactttcactgacagcctaaatcagtcaatcagtgtcgatcagtcagtcagtgtttattgacgcccactcagggtcccaggcattccctgtcagggagagaattgaatgagatcaagtttttcattgttctatcaaagaagcttccaccataaatgtggtttgatggtgcatgctacctgctactcagcctagctgtcaaagcaacactggtaaacataaagatggctagcaattattaaatgcttactatgtgccaaatactgttttattactctatgtgatctgatatagttatatttcatgatagcactatgattatttccattttatagatgaggaaactgagtcaccaaaaggttaggtaacttgtctagggtcacagagccagtgagtgggggtactggaatttgaactcatgctatctggcttgggagagcccacttttagccactggatgctctactgatgtctcttttggaattcactgtgattttcaaaatggtatctccagaaactcctccaattaatccctgaaatgtaagggtagtctctttttcttttctagaaacctgatcctggcaaaatgattagctttaacagtaggattaattttttttaaatgtatataaaaatacttgtatgtaagaaatcctaaaagggtatttagtgaaaagtaattccctctcctattcattttccccacctagaggcaatcaccctggagttttatgtgtgtccttctagaaatactgtgtgcacatataagcatttattataacatgtgaattttgctctccagctttttgtgaatatgcatgtttatagcagtatctattcaccaggaccgcatttagctttgcaatattcggtctactttgaacactgaattttctgattatttctttcccctggagacgtcaaagaaaacatgaacactagccttttgaattcagggttttcttttcattttattttcatttgttcatgtgtttgtatttacatacattgtgcctgaatattgtacatgtatgtgacagttgtgtactgtagaatcaaaaactaatgtctgtgaactgaactcttcttgaactttttgttgttgttgttattgttgcttttggagatagggtcttgctctgtcacccaggctggactgcagtggcacaatcacagctcactgcagccttgagctcctggtctcaagcaaccctcccacctcagcctcccaagtagctggggctgcaggcatatgctacctgactaattaaaattttttttttttttttgtagagacagggtctcactatttttactagtttgcccgggccaagccagtgttgaactactggcctcaagtgatcctcccaccttggcctccccaaagtgcatccctacaggcatgagccactgcactcagcctgaactttcgaaatttattttaagggcccacttttaaatgcttcttttcagcagctaactttccagcggatgcttcatgtggtgccagccatacagatacgcttttagaacttgagctttggagaagcttatgcttgctttctgctctctcctgaggtcatcagatacttcccttgttcagtaacaaagaaagtgagactctttctgttacctaataaaaggccagtctgtccatttcattttgggtgctattaacattgttgaccatttacaatgtcacaggccctggcactgagcacatcatgtgcagcctcacttaattttcacaccagtgccctgaggtttctagaaaagaggaagggccccagtttaggtggcagtacttttcaatttggaggcaggagatcccagttttcctctgacctctctgagcttccgtgtttttacctctaagacagagagaccaatctctgttcagccaccttagaggattgttttgagggtctataagatgattgagggaaagctctgaaactggtaagagttaaatgtatgtttgtgttatgttgatacgagattcctctgagatgctctttagtccaggcaggtttatggctcacacagcgttcctagggtctcattcttctgcacaggcaggcagctcccgagccagccagtctggacccaaatgccacctgtgctcttggctggtcttgcgcattcaccgaggctcactgtaccttggtgttcccatttctaaaataaggcctgtagtaatccctacctcactgagttgttgagagacttcatgtaaattgtttagcagggagcctggcacagaacaagagcctagcagaaaagtaagctgttgttactttaagcaaatatttagcagcctaaaatggtggctttaccagtttctgtaatggttgaagtgatgacagaattcagccaatcccacccatagtttttcttgctaataaagcagtttgtttctaagtgtgttttgatgcgtataattaatacttcctgtccttgatttttggctgaaaaatgtcttctcttcctcgcaccctctgttattcccgtagtgaatattttcattgtggaagagaaattcccattggctcatggttggcattttatttctcctgtccagagacaatatttccacagcctcaaacgtgaaaactgaaaatctggtgccacttaccaccttgtaaaatcaagtcattgtaattgtgccagtataatcattgatctgaaataaaagcagaaaataaatcaatgggtagctttcttaaatacataagaactaaagatcaaatgggatattggaagactttaacttttcctccataatggtggatcttaattaaacacacagaagaacaatttataaaataaattcataggtttgtttgttcttttttagtgtcaatttttaaaactatctcttttttaattaaaaaaaatagagatgggtctcactatgtcgcccaggctggtctcaaactcctgggctcaagcaatcctcccacctcagcctcccaaagtgctgggattacaggcatgagccactgcacccagcctaaaactgtctcaaaagtagacagcataatgaactcccatgtacttatgatgtggctttaacaattatcagcttatagtcagtcttgttttcttacataataaagttacagtttcaaagccattaggaagaatagcttgctaattataattacttagttttgatagtgtaactttgtcctcagatgggtagaatcgttactctggaacaggggtgtccaatcttggcttccctgggccacattgggagaaaaattgtcttgggccacacgtaaaacatactaacgatagctgatgagctaaaccaacaaaacaaatcccacaaaaatctcatttctttttttttttgagacaagagtctcactctgtcgcccagactggagtgcagttgcacgatctcagctcactgcaagctccgcctcctgggttcacgccattctcctgcctcagcctccctagtagagggtactacaggcgcccgggggtttcaccatgttagccaggatggtctccatctcctgacctcgtgatccgcccacctcggcctcccaaagccctgggattacaggcgtgagccactgcaccgggccaaaaatcttataatgtttttaagaaagtttgcaaatttgttaggccacattcaaagccgtcctgggccatatgcagcccgtgggccatgggttgaacaagcttgctctagaaattggttttcaggccgggcgcggtggctgacgcctgtaatcccagcactttgggaggccgaggcaggtggtcacctgaggtcaggagttcgagaccagcctggccaacatgctgaaaccctgtctctactaaaaatataaaaaattagccaggcgtagtggggagtgcctgtaatcccagcactttgggaggctgaggtgagtagatcacttgagaccaggagttcaagaccagcctggccaacatggcaaaaccccgtctctactgaaaatgaaaaaaaaaaaaaaaaaaaaaacttagccaggcgcagtggctcacatctgtaatcccagctactcaggaggctgaggcaggagaatcacgtgaacctgggagctggaggttgcagtgagcctagatcgcaccactgcacttcagcctgggcgatggagtgagactccaactcaaaaaaaaaaaaaaaaaaatttcaattggcttctattaaagcttttttatgtttgtcattgttttctagtaataacaacaatccacattttttgtgagaaatataccataatatagagaagtataaagaaaatgtaaggccagacacggtggctcacgccgataatcccagcactttgggaggctgaggcaggcttatcacctgaggtcaggagttcgaaaccagcctggccaacatggtgaaaccccatctctactaaaaatacaaaaaaagtagccgagtgtggtggcgggcacctgtaatcccagctactcaggaggctgaggcaggagaattgcttgaacccggaaggtgaaggttgcagtgagcagagatcacgccattgcactccagcctgagtgtcaagaacgagactccgtctcgagaaaaaaaaaaaagaaaagtaaatttaaagcacttaaaatcccaccaccctgagttaactactgttcacagtttggtgaaattcctcctcgtcttctattttttcatagttgggaccatgcctatatactggaatgtaatataaacacttaatatattattgcgagtatattctcaaacttaaatattttttccaaaaatattttgagtggcttttaaaatatatcctattttatgcatgcagcataaactaacttatcctgtgttgatagatattttgtacttgcccttttctgtgctgttgtttaaaattagtgtatagaaataattgtgttgatcacttaggataaattcctagaggtaaatcagatattaaattctttatagacttctggttcatattccagttgccctcaatttatacacctactactagcattgtatgagagtgcccgtttctctgaaatcttacccacactggctgtcagcgtcaaaaagatgtatctgcagatttggcaaggaatttgcatttcagtgattattatcaagcgttcacacactttccatgtttaatggtaattttttttgtttttttttgagacgatgtctcactctgttgcccaggcagagtgcagtgccgcgaccttggctcactgcaacctctgcctcccaggttcaggtgattctcctgcctcaactccttagcagcttggattacaggcatgcgccaccatgcccggctaatttgtgtatttttagtagagacggggtttcaccatattggccaggttagtctcaaactcctgacctcaagtgattgcccgccttggcctcccgaagtggtgggattacaggtgtgagccaccgcacctggcctttaatgataatgtaaattttttgtgaattgtttattcgtggctttgccccattttttccatagtggagggatttttctttttcttgttggtatgtaaagtccttgtgtgtcaagaatgctctgcctcacagtttttctaatataatgacagtggaagctcgtaactggcttccacttgactgacttgctaaattgaccggcactcttcacttccttataaaacatgcctccatagtccctgtgccccatggcacatgggatggggtagccacactaccactgtctgctgctgcatatgttcctgtccacaagagccagtcatgtttgtttccaagcctggttctttcagatatagctgttgttgtaattacttggttgatatgaaaccaatgaaatgcaagtgtgaaaagagaattgtttctaggaaaattaagatgaatgctttagaaagactcagtaaaggaagataaatttaacaaattactacagaattaggtgtaagacaacagtaaatgattggaatgaattacagaaatctagaaagctttgacagtgggaatgcgtagtgaatggctctaagttctccctccatctgtaggaaattgaagctggaagtgagatgatatattcattatagggaggacttacagaagaaaacatcaagctctaatcacatgtccacatttcgttttgtttacttattttttgaaatggagtctcactctgttgcccaggctggagtgcagtggcatgatctcagcttactacaacctctgcctcctgtgttcaagcgattctcgtgcctcagcatcctgagtagttggggctacaggtgtgcaccaccacgcccaactaatttttatatttttagtagagacggggtttcgctatgttggccagcatggtcttaaactcctgacctcaggtgatctgcctgcctcggcctcccaaagtgctgggataacaggcatgagccaccgcacccagccccacatacctacatttcaaaggacagtctttggccctacagtcaagactaccaaaccaatgtacactaaatgggggtggggatcacgttgttgttttttgtttgtttttgtttgtttgtttgtttgtttgtttttttgagagagtctcgctctgtcacccaggctggagtatagtggcgtgatctcggctccctgcaagctccgcctcccgggttcacgccattctcctgcctcagcctcccgagtagctgggactacaggtgcccgccaccacgcccagctaatttttttgtatttttagtagagacggggcttcaccgtgttagtcaggatggtctcgatctcctgacctcggggatcacatattttaataaaattgataatccctaatagtgatggcgctaggtgtacttttagagccaatgtaaatagtcgagaaagggggccaaacatatgactaagcttcttctacactctaggagatgtcagtattgcaagatcctgttccttgaaattatctttgccccccgccattcagaaaaattaccaaatattgtaataatgtaatttaggacttaccctttcagagaaacagttcctgaaggttgacttcagttagtagtacagaagcgagacttgaagctcttcatggtttatgttctgttggtttatcccaaagagatgttttagagcacattcatttctgaaagttctcggtcatgtttccggaagtttgatcaggtgggttctttttctttttttaaaagataattttttttttaaagataattcagaatcagtcccacccctgagatggtattattacccaggaaagaatgcgtgaggatcctctaaatccatagagaaggaaaactaaaacaattttgttaccatttgtttggctcaagcatctggtagatccttcagttcttttcaaattagaattttcctctctttttcttttttttttttttttttgagatggagtcttactccatttcccaggctggagtacagtggcacaatctcggctcactgcagcctcacctcccgggttcaggcaattctccggcctcagtctcctgagtagctgggattacaggtacccgccactaattttcatatttttgcctggctaatttttgtgtttttagtagagatggggttttgccatgttggccaggctggtctcgaactcctgacctcaggtgatctgcccacctcagcctcccaaaatgctgggattacaggggtgagcctccacgccgggcctctctttttaagataataaaaattactaaacatcataatgacatgatttgggattactctttcagagaaacagttcctaaaggtttacttcagttagtgatttttatttttattatttatttattttttgagatggagtctcactctgtctgccaggctagagtgcagagacgagatctcagctcactgcaacctccgcctcccagactcaagcgattctcctacctcagcctcctgagtagctgggactagtagctggaactacaggcgcccgccaccatgcccggctagttttttctattttagtagtgacggggtttcaccgtgttgcccaggttggttttgaactcctgagctcaggctcaggcaatctgcctgcctcagcctcccaatgttctagggttacaggcatgagctactgtgcctggcccagttagtgatttttaaattgtagttccttaaatgagtattttggatttcactccacaatgttcttcaactgtaagatctacttgaggttatttgatttgctggtttgcaagaactcgtctacacgttgtctttatcacaaagaaattctggagcatttgagccctgttcaaaaaaaaaaggcaagaagaaacttcaggaacaaataacctatttgaccctgtaatgaataatattcacacattttctaatttaagagctgttgattgatttccaacttttagaatctgtttcagacaaaacatggaagtttagtaacagttttttctattctgtaataataattgccacaaggttaaaaaaaaaaatttacagaagaacatatttgagggtggtcgtgggggcaagaaagctggaaggacgggtggggtgtgttaatagccttgtcttgtgaagtaagtcctcagagacgctgtcctagttggaagaacaggcagtgaaggtgtgcgtctgttacggagaataggactccagtgatctagctggtacttagagaggtgggggtgatcaccaaaaaaacagctctgcctctgggagcggagaggactgggatagaaagtatttgtctttcattttaagcccttctgtacgatttgacttttcttttttttttttttttttttttttgagatggagtctcactctgtcacccaggctggagtacagtggcacgatcttggctcactgcaacctccgcctcccaggttcaagcaattctcctgcctcagcctcccaagtagctgggactacaggcacctgccaccatgcccggctgatttttgtatttgtagtaaagacaggatttcactgtgttggtcaggttggtcttgaactcctgacctcatgatccacccccggcttttttttttgacacggagtttctcgttacccaggctggagtgcagtggcgcaatctctgctcactgagacctccacctcctgggttcaagcgattctcctgcctcagcctcctgagtagctgggactacaggtgtgcgtcaccatgcccggctaatttttgtatgtctaatggagacagggtttcaccatgttggtcaagttggtctcaaactcctgaactcaggtgatccacccaccttggcctcccaaagtgctgggattacgggcgtgagccaccatgcccggctaaagcaagaaattttcattgcatattttaagcaaaggcaaatgcatatgtggatagactgttttaatttgactaaagtcatattgaatccatgaattttagaagctcaaactattggggaacaataattaccaccttggagtgaaaatacttaatttccacaagatttagtaaaggaagagttttttaaaaaccaccttaatgataatagtatgtacagatgttaagaaatgaaataggaatgtgtaatgttggaaacacaaatatttttgcttctgagaataaaactaattttttctcccaattttctcttcctttttcttttttctgttcccccctttctcttccagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtaaaagggttgtgggcagctagtggtggttgcaggagatagaaatctgggaattgcggtttgacctaccaccctttgctcgttaaaggagcagctttgaaatctggactgcagggatatccaaaacaacaactgcatgtttctaagggagtcgactctccttagaggagttcttgtacaatagccctgggcaaaaacagaacttgccctattttttatactgaaaaggacagctggacaaaatactgaacgcaatttttcccctaagaaaaagcattatttccctaaaatgtcttatattaggaacagagcacttgaataaacataattgatttataaaaactgaggctatacacttacctatctgttcagtacaaacaggaagcttcaaatgtaaacgtgaattctcatacacttataaatgcatatctttatgtggacttgttaaaatgaaattggtatttaggaatttggagatttttagtagttacacaagaatcaatgaaaaagaacgaagctggtttccaaagctgatatgtctgatttggttcctttcttctcaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaaggtaggggacccttggcagggggcgctgatgggcccagggcaggggaaccagaggtcctgctgtcggattgataaattattgcaagaaagctcaaccaagaagatgtttaaagaatctttcaggtgggagtcattccattagccttatgaagaccctttattgaggatccgttctgtgatattacaagttcctgggactggtatgattctcttattgtcttgctagagttttgttgttagcaagttacttaaaataggaggaatatctgttgggttttggacacattttttacaataagattcttcctttaaaaaaaatattttataatgatagaaatcatccccaacgagaaaaattcaaataatagataacctttcttagagcgaaagaccactttactatcccctcccccacagacgcctggcacgtgggcttcttcctgcggatgaacacattcatggagaggtgtgtggacatacgtgcatacacgcatgcagttttcagtgaaaaccgggatcaaacccacagcttaccaaacccaaagtgtggtgttgtaagggaggtggcctatatgattttgtggtcttcatataaataatccttaagagtccataaagagaagggaggagttaacattgactgaggtattgctataatacctgcatctttgcattaaagaaagggaggcataaagagatcttgcccgatgtcaacctagtgagtcactgatggaataggcttggatcccacagtttattctactgcaccaggctgtagttgaggtacagtgttgataggtggtcccgatggtgtatgtttgtttgtttgtttgtttttgagatggagtcttgccctgtcacccaggctggagtacagtggtgcgatcttggctcactgcaacctctgcttcccatgctcaagtgatcctcccaccttagccttccgagtagttgggaccacaggcgcacaccaccatgcccagctgttttttgtttgtttatttgttttgagatggcattttcgctctcattgcccaagctggagtgcaatggtgcgttctcggcttactgcaacctccacctcccgggttcaagcatttctcctgcctcagccttccaggtagctgggattacaggcatgcaccaccacacccagctaattttgtatttttagtagagacagggtttcaccatgttggtcaggctggtctcaaactcctgacctcaggtgatccacctgcctcagcatcccaaagtgctgggattacaggcgtgagccaccacgcctggccgtttttgtatttccagtagagacagggtctcgccatgtcgcccaggctagtctcaaactattgagctcaagcaatctgccctcctcggcctcccaaaatgctgggattacaggcacgagccactgtgcccagcctatattttttaaatagtatgtaatgcatgcatatggttaaaaaaaatcaaaagaacaaaatgaattacagtaaaaagtaatcgtctcctaattcacttttcctgcccagaggcaagcactggtactagttccttctagagttacctggacacagatggatggtatatagatgtttatgtggatgtttatattcaaaaaggcacgcaaatgatacatgcacgcgaatgcatcatgcttctcccgtgtgataatacaccttcgagattgttttatggcaacaggtttagagctgcctcattcttaccagtgactgtgtaccgtggatggactgtcacttatttcagtagtcccctgctggttgacttttaggttgtttccaatctttagttattgtaaataatgctgatatgaggtggctttcaaaccttttcaaaaatctggttctgctgtaagaaaaactcattttacaccaccacccagccctcacaatataacaaaagtttcatgaagtcttacctacccttactgtatgtaacgcactctgatagtttgtatttcattctgttctatttcattttttaaaaattctggttgtaatccactaaatttatttcatagactgactagtggcccatggcctggtttgaaaatgcgttgcacgtgcttctttatgtgggtgtaagtgtgtgcagataagttgccagaactgactgaaggttatgtgcatcaccaggtcaccctctatagaaattgtacaaatttgttctcccacaaacaatccatgaaagggcttgtttccccttaacccacaccaacagtttattattagacatgtaaaaacacctttgtcactctgactggtaaagaatggcatctcattatgttgaatatccatgtggcatattttgggtcatctttatgccatttagtaaattcatctttttcctacatgttagaaatataccaagtgtatatgtgtatctaggcctgttgacctctctgttctcttccattggtctgtctatccgtatgccagtaccaaacttgtaattgctatggcttcataccatgttgtggtatctgataggctagtccccacttacctttttaactttttttttttttggctgtcttcacacattcacttgtaccatatgaacatacatatacttcctttttaatgttcttatttttcttcacatcccaaagtcttctggctgaaatcctattgcccatatctgatgctttccaccactgcagaattctttagtaccaacactgcagtgtgcgtgacggacaaaagagcctgttatccctgtgtgactagacgtgggcctgtcagcttgctttttgggttattgttcttatcccagaggtggagctgcgttcttccattctgagggacgtacttcaggacattgggccctttgaaagagccttcagaccacaagggtacctttctagtgaatcagaacaccagggtgcaccgcccatggggggtgattttctggagcagacagcttgcttcccatgtgtgctatttatccaaggcaagtcatgctgcaccgagatagtcacttgaggtttgcaccgaacaggataatggtttcttttccctagagctgtttaatcagcaaccttggaacagatgcaggcaggcatgatctagtgtgacagctcattagctaggggttgatctaaaatactgttcaaatgggcaaggaaaggcctgcgtatgggatatgtgatttgcagaatttgagattcatccaaagagggagaatctgcctaagatgcctctgtctaaaagctagtatgtgccaggtgggtttgtttcattccaggtagagcacagggagcttatgtgaccaggcagtgtccaccattgggcaacacatcacacttcccttggcgtcttgcctcctccctccccgtctggcacagactctcccatcagccgtttgggcacctggggacttctgagcccacagcgtcatccagcagaccgtctgcggaggttccttcctgtgagggccgcactcgagggggtgtattgggtgggatcgtatcactggaatgagtgaacatcgttagagggagctcccacagctatgcgtgtgaccgtgtggctcgccgctgaaaaatgctgctcacatagcagaacttcgatgcaatcctttttggatttctaaggggaaattttaagaccgataggaggaataggctggggttgaataagtttggttcttatttttaaattagctcatctttagaaaacaactgaatttagaagattcttctgtaaggccccaaaggatttctttctgcctccccgctcttggctcttatttgtgtctctcctcgtgttctgagcttttcagcagcccttgtgggtcatcagccgaggaccatcagctcggggcccagagctggggactgggagttatgagctggactcttgaaggggccaatactaaaccgagccgttgccatttcctaggagccttgacaggctcaaacatctctgggtacccggggatttgactgtcattgttctgatcctttcccaaagttgtaaccctctgttgacatagcggaattgaagattgatttttcttcaccaccttctgcctaaagttcttagttctgaatgtgtccacacctcattgacttctatgagatgcccctgtcacgttcctcgattccttctagtccttaatagagtctttccttttcttgaaagaaatttgcccagttgacatttattttcatcgtctgctgaagtgacagatccaaggccctaagcttcagcagagttgcaggaagggaccatttacacagcctcttagatgtttaaggtttacccgccctgacagtggctcagcaggcgtaggcctcctggtgaaaagccgagcacatggctaacctgctctggtggaggggctttggaccgttcttgtgtactgcgaggcttgccttcctgctggtgtgtgagcagctctcgggggaacttgcttcctggctgagcaacctgtaaacagtatgtctgaggtgggactcccgggcccaaagcagccaaaggttgcacaagtctaagctccgttaggcctgtcagtgtggctgtcaaaagaaacgctgtttcttttctttctttcctttttttttttttttttttttttttttttgagacaggctgtcacccaggctggagtacagtggcacaatctcggctcattgcaacctgcgcctcccgggttcgagcgattctcctgcatcagcctctcaagtagctgagactacaggtgcccaccagcatgcccggctaatttttgtatttttagtagagatggggtttcaccatgttggccaggctggtttcgaacgcctgacctcgtgatctgccctccttggcctcccaaagtgctgggattacaggcgtgagccactgctcctggcaaaatgctgtttctaagcgtttctttgacaagtcccatccttcaggttcatgcatgattagggagaggcagcagccctgactgagcacctgagtgttcatggaaccaggtgcatagcgtaatgacatgccattcatgacactactttcttccctgaacatgccatgaattcctgatgattacacagttccccaacaagtgctgctcaaaacctggaactaatcagaaggaagcaccagccttgactgttagtgggacacggggaagatggctgtgaaccgaatatgcggtgcctttgggggagaatggttttagtatttagtaagtgtcagtatgtaagttttatgttgaaaaatgaactaagtcacaacaaggtctataaatttaattattgaaaaggataaagttgggccattcagtgaaacctcctgtaactgaagtctctaacacagtagccaatgggggaatcaaatgagcccaagaagaaattctgcgtaaactaggacttaggggagcccttagcattccactttggagtgaaggaacccgatccgccagttggggaacgctcctttgaggcccatgtgcttcctgctccgacgggagagagggataatgagacttgctcccaggtgccctgccctggagcccagcaggccacacgggctctttgactctgtactgtgccagtgatttccaagatgggagtggtgctctgattgcaggaatgtcttcttggaatggcttttctcacttctctagagtccttggtagggagcacatcatcagctgagctaacgtgagaacaggggttggagcacttgccaccggcagtgaagtaatcggacagctgacagtcttttccagagggtcggggattgggagctgaccccaggtcccgctccgttttctttgtgtgcagcacttgtgttggtgaccagcagtgaagagagtgcgtggacgtcaccgtgtggttatgaagctaacggcagccctcagcggtgccgacttccgctccccgacacattcctgtgtgcacgttcgtgggtctgagcgtgactcatggtgtgtgtccctagagcagttcactcagaggcttctcttccgtgctctcaagaataaaagcagggccgccacatcctgataagtcttgccattcagcggtcctgtcttgcagagggataagccagtgtccccaggccacagtccttacaccttgagcaagttccccagagtgaagcaatctttctagacaagttttgcgtttctcagaattttattttagtgatattgttaccaaaagattgtaaccccaaaatgtgtgcctttggcttgcctgttttaggaattcacttgtgttatctccccaagcaaaacaaaagaaagagaaaccttcctgttgacgaatcctttttaccttcttgagagttgtcaatgggactaacttgtatatttctttgctaccttcttttaaaaacatttttcttatttactattacagacgtatagtaatggtgcctgacataacccacagccaaaggccttggccttgctgtgtgtatttcaggctttttcttcccttttctttttttttaagtaataagatattacagttatagctgaagcctgatcctgttcccttttctccctctcgtcccaagcatcaccactgtcccaaatctgctccttcttcccacatctttgcagtttgggcaagtcttctgaatgcctagtcatgattttaagtcttctttccaccttcagtgatggttggctaccctgagcaagccagtagattcagttatgtgcctggagtccagatgttttatgtggatttttcatgtggaattgtttttggtaaggaagtattcccttgccacaccaatgaaaagagaagaaaacaggtagctggcacccactgcattgttgctttctgccagcctgctccttctccttcctctccagctctgttgtgtccagatgcaaactctacatgcagggtttgaccctaggtcctcacctgctgtagctctcacacggggaccgaatgctcttgtgtcgtaaatccttccttttgcttcctcgtgttggatgtgtttagtggttttcatcttctggtcactatttcattctaggtttatcccctttaaattataaattaaatgaattcttcgcttttcctaccagcaactacccaccaagttctaccatgtgttaatttaatcaacagttatttattgagtacttattacatgttgatttcctgttctcaacattggaatgaagacagcttagggttctgctttcatgaagcttgcatgctcttaggtggagacagacagtaattaagcagataaacaggaaattatgagctagtagtgggtccattgctgagaagtaagacaggtggtaggatgacccgtgccgtgtgatgactttagattgggtggttggggaagacttcacggaccttaaaactggccttggaacgggaagcgagaactgggcacggaagatgagggagtgggagatttcaggcagagggagcagcagcaggtacagaggccctgaggccttttattgtgtctttttgcttgagcgagtaacttagagcacacgtagagaaagacagcagaagtgatcttctaaacactctgtcctgtgtggagagctccttatgtgagattttgctgtgtagtgaattaaggctcagccaaactggctcacgtgagctctttgagcttgcctgtctctgtgggctgaaggctgttccctgtttccttcagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggtaggctgggactgccgggggtgcccagggtacgtggggcaaggcgtctgctggcattaggcgatgcatctgcctggaagtctacctcctgcctgctgtccgagggcttcattggcgccacggaattgacttttccgtcttatatcattcctgtgtctttgtaggagtggaatcattctcatagtccgagtgtgtttccacatatggtgagagctgacaagcatggaggggttttggtgtaaaaagattagtcatttggagaggttttctcattttatggcaaggttcttttaaagccgtggatttccatgctgttcgtgcggcatggagatcacttcctaccgagagttaaggaggaaaaaaagatctctgagttttgaaagaagatttaaccaaaatgcatttgactcttctgtggatttttgttggctgatttggaaggcaggtgccctgggcatccccagtgggttccaattctgcagctgcccagactcctgcaggcagaggtggaagtgtccccgctagaaaggcatccaggaaactcgcttttgaccaactcagatacagtctggtctttcttgttaactgggactttaccctctccactaatgaatagtgtttgtagtttccaggcaggtttttttcttcttttgaagttctttgaaacccttaggacgccaagggaaggaagttttcattttcagccctttgcattcttcaaaatgtgatccaggcttttccctgtggcgagggtgtcccctagtgttgacttacattatttccacttgttttaaatgaactttgattaccttgacctgtgcacagaaatcaagagaggcagcccctttttagagctgttagaaaagaactgaaacatccacctctgggaaaaatagagtttagtttgtgcatgtgtctcagaagtcaggtgcacacattgagactggtgcagccacatgcctgccttttagggactccttgactgacctgatgccttcacccaaaggcatagtgaaatgggaagtatgatttctgactggtatgaagtggctgctttagagaaaagatcacattctttagtcctagaaaccaaaggtggcttcctgatgctcttattcagcacgtgcctgtctttgaggcaggactggcactggggtgggggcatggtttggggttaccttaaggaccctggatatttctcagataggactttttcttttctttttttgagacggagtcttactctgttgcctaggctggagtgcagtggcatgatctcagctcactgcaacctccgcctccccaaattggcgattctccttcctcagcctcacgagtagctgggattacaggcacccgccaccacacccagctaatttttgtatttttagtagagatggggtttcgctgtgttggcctggctggtctcaaactcctgacctcaagtgatctgcctacctcagcctcccagagtgctgggattataggcatgagccaccacacccggccctggactttttcttaattgaataaatatttatcaagttcttcacttctatttgacggttcctgggctcaacactggattagaaaatgaatgggacagagttcttcatctgagacatagggtggtccaggtagtaggaggaagaaggattgcagcgcagcagtcctcgggacaccaccagggggtgaacgaaggagggacgggggagctgtgcaggtgccccagctccttgggttgcacagggttgaagaccagacccaagaaatggggccttgaccagctcagcaaactgagaatggcctagatttcagcacactttcaccaggatttgtcaaatgaaggaagattccaagcgcaaaataccctagatttcaaccagtaaaagcttgtctgaaaacttccaagatcgggttgtacaacttgacatttgaggaaacgctacttcagtagatggtgctgtggcattgttttctgaacaactgaaggaggagactccagagaagggtacctgctggagacctgtgtggtttgtggggtgggtttcttatatcactgaatctaaagtgggtgcctttcagaatgtgctttactaataatgtgttactctgcttttaattatacttctaataattttccccaagagcctaattaacttacgatctcacagttggtaaagaattacaggatgtcattgcgtggatttctgtgggcagtcacccggagtggtatctaattagggaaaaggcagtatgtccctttctggaagtgtgttaactgaaaatacggtactaagcaactgctaaataaattagccctccttcctcctggccgtgcgcagaaacatccctgcagtatgttgcctatttgtcaaataagattagtgtaaatatggggcatttacctttgctatagaagctgaaattgccaggcacggtggcttacgcctgtaatcccagcactttggaaggccaaggctggtggattgcttgagcccaggagttaaagaccagcctgggcaacatggcaaaactctgcctctacaaaaaataccaaaaatcagccaggcgtggtggtgcgtgtgtattcctagctacttgggaggctgacatgggaggattacctgagcccgggaggttgaggctgcagtgagccgtaatcgtgccactgcactcctacctggggagcagagtgagaccctgtctccaaaaaaaaaaaaaaaaaaaaaagctgaaatccttgagtggattctaaatcatggcagtggctaacattcagcttcatcgtgcccttgctgtgtgcctggcagagacctctgtgcttactacatgtcgcatctcattgaatcctcacaaccttaagatccttccaggttgagcatcccggaccctaaaatcccaaatcctaaatgctcccaaatccaaaactttttgagcaccggcatgcagctcagaggaaatgcatactggaacatttcagattttggattttctaatttggagtgctcatctggtaatatagtgtggatattccccaatctgcaaagatctgagctccgaaacacttgtagccccaagcctttcagttaagggacattcaccctgtatcaacattttaatgctgagaaaatggaagagagagaggttaagtgactgacctggcatcacacagccaggagtggcagcatcaggatttgaactggggcactcagctccaaagctcagctcttcttttttaaaattgaacccagattcttatttctcaaataccataaaatatttacataagccacagttatgccttttaaaaaaaaatcaccttttccccccactctcccatttacttcctttgcagtttgtggctacgatctaccttcctcttcaaaacccttgaaaaatccttgcagccttgcccaagcgtccttccctcagccgatttttgggcctcagtttcctcccatccagcctgtttcctatcgctggcttctccaagggcagagccagagtcctcttttgccagcagggggagcaagggcctacttttaaccgcaactccatagtgaacaagtcaagcaaccttgggtcattcatataccttccaggcagaaggaagacagaagagaacaggaaaaaagcaggacactaggctgacccctgcagatgggaagctggaattacagcaaaggctcattgattgggaactgcacccttgaattttgatagtattgggcaggacatagtcttgctatctgtgttaaagggaggacgtgcttaagaaaaccaacagtcaagtacaagaagctctaattttttttgaggtgggggaacggggtctcactgtgttgcccagctggcctcaaactcctggctcaagcagtcctcccacctcagcctcctgagtagcagggatcacaggcttgtgccactgtgcccagctaagaggctgcaattttttaatgaactgtattcagagtttgtacatcagtgtcttttttttctatgaatcagttctctaagtaatagaatttggaaaagaccgctggtgatctagctgtggcatttagattgactcatgagctaactgacaaatttactacttaaacagtggaaaataatgtgcaggatatatttttttgaataggttgtcccaccctcgagttctgtgtctttcctgagtccgcatatgtgaaccttgttttaaccaggtgacagcatcccataggcacggatctctgtcttctgttgtccacatcatgtgtcttggcagttgttccttattgatatacataaggttgtcttgtttttgtatggatatattttaacatgtaccagtgttcagtgggcctttctcttatttccaatattttgctgttctaaacagcagcacagtgattcccctcatttgcatgtatgtgaaactatcctggtgggtccgcttccagaaactactggatcagggaccatgtgcatttatggccttgaccatactgccagagtaccttccaaggagtttgtgtcatcaatttgcactccccagtggtgttgagagagtgcctgtttcctccacccagcacagtgtgtttcggttttatttctcccaggaaatgggtgttagttggggagctccagaaagaagccgagaagaagggcagtcgtacccttggcagttgtacgcagtggctctctggccgcacaaggagggctcggagcctgctgttgccaggttgcacttgctcttgcaagacaggcattccttgcagtgggttctcctctggccagggagatgagttctgggggctcctctacgaggtggtctctcctgggctttcctctcgagcttcaagtcccacctctggagtgtgaatcgccttcctcagtctaactctatccctccagtggtttttttttttttgctttttgtttttgtttttgtttttttgagacagagtctcactctgtcacccaggctggagtacagtggcgtgatctcggctcaccgcaagctccacctcccgggttcaagcaattctcctgcctcagcctcccgagtagctgggactacaggtgcctgcgaccacgccggctaattttttgtatttttagtagagacagggtttcaccatgttagccaggatggtctcgatctcctgacttcatgatctgccctcctcggcctcccaaagtgctgggattacaggcgtgagccaccacacccagcagtctcttcagtgtatttagctgtgttgatcctgattcatctgatgggtgaaggaatattggaaataaggataatccctgtggtgtttcactgtgcctgtaacctctctcacctagcatttggttaaccagaaacagatgtggaaactgatctatctgtagataatggccctgctcagggagggggtccctgctgagagcgagacattgaaagcctgctacacagaaactgctggaaggccaagccatttcttttttctttttctttttttttttttttttttttcagacagagttttgcccagtagttcaggctggagtgcagtggcatgatctcagttcactgcaacggcctcccaggttcaagcaattcttctgcctcagcctcctgagtagctggcattacagactcctgccaccacgcccggctaatttttgcatttttagtagagacacggtttcaccatgtggccaggctggtctcgaactcctggcctcaagttatctgcctgtctcggccccccagagaggccaagccatttttaaaaacagtgttctttttcatgctgtagaatacctgtgcttgggggtgagcaaaaaggaggaaactgattagaaaatacctatgagggccgggtgcagtggttcatgcctgtaatcccagcactctgggaggccgaggagggcagatcacaaggtcaggagatcaagaccatcctggttgacatggtgaaaccccgtctctactaataatacagaaaattagctgggcgtggtggcaggtgcctgtagtcccagctactcgggaggctgaggcaggagaatggtgtgaacctgggaggcggagcttgcagtgagccgagatcgcgccactgcactctagcctgggcgacagagcaagactccgtctcaaaaaaaaaaagaaaatacctgtgagatgttgagtctgggagaatgtatctgtgagaagacaaacaaccctagtattcattaaagaaccagcctttgttttctacaagctatctgttcggtctactcaggaatagagataaactgccttaatagtaataaaagcaagcaccgctgagtgagtgctggcctcatgccaggaactgctcttggcactctgtgcacaagggagatactgacattctttctgttgtgcagatgagaaaacaggctcagagaaggtgagtaacttaccatggtcacacagcagcagagctaggtcagtataatccacaagtcattctcttaaccgctccgccgtaccgcccagaatgttcatctgcatctgggccagcctcgtcagtccgtgcaaagaggccagttgacccatctccctccctaacaagcacagaagagaccaggctgaaaattcatagctttcactcaaagtgtttccttcacctcattcatctgagttagctttattttactatataaagagaattttcttcagcaaaatgataggaatttttttaatgaaagtaaaggaatgtcacagaattgcgtcagggatatttccagtttaacacaactacaaacgtaggctgttttgttgttgttgttttggttttctattttgagatgaagtctcactctgtcgcccaggctgaagtgcagtggtgcgatcttggctcactgcaacctcctgctcccgaattcaagtgattctcctgcctcagcctcctgagtagctggaactacaggcatgcgctaccacgcctggctaatttttgtatttttagtagagatggggtttcaccatgttgaccaggttgttccggaactcaggtgatccaccttggcctcccaaagtgttgggattacaggcatgagccaccacactcggccagctgttgttaatagttaaaattataaattggacaaggtctgattccagcagggggagacaaattcacattgatttttctataagaaaaggaattatttgtactttcctcccttgtgtgtttgtgtgtatgtaagagagacagatgagaagagggctgcctttgagagccatgttccttttgcatgtacatacctctcaagattattacatgggtcttttagaagagggtcagtagctataaaagcaaatgatatttatgaaatgatttcattgtatgtggtttattatgtagtcttcaaaacagaaaaaccccacttgcttcgaaagcttcctttgagagctactttttaaccatattgttgatcaagcaagattcttaaagccccagtcctatacgtgagcagcattgaatccccggatactggcaggcgttcactgtccagcctttgacattagcatgtcaccctggtaaatcagagcactccatgccaaaaaacagcatggtctgctgagcacattaaggagcagagagggctcagaagaattaaacggccaatggggtggtgacagaggtgagctctttccccctcggacagcttggcatggtgactaccaatgaggtttaattgacattaagagcagctcaccatttttaaagagcgataaattgtttccaagtttcagcctcacggagccctgagctaagagataatgggctgacacatagcctcattggtcacgcttcacttgtaaacaggattggccaaagcaagaaggctgtaagtaaaggttggcccgtcctctggaagcctcccccacagcagggtggggaactggggcagccatagcaagcaggaccatcaggacacggtgtctcccaaacgggccggaacatacaaggttctacgtttaatagaaagttgatcaagccttcctctttggggagcctaatttttaagccattcactctctgttcccctgcagtcttttctctccaaactctgttgaaacttggaggcatctcgcttcctcctttggcctttgtagccaggttttcgcagcaaacagaccatatcatttaacacccacctgcttcttgttctgatagtcagttcagtagacagtagcattttttaagtcaaaaagaggcttagtgatgtaggccaaggtcaaggctctgtgtcatggggctctccccagggctcagtgccacggcacttcagacttgaccccagactgaaaagatgagaggacctgtagaagtcacggcctgtctgtcccgggcccatgctcctatctttttctcccccttggcatttagataccatggacttgttaaaatgtgacatgtactgatgatccttatgaatttgttggtaggggagctgctggtgaggattattttagactgtgagtaattgacctgacagacagtgatgactgcttcattaagagcccacgaccacgtgccagaatagttcagcatcctctgttgctactgtactttgagacatcgttcttctttgtgatgcaatacctctttcttgtcatgagggtctcttcccttaaatcaggtacatttcagattcttcaggtgatgttttttaggctgagtgtgttagcaccccttggaaaccaggttccctggggaaagtgtgtcacgtttgtggtgtttgcccgtatagctctcccacacccccacgagctccaagaggtggggaagcacatgtggcctgcctagcaggcaccagaagcaggttgtcggcacatgcaagccagctttgccctgttgcctgggggagaattgaaaagtttggccccaaaggggaaaattctttctgccatcaagttgctggtcagctgtcatggaacctgtctgcagcaatgtggctgtcacaaaacgcagcccaggacgagtatgcgctgaagctccattttgcattaactagtcaagtacttacccactgaaaagcacttcctgaaataatttcaccttcgtttttttccttctgcaggaggacaccatggaggtggaagagttcttgaaagaagctgcagtcatgaaagagatcaaacaccctaacctggtgcagctccttggtgagtaagcccggggctctgaagagagggtctcgcgccgcacccccagggtgacacaggcgctggggaagacgcacgggcggctcactgcacaaaacctcgttggaatatttgtgctctgccgacgttcagccgcgggtaaaatgaggcctgtatgggatgggtgtgtgcgtgtgtgcacatatgcacatgtatgtatgagagggagaatgtgattattttaagtggatacctaaaagcagtcaaatgcaaatctgaaattagtttctgaaacttgggcattttccagagttttctcactgaagtgattctgtaagtagacacataaccatcagacctaaccattcaggggtaaactgacggtggtgaaggtcatttgaggtggggccaggtctgcgtctgaattctgtggcagcctctccctgcgtaaattcaagttcactggcttgagaagaagaaaagagcctggccatgtccctcccacacgagcacagtctcaggatgcaggtgcttgggaccatgttggaagttgggcccaggactgaggagcagagtcagaatccttcagaaggctttttctttagacagttgtttgttcagttgggagcggagccacgtgttgaagtcctcgttgtcttgttggcaggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacaggtaggggcctggccaggcagcctgcgccatggagtcacagggcgtggagccgggcagccttttacaaaaagccccagcctaggaggtctcagggcgcagcttctaacctcagtgctggcaacacattggaccttggaacaaaggcaaacactaggctcctggcaaagccagctttgggcatgcatccagggctaaattcagccaggcctagactctggaccagtggagcagctaatccccggagtaaggaatatttcatttttgaaccattttgggctaagtggaatattaaatgaagttcataaaaggcgtccttcctaataatcacagtcccctctgaaagagagtactcattgttcctccggggccagcgcccagcagtaatggcctcttcctgggagccgctggcattgcttttacgcaaactcatgggaacaaccagtacatggcttttgttttttagtgtgtgtgtctttttttcctgctttctctttattatcttttcattctccacttaacttgctgtcctctgattcagggatgtttccacaattgtcagaattgtgtatgtggaggtctaaattaagtgtctttgctgttttaaaccctgaagtgttttgaccttcaaatgtgccacaattatcttggtcttcaaattctttgctggtggaaatggcttcccagcaaagcgacggcctgtgcaggacagagcctgatgggctttgctggcgtcttttatacgagatgttctccacaccaccactgtgacctttcctacccaattagctcaagtatgaaacctctacatgagatttcatagcaaggaataagggtagcatgtgtgaaatgctggcggctccttgtaagacagattttctatgctccatgagagcctgatccagatactcctggctttcaggaaagtgaatcctctgatgctgcaaattgagattgcttgacttctggtttgcctggcacggccgggtccgttccttcttcattcaagcgaacttaaaatgctttatcaaaatcctcttaggcacaactcttctgtatttaatttcactcttgttgctgacgatcaggctatctcacgcagccatcacctcaagctgctgccccctctcattccgctcttcccgtttgccttgtgatttcatggttaatgttcatgaaccaaagattagactcctacctccctgggagaatcaggaagtgagaggaagcagattatacaggagagagttttttaggatacatttgggtacacctcaggaagaaagagggaagtaggaaacacagtttgggtaaagaatgccatgggctttgttttattttgtcccatcaagtctgaataggacaagtggagatgcactctgatagaagtttcttgcaatcaaggactgggctcagtgtttgtctccgtgcctggcatagagtagcaataactgcacttactattctcgacacttcaatcaaagcacttagcatattaattaattacactcacagcaaccctaagaggtaggtgccattattatcgtcttcattttacggatgaggtaactgagtcccagggtaattaacttgctccaggtcatgaggccagggagcccgctcccagcaccagcctctccccatgttgggatctcagggtgtgtttgctgagaggctggcactgtgttaattgccgtgggcattaatacaaacttccagggcattggactcaatctttccattgtcagcattgcacctttgctcagcagtggtggatttgtgaagtggaaggttggccaggagctctcatgggtgaacattttcctttcttagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctggggtaagggctgctgctgcactgaagtggtccttcctgactacaggagggtttttttctgcctctttcttgctcttccctttcttttcttcctttctttttgtttttttgagacggagtctcactctgtcacccaggctggagtgcagtggtgcaatcttggctcattgcagcctctccctcccgggttcaagcgattctcctgtctcagcatcttgagtagctgggattataggcacccgccaccacacccagctaatttttggtatttttagtagagatggggtttcaccatgttggccaggctggtctagaactcctgacctcaggtgatctgcctgccttggcctcccaaagtgctgggattacaggtgtgagccactgtgcacagccacccttttttttttttttttaactgtgtatttggcctgaggtccaaatatggtgcacagtgtatctgctgatctgttaagtctcctctataagccccctctccatgtctttatttcccttgctgtttattgaagaaaccatccttagagtttcgtacgttctgggttttgctcattgtctcccatggtgtgatttaatatgttcctctgacttcagtattcctagtaatgggtggctggatctggaacggcctcatcacattcaggtttgactgtttctcttttagtaagagtggatcaggggagatggtgtgcccttccatcagtagatacaaaatgcttcatttatttccttccttcctttcctcccctctccctcccctcctctcttctccttctccttccgacggagtttcgctcttgttgcccaggctggagtgcagtggcacgatcttggctcactgcaacctctgccttctggtttcaagcgattctcctgcctcagcctcctgagtagctgggattataggcgcatgccaccacccccggctaatttttgtatttttagtagagacggggttttaccatgttggccaggatggtctccaactcctgacttcgtgatccacccacctcggcctcccaaagtgctgggattacaggcgtgagccaccacgcctggccccaaaatgcttgatttgctatcgtttttttgtgggggtttttttttgagtaatttttggcagttactgatatacagtgcctagatccaatagtttgctaggtattgccaggtaggaattctctaattctgtcattcctgttaagttactagtggaaacttgtatgaggagaaacagttcctcatctgtgtggttactcagtggtacagttcatgtaggaagagcagggtcaattcttgcttctttgtttttatttaccagtttttaaaataacaaattactttttcttttttttttttggagccagagtcttgctttttctggggtacaaagtacaggctggagttcagtagcatgatctcggctcactgcaacctccgcctaccagattcaagcaattcttctgcctcagcctcccaagtagctgggattacaggtgcctgccaccacgcctggctaaatttttttgtatttttagtaaagatggggtttcaccatgttgcccaggctggccttgaacttctgagctcaggcagtccacctgccttggcctcccaaagtgctgggattacaggcgtgagccaccatgccctgccacaagttggtttcttttttttttttttttttttttttttttgagacagtctcactctgtcgcccaggctggagtgcagtggcgcgatcttgatcttggctcactgcaagctccacctcctgggttcacaccattctcctgcctcagcctcccgagtagctgggactataggcatctaccaccacgcccggctaatttttttgtatttttagtagagacagggtttcaccatgttagccaggatggtctcaatctcctgacctcgtgatccgcccgcctcggcctcccaaagtgctgggattacaggtgtgagccaccgtgcccggcccctaatttttgtatttttagtagagatggggtttcgccatgttggccagggtggtcttgaattcctgacctcaaataatcctcccacttcagcctcccaaagtgctgggattacaggcgtgagccaccgcacccagccttgtcctggtcttctgatgataaagagcctggtaaaatgtcagagcctgggctgctgctggggccatcccttctgaggtctgctgcaaaggtaactgattttaaatgtagtgtagtgaaatgctacacatcttgaacagcctttctctttcggttttctttcagcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgtaagccttcctcagcctgttctcacgagtatatgtgggcattccaggaaattcaactgtgcaggagtgtgtacacaaagttgaaagtttttccatgagctctctccattccagttcttcagatgcagctaatgtagccatttgatacctattgacctttatttacagataaatagtatgtgcgtgacttgtcttttaaagcaaaaatggtattgatagataccaaacctgggtgtattcctaaatacagattcctgggccctgctttcacagacattctgctatagtagctaagctcatgaggtgattttttttttttttttttttgagacggagtttcgctcttgtcgcccaggctggagtacaatggcgcaatgttggctcacttcaacctccacctccctggttcaagcgattctcctgcctcagcctcccaagtagctgggattacaggcatgcaccaccacgcccggctaattttgtatttttagtagagatggggtttctccatgttagtcaggctggtctccaactcccaacctcaggtgatctgcctgcctcggcctcccaaagtgctgcgattacaggcgtgagccaccacgcccagctgcgaggtgatttttatctggtcgttttatactgattacatatgtgttatctgtactatgcacacacaggatgttttcatatatcttataaggtatttatatggccatttcttacactgttttcccacacatgtctttccacgtccatatattcagatctctccctctccctacctcttatttatgtatagctgcccagtactccatttcactcattcatccggtcctcttttgatgtgcatttggttgttttacacatttgtttggtttttgcccttataaacaaagcagaaacaaatattcatgtacacgaatctctgggcactttggctggtatttctaaaagttgaactgttggttccaagaactgtgtgggttttaaattttgatacattttaccaaactgttaaaaaaggttgtgccattgtatgctccagtcagacgcatatgggagtgactgttcctggtccccagtactaggctttgtcagtctgtttagtcctcatgttagtctcattttcatgagaatttctttacttcagactttgataaccgtgaagaaagaacaagatagaaggtgagctgtttggcttagtaattttctacacctactagagcgggactgggaaaaatatatttgtaaatgcagttcttgctgtcactgtctctctggggttttacaatccatattcctgccagcatctaacgtcttttcaaattcttaatgtctataacaggacatgatgacattcatcgttttgacttgttgcagcaaaagatggttagcaggattggaatgttgctttcattctagacttttccttgagaactgctagccccgtattgctagccagatctcatggatgatctgacttgggtttcatctgtccaggttggcagtggaatccctctgaccggccctcctttgctgaaatccaccaagcctttgaaacaatgttccaggaatccagtatctcagacggtaaagtacccatcccggggtacctgcagtggggtgaaagggcagccatgtgggactgcagcctgggtcattcggttcacttcctggtgaaagttcacagaccagcctgtcctgagaccagaaagctgggcagaggtgtggagtattgtgctttcttgtctgctgcagccctgcagagttctaagaaatgctaagggctgtttctccggtatccacgtgccttttctttagttgtatgcagatgagcactgttaccttacaaagaaagagaaccaccacaccaagccaacaccagtactgatggctgctggatttttgtttctgtccctgtatgattcttagaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcggtaagtcccccgcttcccccaaccccactgctcttcccttccctgccagaggctacattcaggccatcataggccaacgggaagctgtgaatggagcccgcacagaagggcagccatggcctttgtcaatggttcagcttcggaaggaggaaggttctcctctccccacctgcctcctatcccctccctctgagagtccccgaggagcataggctccagcagtgagttcagtcctgtaggcagaggtgcttctgaagcccgccaaggagctagcccatctcccacctattacccgcggcatctgtggttgctgtctcagagcagattcaacaatagtaagcaccaggctgcgtgacaagcagctcacggtgaaggcactgatgtgagaagctcgctgcagccaggtggaaccaggacacgcacatggacagaacccgtgtcctggtggtctctgggaagagcatctttctggtgccattgcctaggcgagcaagggagttgggatcagctcaggagggggtctcaagacactagcaaaaataataatggccaacatttatgtagtgtgcctactacaggccaggctctgttctatgttcatggttgacccccagaaccagcagtccaggttctgctgcagaccagctgaacaaaagcattgaggggtgtattagtctgttttcatgctgctaataaaggtatacccaagactgggtaatttataaaggaaagaggtttcaccgactcacagttccacatggctggggaggcctcacaatcatggctgaaggtgaatgaggagcaaggtcacatcttacttggcggcaggcaagagagcttgtgcaggggaagtccgctttataaaaccatcagatctcgtgagacttattcactaccacgagaatgtgggagaaacctccccatgattcattgatctccacctgaccccaccgttgacacgtggggcttattacaattcaaggtgagatttgggtggggactcagccaacccatattggaggtgggggataggccaagcatctgggttcatagaggatcctggggatggcaatggtggactccattctaatggactgcagactgggtacatgtgaagggactggacaaagccctacttcttgaagatttatggtgggatgtttactgacagcagaatctataaacctgagttttctttatcacgtactctcattagtaaaaaaaaaaaaaaaaaacagaacatgcagcccatatatatggtatataaagtacgtatgtccactactgtaggagtagagtgcgtatgttatgtaccaaaacgcacatttaaaaggcttgcatgctaaataagtccaaatttgcaatacttcccgccccgctgggccatctcactcacctacttggaggttatggcgtatagactctccagaatcttccacccttcctcctcttatcggttttcctgatttcaggtggctgagcagtagggaggagaggcctctgggaacgctgtgcttattaagaacccagtgatcagcagtcatgttcatttacgcccgtcagtttcatctgaggggtattttgatagtgatctcttggtgagccgagggtgtcaattaactgctcagtgctgaaactgagtggtgcataattcattgcaatttcactctcctggcctccttccactagattaaagttttgggagggctttggtgttccctgattcagatgtcccgcgtgctgccttgggttctgataaatgcatactggtacctctgttgagttacaggtgccaacagaaaacctcttcttgcaagctgacctctgagggagaattctgttactcactagcaggagcttggcaacaccaaaaagatttccttttttttttgagatggagtctcactctgttgccaggctggagtgcaatggcatgataccagctcactgcaacctccacctcctgggttcaagcgattctcctgcctcatcctcctgagtagctgggactacaggcgtgtgccaccacacccagctaatttttgcattttttagtagagacggggtttcaccatgttggccaggatggtctctatctcttgacctcatgatccgcccacctcagcctcccaaagtgctgggatgacaggcttgagccaccgcgcctggctaccagaaagatttcttatgagctatgattcatatttaaagagtgcccattaaaaaactgttggccgggcgtggtggctcacacctgtaatcccagcactttaggaggccgaggtcagcgaatcacaaggtcaggctcccgtggcacaagctgggggctgcccccaccccatgctccccagaggagcacccaagcgtctgggctcagacaggcctggatctgataaccggctctgatgccaccatccctgaggccaggcaagtcccagccgggctacagcactgttgtgagaatgcagatgaggttgtaaaggtgaagcacgtgtcacgcttctctcaataaagccatcattgctgttaggattgtcattaggaaaggctttgggccgggtgcagtggctcatgcctgtaatcccagcactttgggaggccaaggcgggtggatcacctgaggtcaggagtttgagaccagcctaaccaacatggtgaaaccccgtctctactaaaaatacaaaattagccaggcgtggtagcgggtgcctatagtcctagctacttgggaggctgaggcaagagaaccatttgaacctgggaggcggaggttgcagtgagctgagatcgtgccactgcactccagcgtaggcaacaagcgagactccaactcaaaaaaaaaaaaaaaccacacacacgctttggtctctttccctgaagatgggaacaaaaagctggacggccctggcctcttcatccctgcactggtttacttgaaggcagcagccccccacccacccacatcactctctagtgagtataaaaacgatgacaaggccaagcaaagggtcaaaacctgtggctctcctgccagccagctagccgagaggcctatgaggagctctgggaataaggggctgtgtcccacagtggggaagggacaatgggccattatgcacaggagataagaagggatgacctttgacaatttttttgtttgtttgtttgtttgttttgagatggagtctcactctgtctcctgggctggagtgcagcagtggcactctgcctcccgggttcaagcgattctcctctgtcagcctctagagttgtctggagttgtcagctcttccccttgcgtttcagatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccggcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaatgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacattagaattcttggcagattgcttcggcctgtgatcctgccctgttgttctgagcctgtgcggtggcggggccgggatgtagtgggttctggtttcccgacggggctggggcccggcatcaggtcttacagagcagaacggtggcaggttgcccccagctgcccagggcctctgcggcctgggtgcccacagccatctttcaggcacttgccctgctatgctcggaccctttctgtacccttggcgcttacctgctggcagcctggccccaccggcatcactgggagtgggctgtgccaccatacaagccacatctgactggcggtatcaggctgtcgcagcgacgtggaagtggggtgaggaggtcacgtcccgtgcttcttgctggctgagggcaaatccctccccacgtgcgcctgttctctctccacctttccgccctaagcccccaggtgcccttactcactcatgttccttgagccaggtgtcttaggtctttgtgtggatgaggaggagcggaggtgtgaggtgtgctgggccttcagggcctgggaaggccatcccagactgagccccaccgccctttcccagctcagtccccaccgagctgacagcttggaaaggggcggttgggaagtgagtagcaccctctgataatgatactgatcaggccagcaggtttgtcaggggtgcacatcctgctcagcagagcatatgaagatcacagcttggtgaagctccactcgagagacctggagcgagactgctgtgcggtgggtgacaggtggaactccagccgggccggggctggtggagcccactgcgagcagtgccaggcacaagattctccccctctcatttcacagggacaaaaccaggctggggcccccaatttccttgccggttgaaccatagctggccaccagatccaccaagcctggggcttctcttgtcctttgaatggctgcttctcccaggtctgaacgaagacataggacaactctggtgacagccacagctggtggccgcttcctcgtgtcagatgtggacggtcaccccgccctcccagcgctaagcgctgagactgcgtgtccagagtcggctcctgcacgtgaggccaattggtcacactgggccagtgtggaggaagatgcctctcatctcccaaatggggatctttcatgggtgtcacctgagcttatagagcttagtttagtctagccattccaagacagtggtttcttaaccttttctaggtcacaaaacctcctgagaacctaatgaaaactggatcccagccccagaaaaatgcacctgtgtgcacacacccagcagtttccaggtcatttcgagtcctgccttcgaggtccattgttattatttacaaaacaccagcctaaggtattggacattttcttaaaaggccaggcaaggtggctcatgcctgtaatcccaacactttgggaggctgaggtgggtggattgcttgaggccgggagttcaagaccagcctgggcaacatagaccccatctcacaaaaaatacaaaaacaagctgggtatggtggcacctgtagtccagctactcaggaagctgaggtgggaggattgcttgagcccaggaggtggaggttgcagcaagctatgatcgtgccactggattccagctccagagtgagaccctgtctcaaaaaaaatttttttttcttttttctttttgagacagagtttcgctcttgtctcccaggctggagtgcaatggcacaatctccgctcactgcaacctctgcctcctgggttcaagcgattctcctgcctcagcctcccgagtagctgggactagaggtgcgcaccaccagacccggctaatttttgtatttttagtagggacggagtttcaccatgttggtcaggatggtctcgatctcttgacctcatgatccgcccaccttggactcccaaagtgcttggattacaggcatgagccagcgcgcccggcccaccactttttttttttttttttttttttaagacgagtctcacactgtcgcccagtctgtagtgcagtggtgccatctcggctcactgcaagctccgcctcccaggttcata

SEQ ID NO: 9 - DNA, 79 bp, Artificial Sequence
  Description of Artificial Sequence Synthetic oligonucleotide
actttgataaccgtgaagaaagaacaagatagaaggtgagctgtttggcttagtaattttctacacctactagagcggg

SEQ ID NO: 10 - protein, 10 aa, Artificial Sequence
  Description of Artificial Sequence Synthetic peptide
YFDNREERTR

SEQ ID NO: 11 - protein, 17 aa, Artificial Sequence
  Description of Artificial Sequence Synthetic peptide
ETGFHCVSQDGLDLRTS

SEQ ID NO: 12 - protein, 40 aa, Artificial Sequence
  Description of Artificial Sequence Synthetic polypeptide
LSHTPTSSKRWGSTCGLPSRHQKQVVGTCKPALPCCLGEN

SEQ ID NO: 13 - DNA, 79 bp, Artificial Sequence
  Description of Artificial Sequence Synthetic oligonucleotide
actttgataaccgtgaagaaagaacaagatagaaggacatgatgacattcatcgttttgacttgttgcagcaaaagatg

SEQ ID NO: 14 - DNA, 14 bp, Homo sapiens
gcgagcatgttggc

SEQ ID NO: 15 - DNA, 93 bp, Artificial Sequence
  Description of Artificial Sequence Synthetic oligonucleotide
gcgagcatactttgataaccgtgaagaaagaacaagatagaaggacatgatgacattcatcgttttgacttgttgcagcaaaagatggttggc